(19) United States
(12) Patent Application Publication
(10) Pub. No.: US 2012/0045766 A1
(43) Pub. Date: Feb. 23, 2012

(54) JARID1B FOR TARGET GENE OF CANCER THERAPY AND DIAGNOSIS

(75) Inventors: Yusuke Nakamura, Tokyo (JP); Ryuji Hamamoto, Tokyo (JP); Takuya Tsunoda, Kanagawa (JP)

(73) Assignee: ONCOTHERAPY SCIENCE, INC., Kawasaki-shi, Kanagawa (JP)

(21) Appl. No.: 13/202,742

(22) PCT Filed: Jan. 27, 2010
(86) PCT No.: PCT/JP2010/000441

§ 371 Date: Nov. 4, 2011

Related U.S. Application Data
(60) Provisional application No. 61/208,432, filed on Feb. 23, 2009.

(51) Int. Cl. C12Q 1/68; C12N 15/63; C40B 30/04; A61P 35/02; C07H 21/00; A61K 31/713; A61P 35/00; G01N 33/574; A61K 31/7088

(52) U.S. Cl. 435/6.12; 435/6.14; 435/7.23; 435/320.1; 506/9; 514/44 R; 536/23.1

(57) ABSTRACT

The present invention relates to the roles played by the JARID1B genes in cancers and features a method for treating cancers by administering a composition comprising a double-stranded molecule against the JARID1B genes or a vector encoding them. The present invention also features methods for diagnosing cancers by detecting the expression of JARID1B. To that end, JARID1B serves as a serological biomarker for cancers. Also, disclosed are methods of identifying candidate agents for treating or preventing cancer or inhibiting cancer cell growth, using the expression of JARID1B in the cancer cells or the cell proliferation resulted from expression of JARID1B as an index.

### PRIORITY

The present application claims the benefit of U.S. Provisional Application No. 61/208,432, filed on Feb. 23, 2009, the entire contents of which is incorporated by reference herein.

### TECHNICAL FIELD

1. Technical Field

The present invention relates to the field of biological science, more specifically to the field of cancer research, cancer diagnosis and cancer therapy. In particular, the present invention relates to methods for detecting and diagnosing cancer as well as methods for treating and preventing cancer. Moreover, the present invention relates to methods for screening a candidate compound for treating and/or preventing cancer.

2. Background Art

Cancer is a leading cause of death and millions of people die from cancer every year in the world. The number of cancer cases is increasing globally. Treatment of cancer is basically performed by surgery, radiotherapy and chemotherapy. However, the effectiveness of these treatment is still limited. Moreover, as these treatment sometimes cause adverse effect, many cancer patients experience physical debilitations following treatment.

Bladder cancer is the second most common genitourinary tumor, having an incidence of approximately 357,000 new cases each year worldwide (NPL 1). Approximately one third of bladder cancers are suspected to be invasive or metastatic disease at the time of diagnosis (NPL 1-3). Although radical cystectomy for invasive bladder cancer remains the standard of treatment in many parts of the world, nearly half of such patients develop metastases within two years after cystectomy and subsequently die of the disease. In the last two decades cisplatin-based combination chemotherapy regimens such as CMV (cisplatin, methotrexate, and vinblastine) or M-VAC (methotrexate, vinblastine, doxorubicin, and cisplatin) have been mainly applied to patients with advanced bladder cancers (NPL 3-6). However, the overall prognosis still remains very poor and adverse reactions caused by these combination chemotherapies are significantly severe (NPL 7). Therefore, development of a new molecular target drug(s) against bladder cancer is desired earnestly.

Lung cancer is the leading cause of cancer deaths worldwide, and non small-cell lung cancer (NSCLC) accounts for nearly 80% of those cases (NPL 8). Many genetic alterations associated with development and progression of lung cancer have been reported, but the precise molecular mechanisms remain unclear (NPL 9). Within the last decade several newly developed chemotherapeutic agents such as paclitaxel, docetaxel, gemcitabine, and vinorelbine have begun to offer multiple choices for treatment of patients with advanced lung cancer; however, each of those regimens confers only a modest survival benefit compared with cisplatin-based therapies (NPL 10, 11). Hence, novel therapeutic strategies such as molecular-targeted drugs are eagerly being sought.

Histone methylation plays an important role in regulating chromatin dynamic structure. Precise coordination and organization of open and closed chromatin is crucial for normal cellular processes such as DNA replication, repair, recombination and transcription. Until recently, histone methylation was considered a static modification, but the identification of histone demethylase enzymes has revealed that histone methylation is dynamically regulated (NPL 12, 13). Histone demethylases regulate not only the modification itself but also function indirectly by antagonizing the binding of effector proteins to modified chromatin. This is exemplified by JHDM3A/JMJD2A, which displaces HP1 from chromatin by removing the H3K9 methylation mark that HP1 recognizes, and preventing the spread of H3K9 methylation to the surrounding chromatin by HP1 (NPL 14-16). An extensive family of proteins from yeast to human contain the JmjC domain, which has recently been characterized as a histone demethylase signature motif (NPL 17). Despite the increasing knowledge of the prominent role of histone demethylases in transcriptional regulation, understanding of their physiological function is still limited, particularly in the context of human disease.

The present inventors previously showed that SMYD3, a histone methyltransferase, stimulates cell proliferation through its methyltransferase activity, and plays an important role in human carcinogenesis (PTL 1, NPL 18-22). Other studies also indicates that histone methyltransferases contribute to malignant alterations in human cells (NPL 24-26).

### CITATION LIST

### Patent Literature

[PTL 1] WO2005/071102

### Non Patent Literature

[NPL 1] Parkin D M, et al., Cancer J Clin 2005 55:74-108

[NPL 2] Sternberg C N, et al., Ann Oncol 1995 6:113-26

[NPL 3] Ardavanis A, et al., Br J Cancer 2005 92:645-50

[NPL 4] Lehmann J, et al., World J Urol 2002 20:144-50

[NPL 5] Rosenberg J E, et al., J Urology 2005 174:14-20

[NPL 6] Theodore C, et al., Eur J Cancer 2005 41:1150-7

[NPL 7] Vaughn D J, et al., Semin Oncol 1999 Suppl 2; 117-22

[NPL 8] Greenlee R T et al., CA Cancer J Clin January-February 2001, 51(1): 15-36

[NPL 9] Sozzi G, Eur J Cancer October 2001, 37 Suppl 7: S63-73

[NPL 10] Kelly K et al., J Clin Oncol Jul. 1, 2001, 19(13): 3210-8

[NPL 11] Schiller J H et al., N Engl J Med Jan. 10, 2002, 346(2): 92-8

[NPL 12] Shi Y. et al. Cell 2004; 119: 941-53

[NPL 13] Tsukada Y. et al. Nature 2006; 439: 811-6

[NPL 14] Cloos P A et al. Nature 2006; 442: 307-11

[NPL 15] Fodor B D et al. Genes Dev 2006; 20: 1557-62

[NPL 16] Klose R J et al. Nature 2006; 442: 312-6

[NPL 17] Klose R J et al. Nat Rev Genet 2006; 7: 715-27

[NPL 18] Hamamoto R. et al. Nat Cell Biol 2004; 6: 731-40

[NPL 19] Hamamoto R. et al. Cancer Sci 2006; 97: 113-8

[NPL 20] Kunizaki M. et al. Cancer Res 2007; 67: 10759-65

[NPL 21] Silva F P et al. Oncogene 2008; 27: 2686-92

[NPL 22] Tsuge M. et al. Nat Genet 2005; 37: 1104-7

[NPL 23] Sparmann A. et al. Nat Rev Cancer 2006; 6: 846-56

[NPL 24] Takeshita F. et al. Proceedings of the National Academy of Sciences of the United States of America 2005; 102: 12177-82

[NPL 25]Schneider R. et al. Trends in biochemical sciences 2002; 27: 396-402

### SUMMARY OF INVENTION

The objects of the present invention are providing novel diagnostic and therapeutic strategies for cancers such as acute myelogenous leukemia, bladder cancer, breast cancer, chronic myelogenous leukemia, cervical cancer, lung cancer, prostate cancer and renal cell carcinoma, especially bladder cancer and lung cancer.

The present invention is based on the discovery that the expression level of JARID1B (jumonji, AT rich interactive domain 1B) gene, a H3K4 demetylase belonging to the JARID-family, is up-regulated compared with normal tissues, in many types of cancer, including bladder and lung carcinomas. The present inventors further examined the effect of the small interfering RNAs (siRNAs) targeting JARID1B gene on the growth of cancerous cells and confirmed that such siRNAs have the ability to inhibit growth of cancerous cells. Therefore, the present invention relates to use of JARID1B as a cancerous marker, double-stranded molecules targeting JARID1B gene, methods of diagnosing or treating cancers targeting JARID1B gene and methods of screening a candidate agent or compound useful for treating cancers.

One aspect of the present invention is the method for diagnosing cancer comprising the steps of (a) determining the expression level of JARID1B gene in a subject-derived biological sample and (b) relating an increase in the expression level determined in the step (a) as compared to a normal control level of the gene to the presence of cancer. In one embodiment of the method, the cancer to be diagnosed is acute myelogenous leukemia, bladder cancer, chronic myelogenous leukemia, cervical cancer, lung cancer and renal cell carcinoma, more preferably, bladder cancer and lung cancer. The biological sample can be prepared by harvesting, for example, a biopsy sample, sputum, blood, lymph, pleural effusion or urine from a subject.

In another aspect, the present invention provides use of JARID1B gene product as a cancerous marker. For example, if a person detects an excess amount of an mRNA or a polypeptide derived from JARID1B gene compared to non cancerous samples in a biological sample, such as cells, tissues, blood or other body fluids, the biological sample can be determined to be contaminated with cancerous cells.

Another aspect of the present invention is an isolated double-stranded molecule targeting JARID1B gene, which comprises a sense strand and an antisense strand complementary thereto, wherein the strands hybridize each other to form a double-stranded molecule, wherein the sense strand comprises a nucleic acid sequence corresponding to SEQ ID NO: 21 or 30 as a target sequence. The double-stranded molecule inhibits not only the expression of JARID1B gene but also the cell proliferation when introduced into a cancerous cell which may overexpress JARID1B gene. Therefore, the double-stranded molecule may be useful for treating cancers relating to overexpression of JARID1B gene.

In some embodiments, the double-stranded molecule has a length of between about 19 and about 25 nucleotides. More preferably, the double-stranded molecule has one or two 3′ overhangs consisting of a few nucleotides at either or both the sense strand and/or the antisense strand 3′ terminal. In another embodiment, the double-stranded molecule consists of a single polynucleotide comprising both the sense and antisense strands linked by an intervening single-stranded nucleic acid sequence.

The double-stranded molecule of the present invention may be produced from a vector encoding it by introducing the vector into a cell. Therefore, the present invention also encompasses the vectors encoding the double-stranded molecule as well as isolated molecules.

The isolated double-stranded molecules and the vectors of the present invention are suitable for treating acute myelogenous leukemia, bladder cancer, breast cancer, chronic myelogenous leukemia, cervical cancer, lung cancer, prostate cancer and renal cell carcinoma, more preferably bladder cancer and lung cancer.

Another aspect of the present invention is the method for inhibiting cancer cell proliferation, and also the method for treating or preventing cancer. The method of the present invention comprises the step of administering to a subject an isolated double-stranded molecule targeting JARID1B gene or a vector encoding thereof. The double-stranded molecule comprises a sense strand and an antisense strand complementary thereto, wherein the strands hybridize each other to form a double-stranded molecule, wherein the sense strand comprises a nucleic acid sequence corresponding to the nucleic acid sequence of JARID1B gene or fragment thereof.

In some embodiments, the nucleic acid sequence of JARID1B gene is the sequence of SEQ ID NO: 1. More preferably, the double-stranded molecule has a length of between about 19 and about 25 nucleotides. More preferably, either or both the sense strand and/or the antisense strand have a 3′ overhang consisting of a few nucleotides. More preferably, the nucleic acid sequence of JARID1B gene fragment is the nucleic acid sequence of SEQ ID NOs: 21 or 30. In another embodiment, the double-stranded molecule consists of a single polynucleotide molecule comprising both the sense and antisense strands linked by an intervening single-strand.

Preferably, the present method can be applied to acute myelogenous leukemia, bladder cancer, breast cancer, chronic myelogenous leukemia, cervical cancer, lung cancer, prostate cancer and renal cell carcinoma, more preferably bladder cancer and lung cancer.

Another aspect of the present invention is the composition for inhibiting cancer cell proliferation and also for treating or preventing cancer. The composition comprises an isolated double-stranded molecule targeting JARID1B gene or the vector encoding thereof. The double-stranded molecule comprises a sense strand and an antisense strand complementary thereto, wherein the strands hybridize each other to form a double-stranded molecule, wherein the sense strand comprises a nucleic acid sequence corresponding to nucleic acid sequence of JARID1B gene or fragment thereof.

In some embodiments, the nucleic acid sequence of JARID1B gene is the sequence of SEQ ID NO: 1. More preferably, the double-stranded molecule has a length of between about 19 and about 25 nucleotides. More preferably, either or both the sense strand and/or the antisense strand have a 3′ overhang consisting of a few nucleotides. More preferably, the nucleic acid sequence of JARID1B gene fragment is the nucleic acid sequence of SEQ ID NOs: 21 or 30. In another embodiment, the double-stranded molecule consists of a single polynucleotide comprising both the sense and antisense strands linked by an intervening single-strand.

The composition can be used for treating cancer by being administered to a subject suffering from cancer risk, such as acute myelogenous leukemia, bladder cancer, breast cancer, chronic myelogenous leukemia, cervical cancer, lung cancer, prostate cancer and renal cell carcinoma, preferably, bladder cancer and lung cancer.

Another aspect of the present invention is the method of screening for a compound for inhibiting cancer cell proliferation. The compound identified by the screening method can be a candidate compound for treating or preventing cancer. Such candidate compounds may be suitable to treat acute myelogenous leukemia, bladder cancer, breast cancer, chronic myelogenous leukemia, cervical cancer, lung cancer, prostate cancer and renal cell carcinoma, preferably, bladder cancer and lung cancer.

In one embodiment, the method of screening comprises the steps of (a) contacting a test agent or compound with JARID1B polypeptide, (b) detecting the binding activity between the polypeptide and the test agent or compound and (c) selecting the test agent or compound that binds to the polypeptide as a candidate agent or compound.

In another embodiment, the screening method comprises the steps of (a) contacting a test agent or comopound with a JARID1B polypeptide, (b) detecting the biological activity of the polypeptide and (c) selecting a test agent or compound that suppresses the biological activity of the polypeptide or the fragment as compared to the biological activity detected in the absence of the test agent or compound as a candidate agent or compound. In the present invention, the biological activity is selected from the group of the cell proliferation activity, apoptosis induction and histone H3 demethylation activity.

In another embodiment, the screening method comprises the steps of (a) contacting a test agent or compound with a cell expressing JARID1B gene, (b) determining the expression level of JARID1B gene and (c) selecting the test agent or compound that reduces the expression level of JARID1B gene, as compared to the expression level detected in the absence of the test agent or compound as a candidate agent or compound. The expression level of JARID1B gene can be directly determined by measuring transcription or translation products from JARID1B gene as well as indirectly determined by measuring the expression level of a downstream gene, such as E2F1 and E2F2.

In another embodiment, the screening method comprises the steps of (a) contacting a test agent or compound with a cell into which a vector, comprising the transcriptional regulatory region of JARID1B and a reporter gene that is expressed under the control of the transcriptional regulatory region, has been introduced, (b) measuring the expression or activity of said reporter gene and (c) selecting the test agent or compound that reduces the expression or activity level of said reporter gene as compared to the expression or activity level in the absence of the test agent or compound as a candidate agent or compound.

Preferably, the candidate agent or compound selected by the screening method described above is further screened by being contacted with cancer cells, and then selecting the candidate agent or compound which inhibits cancer cell proliferation. It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention.

### BRIEF DESCRIPTION OF DRAWINGS

FIG. 1 depicts elevated JARID1B expression in bladder cancer in British and Japanese patients. A shows a scatter plot of JARID1B gene expression level, measured using quantitative real-time PCR, in normal bladder tissues and bladder cancer tissues classified by clinical stage. B depicts JARID1B gene expression in normal and tumor bladder tissues in British cases. Expression levels of JARID1B were analyzed by quantitative real-time PCR, and the result is shown by box-whisker plot (median 50% boxed). Relative mRNA expression shows the value normalized by GAPDH and SDH expressions. Mann-Whitney U test was used for statistical analysis. C depicts the expression ratios between bladder normal and tumor tissues in Japanese patients. Signal intensity for each sample was analyzed by cDNA microarray, and the expression ratio is the signal intensity in tumor divided by that in normal (1 is normal).

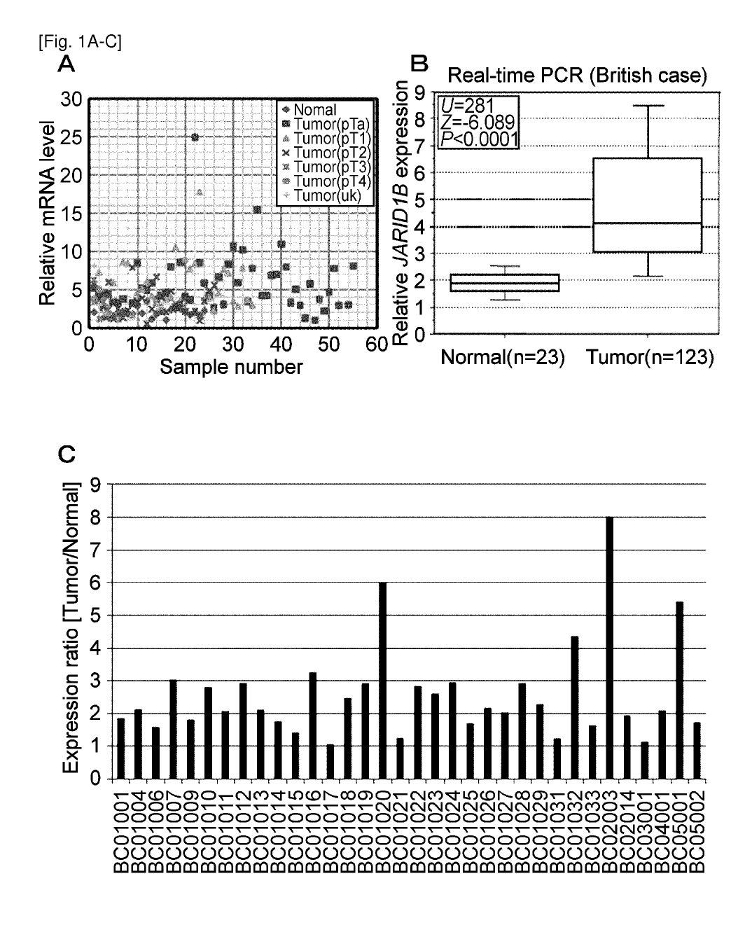

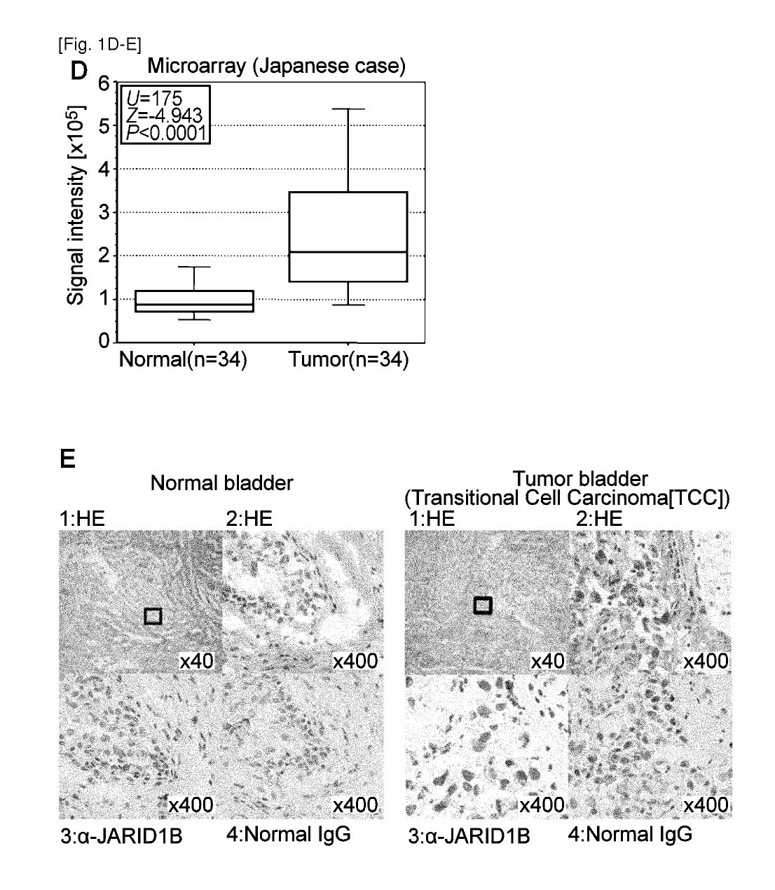

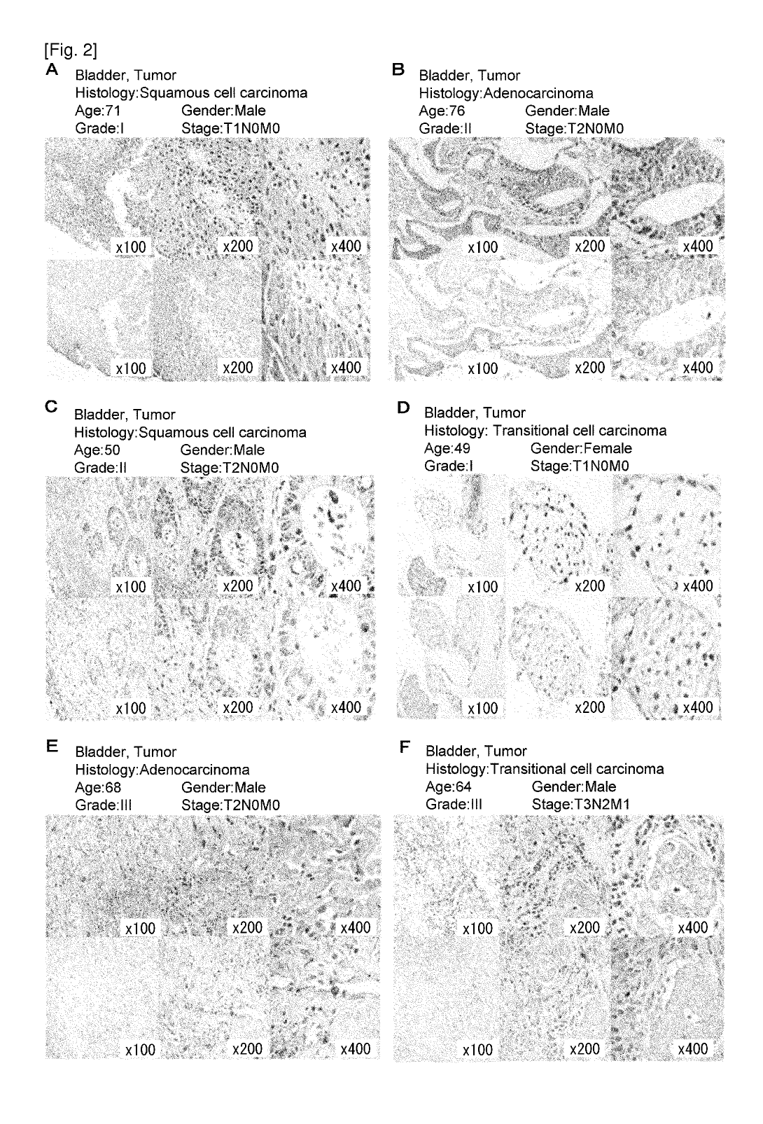

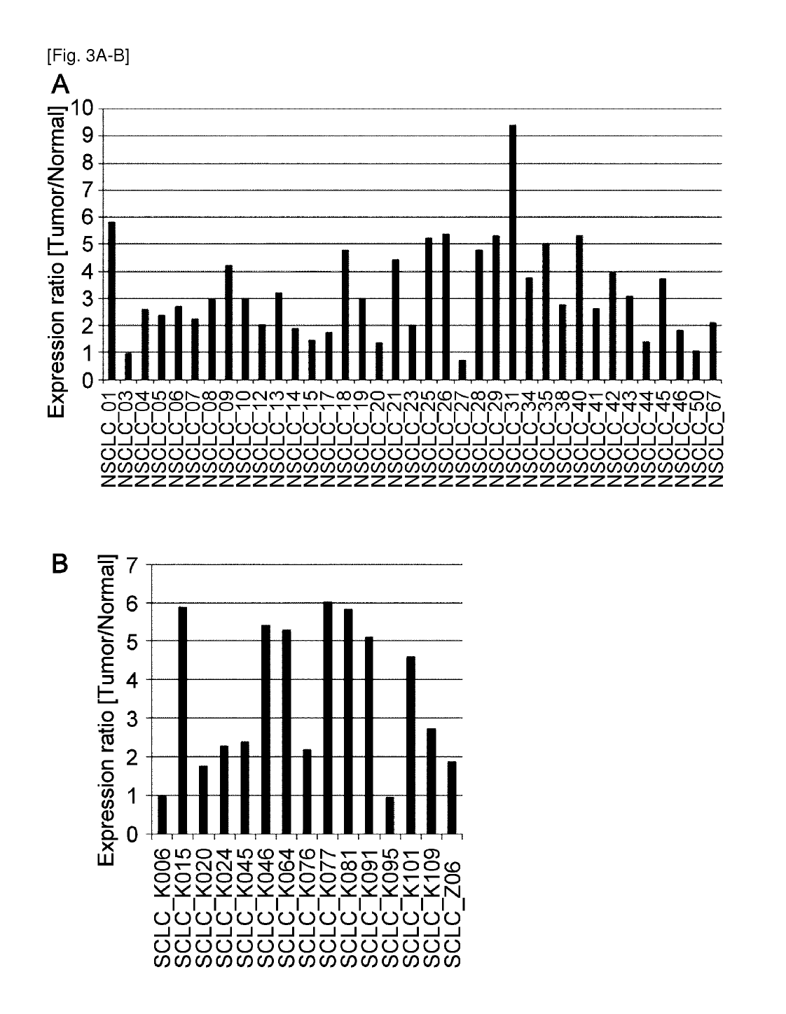

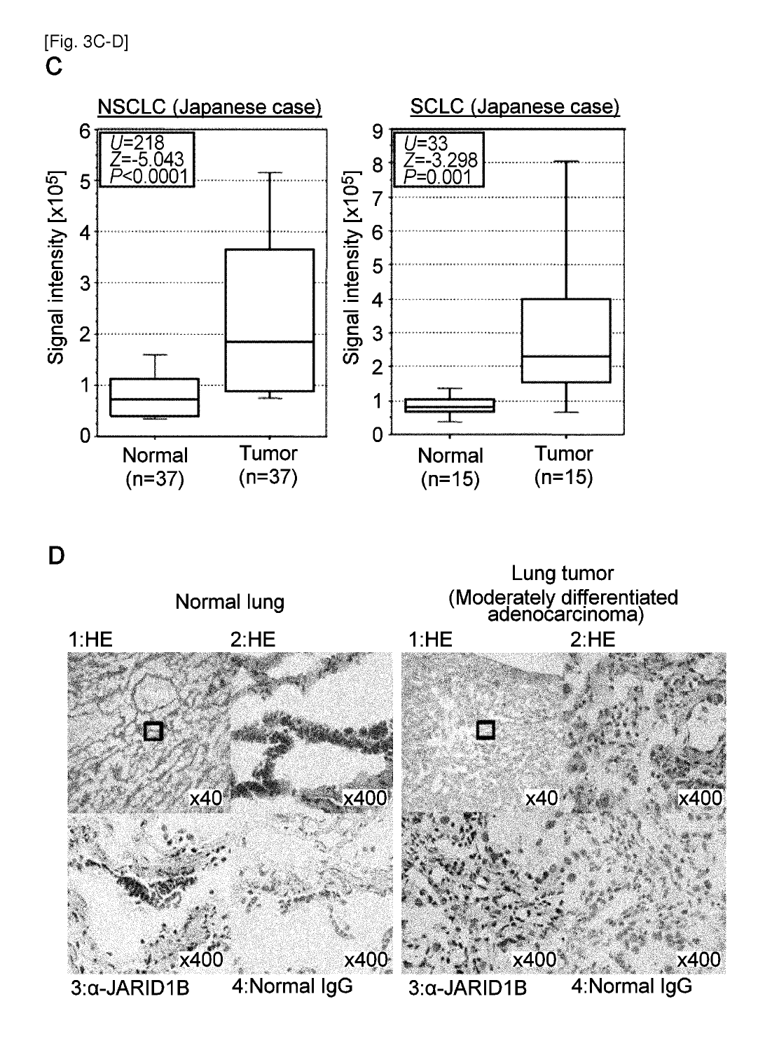

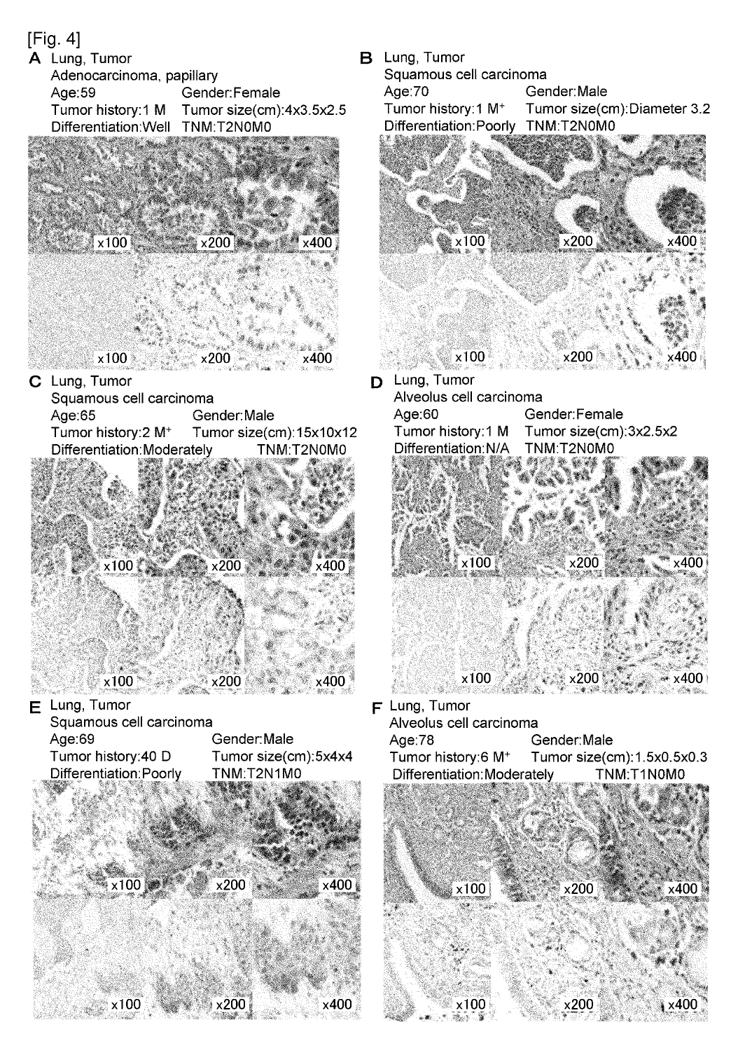

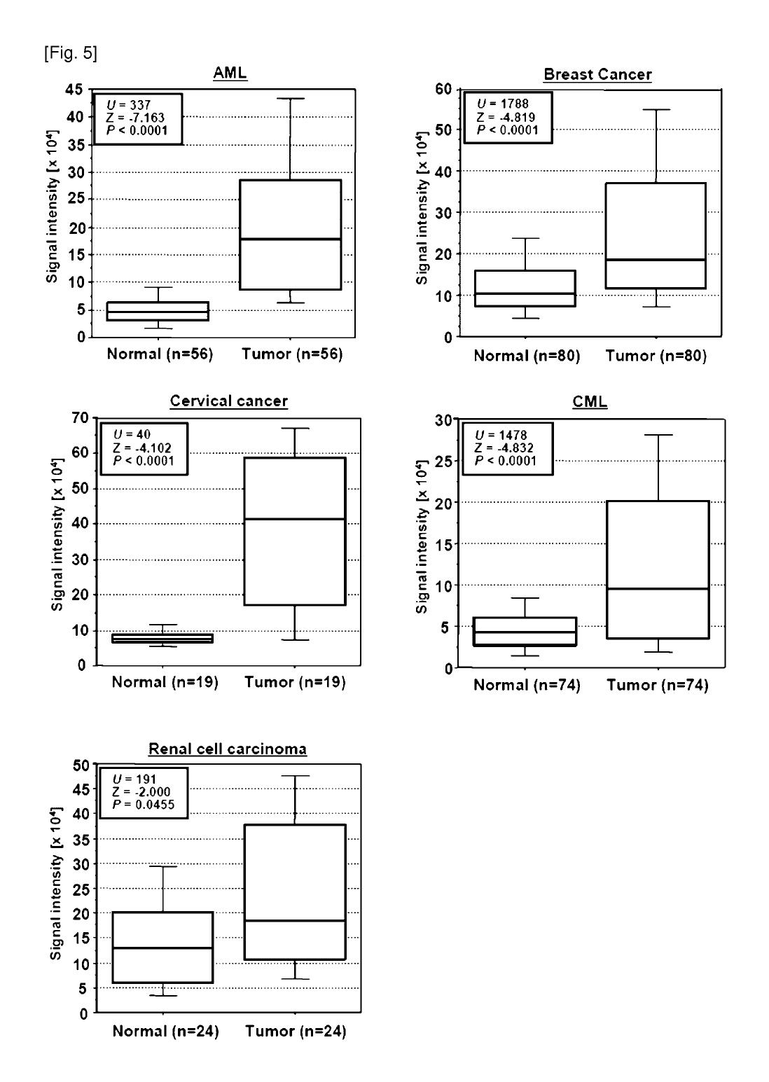

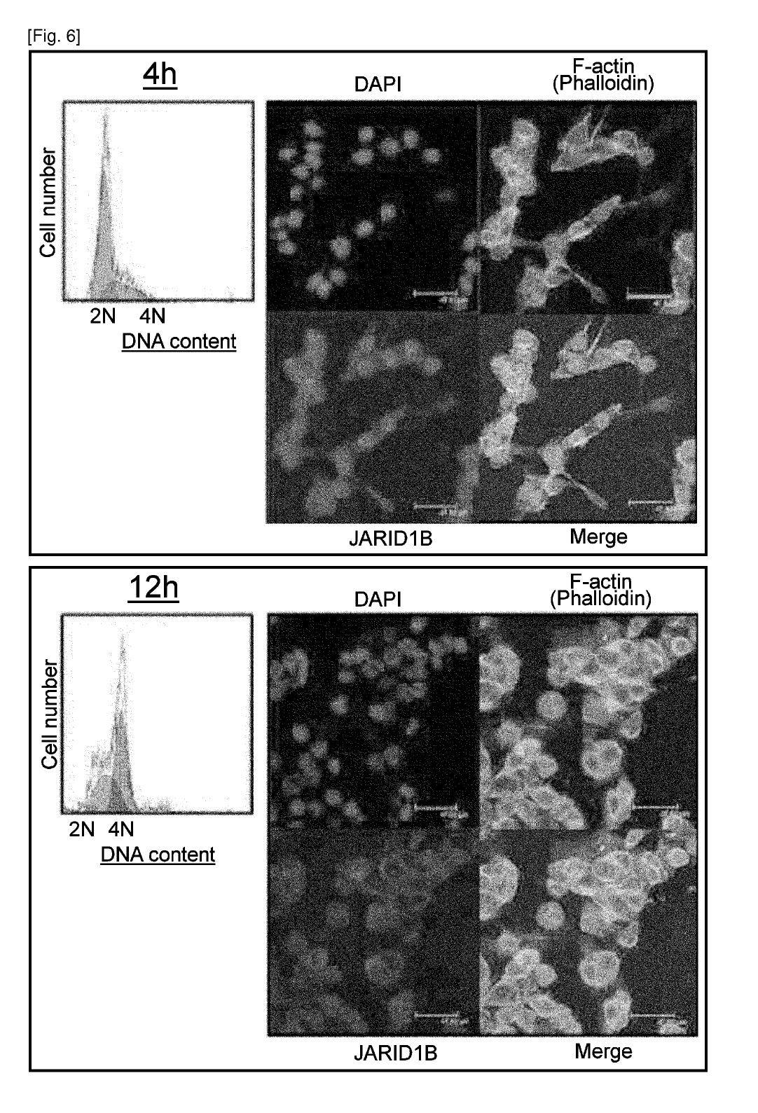

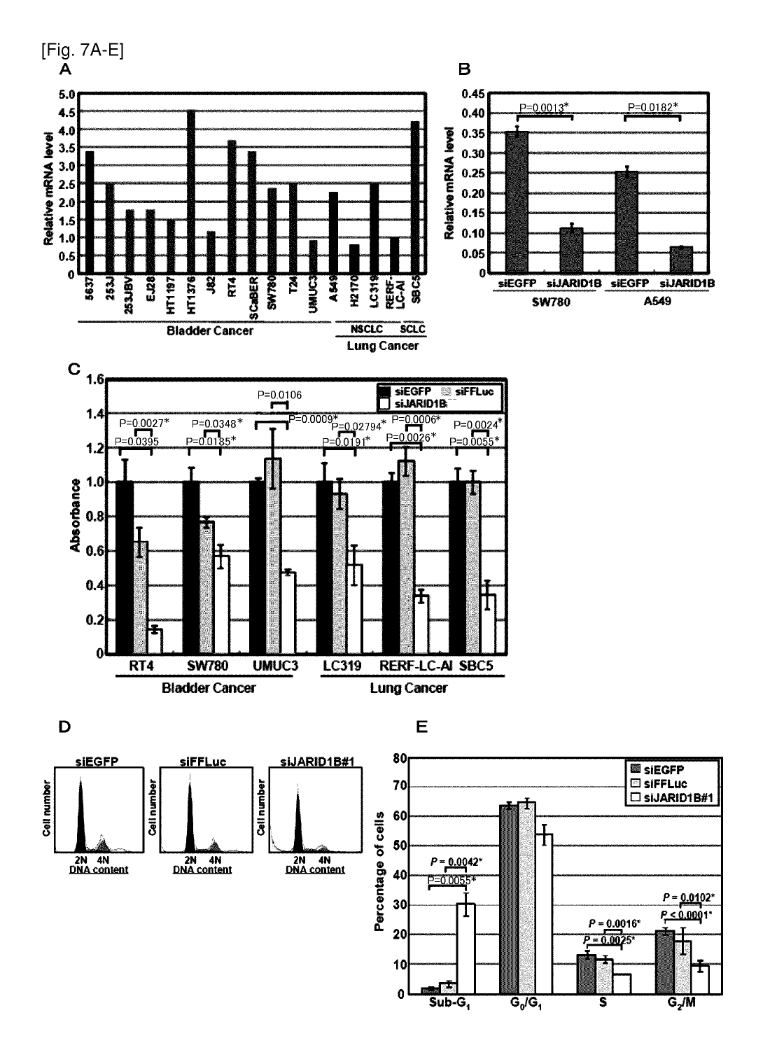

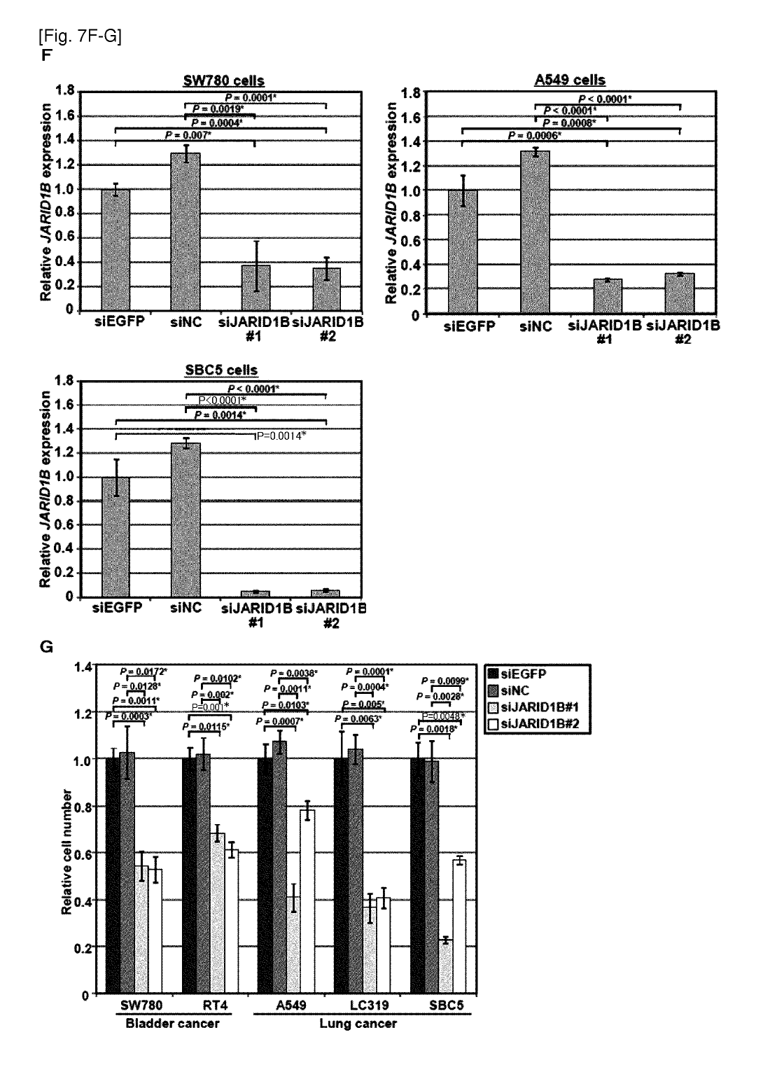

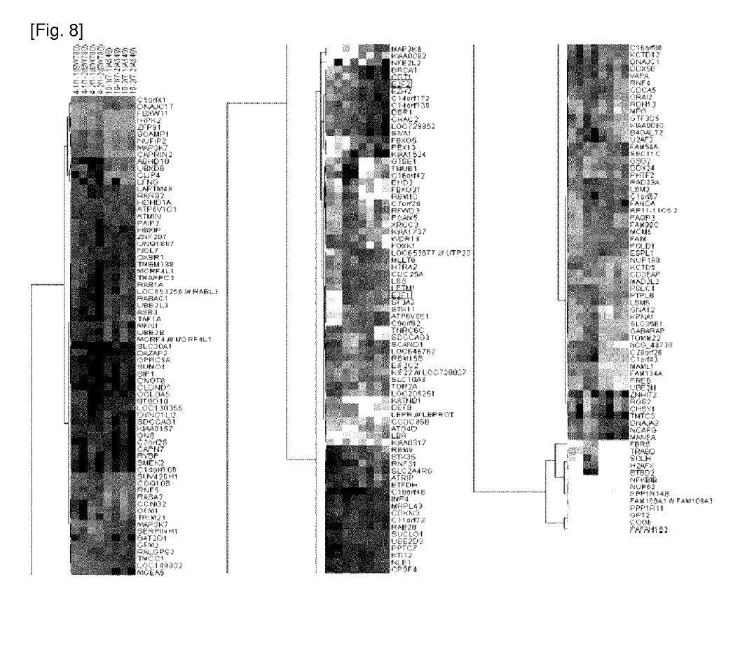

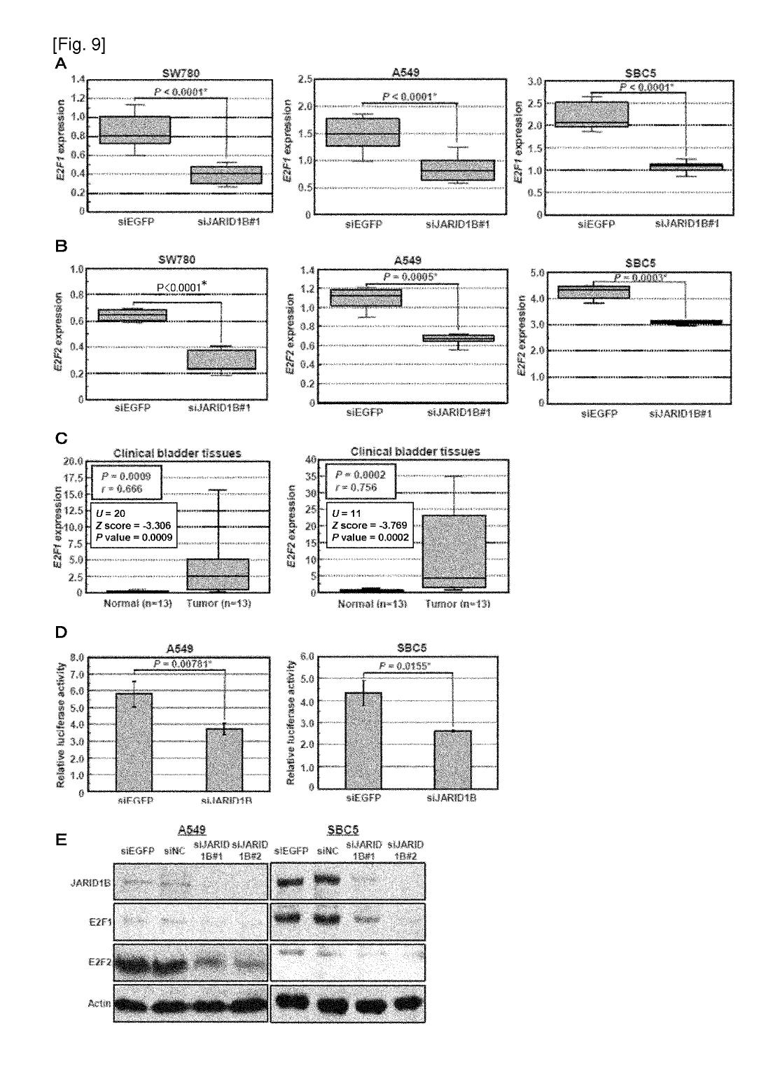

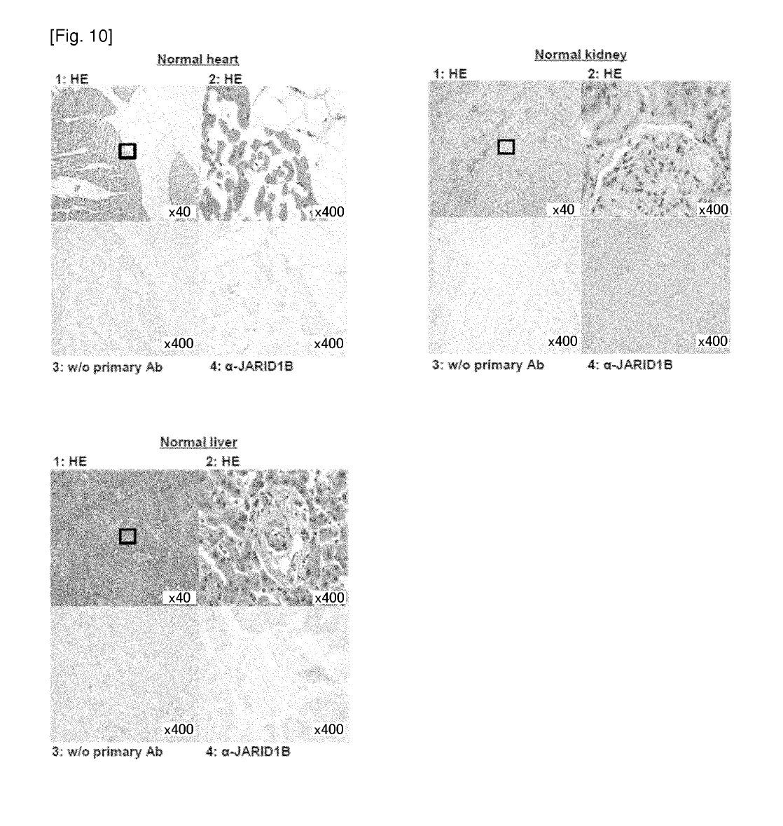

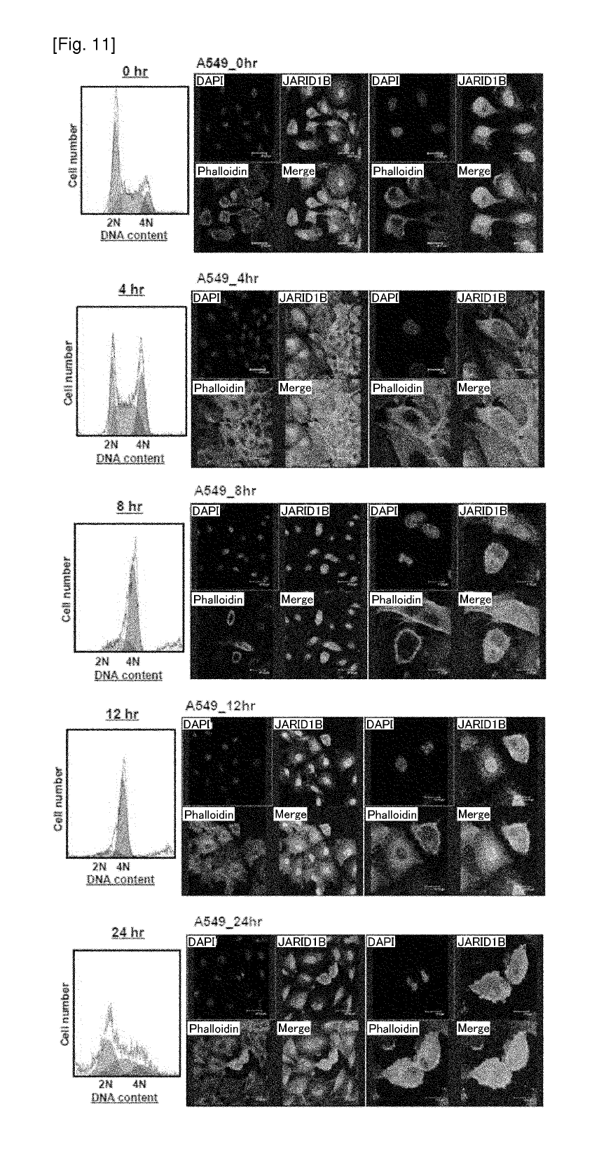

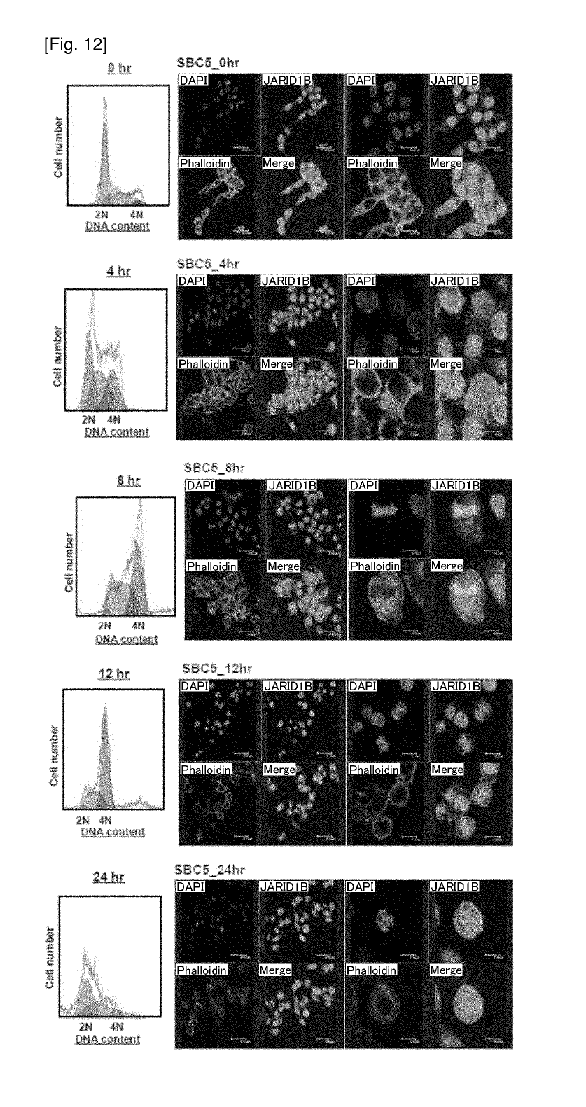

[FIG. 1D-E]D depicts the comparison of JARID1B expression between normal and tumor bladder tissues in Japanese patients. Signal intensity of each sample was analyzed by cDNA microarray, and the result is shown by box-whisker plot (median 50% boxed). Mann-Whitney U test was used for statistical analysis. E depicts immuno-histochemical staining of JARID1B in bladder tissues. Non-immunized mouse IgG was used as a substitute for the primary antibody to eliminate the possibility of false-positive responses from nonspecific binding of IgG or from the secondary antibody. Counterstaining was done with hematoxylin and eosin. Original magnification, ×40 and ×400.

FIG. 2 depicts tissue microarray images of various backgrounds of bladder tumors stained by standard immunohistochemistry for protein expression of JARID1B. Counterstaining was done with hematoxylin and eosin. Original magnification, ×100, ×200 and ×400.

FIG. 3 depicts elevated JARID1B expression in lung cancer. A depicts the expression ratios between normal lung and non-small cell lung cancer (NSCLC) tissues. Signal intensity for each sample was analyzed by cDNA microarray, and the expression ratio is the signal intensity in tumor divided by that in normal (1 is normal). B depicts the expression ratios between normal lung and small cell lung cancer (SCLC) tissues. Signal intensity for each sample was analyzed by cDNA microarray, and the expression ratio is the signal intensity in tumor divided by that in normal (1 is normal).

[FIG. 3C-D]C depicts the comparison of JARID1B expression between normal and tumor lung tissues. Signal intensity of each sample was analyzed by cDNA microarray, and the result is shown by box-whisker plot (median 50% boxed). Mann-Whitney U test was used for the statistical analysis. D depicts immunohistochemical staining pictures using mouse monoclonal anti-JARID1B antibody in lung tissues. Non-immunized mouse IgG was used as a substitute for the primary antibody to verify the possibility of false-positive responses from nonspecific binding of IgG or from the secondary antibody. Counterstaining was done with hematoxylin and eosin. Original magnification, ×40 and ×400.

FIG. 4 depicts tissue microarray images of various various backgrounds of lung tumors stained by standard immunohistochemistry for protein expression of JARID1B. Clinical information for each section is represented above histological pictures. Counterstaining was done with hematoxylin and eosin. Original magnification, ×100, ×200 and ×400.

FIG. 5 depicts elevated JARID1B expression in AML, cervical cancer, CML, breast cancer and renal cell carcinoma in Japanese populations. Expression levels of JARID1B were compared between normal and tumor tissues. Signal intensity of each sample was analyzed by cDNA microarray, and the result is shown by box-whisker plot (median 50% boxed). Mann-Whitney U test was used for statistical analysis.

FIG. 6 depicts SBC5 cells which are subjected to cell cycle arrest by treatment with 7.5 microgram/ml aphidicolin for 24 h and then immunocytochemically stained using anti-JARID1B monoclonal antibody (Alexa594), Phalloidin (F-actin, Alexa488) and 4′,6′-diamidine-2′-phenylindole dihydrochloride (DAPI) at 4 h and 12 h after aphidicolin removal. Insets show FACS analysis demonstrating synchronized release of cell cycle arrest 4 h (upper) and 12 h (lower) after aphidicolin treatment.

FIG. 7 depicts involvement of JARID1B in the growth of bladder and lung cancer cells. A depicts the expression pattern of JARID1B gene in 12 bladder cancer cell lines, in 4 non-small cell lung cancer cell lines and one small cell lung cancer cell line. B depicts quantitative real-time PCR showing suppression of the endogenous expression of JARID1B gene by siRNAs targeting JARID1B (siJARID1B#1) in SW780 and A549 cells. An siRNA targeting EGFP (siEGFP) was used as a control. The mean plus/minus SD of three independent experiments was shown. C depicts effect of JARID1B siRNA knockdown on the viability of bladder cancer cell lines (RT4, SW780 and UMUC3) and lung cancer cell lines (LC319, RERF-LC-AI and SBC5). Cell viability was examined in triplicate. Mean plus/minus SD of three independent experiments. P values were calculated using Student's t-test. D depicts DNA content of SBC5 cells which was analyzed by FACS 72 h after treatment with control siRNA or siRNA targeting JARID1B gene (siJARID1B#1). E depicts numerical analysis of the FACS result in D, classifying cells by cell cycle status. The proportion of cancer cells in sub-G1 phase is significantly high after treatment with siJARID1B#1 as compared to control siRNAs-treated cancer cells. The mean plus/minus SD of three independent experiments was shown. P values were calculated using Student's t-test.

[FIG. 7F-G]F depicts the results of quantitative real-time PCR, showing suppression of endogenous JARID1B expression by two different JARID1B-specific siRNAs (siJARID1B#1 and #2) in SW780, A549 and SBC5 cells. siEGFP and siNC were used as controls. The mRNA expression levels were normalized by GAPDH and SDH expressions, and the values are relative to siEGFP (siEGFP=1). The mean plus/minus SD of three independent experiments was shown. G depicts the effect of JARID1B siRNA knockdown on the viability of bladder cancer cell lines (SW780 and RT4) and lung cancer cell lines (A549, LC319 and SBC5). Relative cell number shows the value normalized to siEGFP-treated cells. The mean plus/minus SD of three independent experiments was shown. P values were calculated using Student's t-test.

FIG. 8 depicts two-dimensional, unsupervised hierarchical cluster analysis of SW780 and A549 mRNA expression profiles after knockdown of JARID1B expression. Differentially expressed genes were selected for this analysis. Red shows Up-regulated genes (left one column); Green shows Down-regulated genes (right two columns).

FIG. 9 depicts confirmation of E2F1 and E2F2 as downstream genes of JARID1B. A and B depicts the expression levels of E2F1 (A) and E2F2 (B) in SW780, A549 and SBC5 cells, which were analyzed by quantitative real-time PCR after treatment with siRNAs targeting JARID1B (siJARID1B#1) or EGFP (control). Relative mRNA expression is an arbitrary value, but for each cell population, expression was normalized to GAPDH and SDH expressions. Statistical analysis was done based on three independent experiments. P values were calculated using Student's t-test. C depicts E2F1 and E2F2 expressions measured by real-time PCR, which were significantly up-regulated in bladder tumor tissues as compared to nonneoplastic bladder tissues, in proportion to that of JARID1B expression in tumor tissues. Each 13 cancer and normal tissues were analyzed and Mann-Whitney U test and Spearman's rank correlation coefficient were used for the statistical analysis. D depicts that effect of siRNA knockdown of JARID1B gene expression on the transcriptional activity of E2F. Cignal™ E2F Reporter Assay Kit was used for this assay. The mean plus/minus SD of three independent experiments was shown. P values were calculated using Student's t-test. E depicts JARID1B, E2F1 and E2F2 expressions at the protein level. Lysates from A549 and SBC5 cells after siJARID1B#1 or siJARID1B#2 treatments were immunoblotted with anti-JARID1B, E2F1, E2F2 and actin antibodies. Actin was served as an internal control.

FIG. 10 depicts images of normal heart, kidney and liver stained by standard immunohistochemistry for protein expression of JARID1B. The control staining was performed without primary antibody to eliminate the possibility of false-positive responses from the secondary antibody, and counterstaining was done with hematoxylin and eosin. Original magnification is ×40 and ×400.

FIG. 11 depicts subcellular localization of JARID1B in A549 cells. A549 cells. These cells were subjected to cell cycle arrest by treatment with 7.5 microgram/ml aphidicolin for 24 hours, and then immunocytochemically stained using anti-JARID1B monoclonal antibody (Alexa488 [green]), Phalloidin (F-actin, Alexa594 [red]) and 4′,6′-diamidine-2′-phenylindole dihydrochloride (DAPI; blue) at 0, 4, 8, 12 and 24 hours after aphidicolin removal. Insets show FACS analysis demonstrating synchronized release of cell cycle arrest, and arrows indicate cytoplasmic localization of JARID1B. A549 cells were fixed with PBS (−) containing 4% paraformaldehyde for 20 min, and rendered permeable with PBS (−) containing 0.1% Triton X-100 at room temperature for 2 min. Subsequently, the cells were covered with PBS (−) containing 3% bovine serum albumin for 1 hour at room temperature to block nonspecific hybridization, and then were incubated with mouse anti-JARID1B antibody (1G10, Abnova), diluted at 1:100 ratio dilution. After washing with PBS (−), cells were stained by an Alexa594-conjugated anti-mouse secondary antibody (Molecular Probe) at 1:1000 dilution. Nuclei were counter-stained with 4′,6′-diamidine-2′-phenylindole dihydrochloride (DAPI). Fluorescent images were obtained under a TCS SP2 AOBS microscope (Leica).

FIG. 12 depicts subcellular localization of JARID1B in SBC5 cells. SBC5 cells were subjected to cell cycle arrest by treatment with 7.5 microgram/ml aphidicolin for 24 hours, and then immunocytochemically stained using anti-JARID1B monoclonal antibody (Alexa488 [green]), Phalloidin (F-actin, Alexa594 [red]) and 4′,6′-diamidine-2′-phenylindole dihydrochloride (DAPI; blue) at 0, 4, 8, 12 and 24 hours after aphidicolin removal. Insets show FACS analysis demonstrating synchronized release of cell cycle arrest, and arrows indicate cytoplasmic localization of JARID1B.

### DESCRIPTION OF EMBODIMENTS

Although methods and materials similar or equivalent to those described herein can be used in the practice or testing of embodiments of the present invention, exemplary methods and materials are now described. However, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the present invention which will be limited only by the appended claims.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the present invention, the following definitions apply:

Definitions:

The words “a”, “an”, and “the” as used herein mean “at least one” unless otherwise specifically indicated.

The term “gene”, “polynucleotide”, “oligonucleotide” “nucleotide”, “nucleic acid”, and “nucleic acid molecule” are used interchangeably herein to refer to a polymer of nucleic acid residues and, unless otherwise specifically indicated are referred to by their commonly accepted single-letter codes. The terms apply to nucleic acid (nucleotide) polymers in which one or more nucleic acids are linked by ester bonding. The nucleic acid polymers may be composed of DNA, RNA or a combination thereof and encompass both naturally-occurring and non-naturally occurring nucleic acid polymers.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

Unless otherwise defined, the terms “cancer” refers to cancers over-expressing the JARID1B gene. Examples of cancers over-expressing JARID1B gene include, but are not limited to, acute myelogenous leukemia, bladder cancer, chronic myelogenous leukemia, cervical cancer, lung cancer and renal cell carcinoma, more preferably, bladder cancer and lung cancer.

JARID1B Gene:

JARID1B, also named Plu1, is encoded by one of the paralogous JARID-family genes in humans (Kortschak R D et al. Trends Biochem Sci 2000; 25: 294-9, Wilsker D. et al. Genomics 2005; 86: 242-51). In addition to a JmjC domain, it contains other domains common to transcriptional regulators, including a JmjN domain, Bright/Arid domain, C5H2C zinc finger, and several PHD domains. All four members of the JARID family possess H3K4 demethylase activity (Christensen J. et al. Cell 2007; 128: 1063-76, Iwase S. et al. Cell 2007; 128: 1077-88, Klose R J et al. Cell 2007; 128: 889-900, Lee M G et al. Cell 2007; 128: 877-87, Yamane K. et al. Mol Cell 2007; 25: 801-12). Although functional redundancy may exist, each member seems to participate in different biological processes through recruitment to different chromosomal regions and differing enzymatic activities (Klose R J et al. Nat Rev Genet 2006; 7: 715-27). The exemplary nucleic acid and polypeptide sequences of JARID1B gene are shown in SEQ ID NO: 1 and 2 respectively, but not limited to those.

Furthermore, the sequence data are also available via accession number of NM_{—}006618 and NP_{—}006609 respectively, for example.

According to an aspect of the present invention, functional equivalents are also considered to be “JARID1B polypeptide”. Herein, a “functional equivalent” of JARID1B protein is a polypeptide that has a biological activity equivalent to JARID1B protein. Namely, any polypeptide that retains the biological ability of JARID1B protein may be used as such a functional equivalent in the present invention. For example, functional equivalents include polypeptides that have cell proliferation activity, anti-apoptosis activity, demethylase activity or promoting activity for the expression of E2F1 and E2F2 similar to JARID1B protein.

Generally, it is known that modifications of one or more amino acid in a protein do not influence the function of the protein. In fact, mutated or modified proteins, having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that individual additions, deletions, insertions, or substitutions to an amino acid sequence which alter a single amino acid or a small percentage of amino acids or those considered to be a “conservative modifications”, wherein the alteration of a protein results in a protein with similar functions, are acceptable in the context of the instant invention.

So long as the activity of JARID1B protein is maintained, the number of amino acid mutations is not particularly limited. However, it is generally preferred to alter 20% or less of amino acid sequence, more preferably 10% or less of the amino acid sequence, more preferably 5% or less of the amino acid sequence. Accordingly, in a preferred embodiment, the number of amino acids to be mutated in such a mutant is generally 30 amino acids or less, preferably 20 amino acids or less, more preferably 10 amino acids or less, more preferably 6 amino acids or less, and even more preferably 3 amino acids or less.

The phrase “conservative modifications” refers to the modification that an amino acid residue to be mutated is preferably mutated into a different amino acid in which the properties of the amino acid side-chain are conserved. Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Conservative substitution tables providing functionally similar amino acids are well known in the art. For example, the following eight groups each contain amino acids that are conservative substitutions for one another:

1) Alanine (A), Glycine (G);

2) Aspartic acid (D), Glutamic acid (E);

3) Aspargine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cystein (C), Methionine (M) (see, e.g., Creighton, Proteins 1984).

Such conservatively modified polypeptides are included in the present protein. However, the present invention is not restricted thereto and the protein includes non-conservative modifications, so long as at least biological activity of JARID1B protein is retained. Furthermore, the modified proteins do not exclude polymorphic variants, interspecies homologues, and those encoded by alleles of these proteins.

A polypeptide of the present invention may have variations in amino acid sequence, molecular weight, isoelectric point, the presence or absence of sugar chains, or form, depending on the cell or host used to produce it or the purification method utilized. Nevertheless, so long as it has a function equivalent to JARID1B protein, it is within the scope of the present invention.

In other embodiments, the polypeptide of the present invention can be encoded by a polynucleotide that hybridizes under stringent conditions to the natural occurring nucleotide sequence of JARID1B gene. The phrase “stringent conditions” refers to conditions under which a nucleic acid molecule will hybridize to its target sequence, typically in a complex mixture of nucleic acids, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10 degrees C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times of background, preferably 10 times of background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 50° C.

In the context of the present invention, a condition of hybridization for isolating a polynucleotide encoding a polypeptide functionally equivalent to JARID1B protein can be routinely selected by a person skilled in the art. For example, hybridization may be performed by conducting pre-hybridization at 68 degrees C. for 30 min or longer using “Rapid-hyb buffer” (Amersham LIFE SCIENCE), adding a labeled probe, and warming at 68 degrees C. for 1 hour or longer. The following washing step can be conducted, for example, in a low stringent condition. An exemplary low stringent condition may include 42° C., 2×SSC, 0.1% SDS, preferably 50° C., 2×SSC, 0.1% SDS. High stringency conditions are often preferably used. An exemplary high stringency condition may include washing 3 times in 2×SSC, 0.01% SDS at room temperature for 20 min, then washing 3 times in 1×SSC, 0.1% SDS at 37 degrees C. for 20 min, and washing twice in 1×SSC, 0.1% SDS at 50 degrees C. for 20 min. However, several factors, such as temperature and salt concentration, can influence the stringency of hybridization and one skilled in the art can suitably select the factors to achieve the requisite stringency.

Moreover, the gene of the present invention encompasses polynucleotides that encode such functional equivalents of the protein. In addition to hybridization, a gene amplification method, for example, the polymerase chain reaction (PCR) method, can be utilized to isolate a polynucleotide encoding a polypeptide functionally equivalent to JARID1B protein, using a primer synthesized based on the sequence above information.

Polynucleotides and polypeptides that are functionally equivalent to the human gene and protein, respectively, normally have a high homology (also referred to as “sequence identity”) to the originating nucleotide or amino acid sequence of. “High homology” (or high sequence identity) typically refers to a homology of 40% or higher, preferably 60% or higher, more preferably 80% or higher, even more preferably 90%, 93%, 95%, 97%, or higher. The homology of a particular polynucleotide or polypeptide can be determined by following the algorithm in “Wilbur and Lipman, Proc Natl Acad Sci USA 80: 726-30 (1983)”.

Use of JARID1B as a Cancerous Marker:

The expression of JARID1B gene was found to be specifically elevated in various types of cancer tissues as shown by Table 3. Therefore, JARID1B is a useful as a cancerous marker in the various types of cancers. Recently, JARID1B gene was reported to be overexpressed in breast cancer and prostate cancer (Yamane K. et al. Mol Cell 2007; 25: 801-12, Xiang Y. et al. Proc Natl Acad Sci USA 2007; 104: 19226-31). However, it had been unclear whether JARID1B will be useful as a cancerous marker of other cancers because the gene expression profile in one type of cancer generally differ from one in other types of cancers. Only the present invention clarified the overexpression of JARID1B gene in acute myelogenous leukemia, bladder cancer, chronic myelogenous leukemia, cervical cancer, lung cancer and renal cell carcinoma for the first time. Thus, the present invention provides use of JARID1B as a cancerous marker in those cancers.

According to the present invention, a biological sample can be determined to contain cancerous cells, consequently contains products derived from cancerous cells, by detecting the overexpression of JARID1B gene. Namely, the present invention provides the method for determining whether a biological sample is contaminated with cancerous cells or cancerous products, which comprises the step of quantifying the expression level of JARID1B gene in the biological sample and comparing the expression level with the expression level in a non-cancerous sample, which is known to be free from cancer.

As used herein, the term “biological sample” refers to a whole organism or a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen). “Biological sample” further refers to a homogenate, lysate, extract, cell culture or tissue culture prepared from a whole organism or a subset of its cells, tissues or component parts, or a fraction or portion thereof. Lastly, “biological sample” refers to a medium, such as a nutrient broth or gel in which an organism has been propagated, which contains cellular components, such as proteins or polynucleotides.

Exemplary biological samples include, but not limited to, biopsy specimen, bodily tissues and fluids, such as blood, sputum, pleural effusion or urine. Preferably, the biological sample contains a cell population comprising an epithelial cell, more preferably a epithelial cell derived from tissue suspected to be cancerous. Further, if necessary, the cell may be purified from obtained bodily tissues and fluids, and then used as the biological sample. However, any biological material can be used as the biological sample for the determination so long as it includes the objective transcription or translation product of JARID1B.

For example, according to the present invention, cancers including acute myelogenous leukemia, bladder cancer, chronic myelogenous leukemia, cervical cancer, lung cancer and renal cell carcinoma can be diagnosed. Alternatively, the present invention provides a method for detecting cancer cells of these cancers in biological samples. In order for diagnosing these cancers or detecting cancer cells, biological sample derived from following organs collected from a subject to be diagnosed or detected can be used as biological samples:

lymphocyte or blood sample including lymphocyte:

for acute myelogenous leukemia, chronic myelogenous leukemia,

bladder: for bladder cancer,

cervical: for cervical cancer,

lung: for lung cancer and

kidbney: for renal cell carcinoma

The expression level of JARID1B gene can be quantifying by detecting mRNA, protein or activity of JARID1B using methods known in the art. The details of the methods are described below in “Methods for diagnosing cancer”.

The present method may provide an useful information to utilization of biological materials, cancer diagnosis, cancer therapy and so on.

Methods for Diagnosing Cancer:

The present invention also provides methods for detecting cancer cells or diagnosing cancer using the expression level of JARID1B gene. The present method may be suitable for diagnosis of acute myelogenous leukemia, bladder cancer, chronic myelogenous leukemia, cervical cancer, lung cancer and renal cell carcinoma.

According to the present invention, an intermediate result for examining the condition of a subject may be provided. Such intermediate result may be combined with additional information to assist a doctor, nurse, or other practitioner to diagnose that a subject suffers from the disease. Alternatively, the present invention may be used to detect cancerous cells in a subject-derived biological sample, and provide a doctor with useful information to diagnose that the subject suffers from the disease.

Alternatively, the present invention provides a method for detecting or identifying cancer cells in a subject-derived biological sample, said method comprising the step of determining the expression level of the JARID1B gene in a subject-derived biological sample, wherein an increase in said expression level as compared to a normal control level of said gene indicates the presence or suspicion of cancer cells in the tissue.

Such result may be combined with additional information to assist a doctor, nurse, or other healthcare practitioner in diagnosing a subject as afflicted with the disease. In other words, the present invention may provide a doctor with useful information to diagnose a subject as afflicted with the disease. For example, according to the present invention, when there is doubt regarding the presence of cancer cells in the tissue obtained from a subject, clinical decisions can be reached by considering the expression level of the JARID1B gene, plus a different aspect of the disease including tissue pathology, levels of known tumor marker(s) in blood, and clinical course of the subject, etc. For example, some well-known diagnostic acute myelogenous leukemia, bladder cancer, breast cancer, chronic myelogenous leukemia, cervical cancer, lung cancer, prostate cancer or renal cell carcinoma markers in blood are as follows.
acute myelogenous leukemia; WT1
bladder cancer; SCC, TPA, or IAP
breast cancer; BCA225, TPA, CEA, IAP, or CA15-3
chronic myelogenous leukemia; BCR-ABL
cervical cancer; SCC, TPA, or CA125
lung cancer; AP, ACT, BFP, CA19-9, CA50, CA72-4, CA130, CEA, KMO-1, NSE, SCC, SP1, Span-1, TPA, CSLEX, SLX, STN and CYFRA
prostate cancer; PSA, or PAP
renal cell carcinoma; IAP

Namely, in this particular embodiment of the present invention, the outcome of the gene expression analysis serves as an intermediate result for further diagnosis of a subject's disease state.

In another embodiment, the present invention provides a method for detecting a diagnostic marker of cancer, said method comprising the step of detecting the expression of the JARID1B gene in a subject-derived biological sample as a diagnostic marker of acute myelogenous leukemia, bladder cancer, breast cancer, chronic myelogenous leukemia, cervical cancer, lung cancer, prostate cancer or renal cell carcinoma.

The present method comprises the step of

(a) determining the expression level of JARID1B gene in a subject-derived biological sample; and

(b) relating an increase in the expression level determined the step (a) as compared to a normal control level of the gene to the presence of cancerous cells.

A subject to be diagnosed by the present method is preferably a mammal. Exemplary mammals include, but are not limited to, e.g., human, non-human primate, mouse, rat, dog, cat, horse, and cow.

According to the present invention, the expression level of JARID1B gene in the subject-derived biological sample is determined. The expression level can be determined at the transcription product level, using methods known in the art. For example, the mRNA of JARID1B may be quantified using probes by hybridization methods (e.g., Northern hybridization). The detection may be carried out on a chip or an array. The use of an array is preferable for detecting the expression level of a plurality of genes (e.g., various cancer specific genes) including JARID1B gene. Those skilled in the art can prepare such probes utilizing the sequence information of JARID1B gene. Such sequence information can be obtained, but not limited to, from sequence databases on the Web, such as GeneBank™. For example, the exemplary sequence of JARID1B gene is, but not limited to, shown in SEQ ID NO: 1 (GenBank accession number: NM_{—}006618). Probes typically include at least 10, at least 20, at least 50, at least 100, at least 200 nucleotides of JARID1B gene sequence. Besides these fragments of JARID1B gene, the cDNA of JARID1B gene may be used as the probes. If necessary, the probe may be labeled with a suitable label, such as dyes, fluorescent and isotopes, and the expression level of the gene may be detected as the intensity of the hybridized labels.

Furthermore, the transcription product of JARID1B gene may be quantified using primers by amplification-based detection methods (e.g., RT-PCR). Such primers can also be prepared based on the sequence information of JARID1B gene. For example, the primers used in the Example (SEQ ID NOs: 7, 8, 9 and 10) may be employed for the detection by RT-PCR or Northern blot, but the present invention is not restricted thereto.

Specifically, a probe or primer used for the present method hybridizes under stringent, moderately stringent, or low stringent conditions to JARID1B mRNA. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degrees C. lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degrees C. for short probes or primers (e.g., 10 to 50 nucleotides) and at least about 60 degrees C. for longer probes or primers. Stringent conditions may also be achieved with the addition of destabilizing agents, such as formamide.

Alternatively, the translation product may be detected for the diagnosis of the present invention. For example, the quantity of JARID1B protein may be determined. A method for determining the quantity of the protein as the translation product includes immunoassay methods that use an antibody specifically recognizing the protein. The antibody may be monoclonal or polyclonal. Furthermore, any fragments or variants (e.g., chimeric antibody, scFv, Fab, F(ab′)2, Fv, etc.) of the antibody may be used for the detection, so long as they retain the binding ability to JARID1B protein. Methods to prepare these kinds of antibodies for the detection of the protein are well known in the art, and any method may be employed in the present invention to prepare such antibodies and equivalents thereof.

As another method to detect the expression level of JARID1B gene based on its translation product, the intensity of staining may be observed via immunohistochemical analysis using an antibody against JARID1B protein. Namely, the observation of strong staining indicates increased presence of the protein and at the same time high expression level of JARID1B gene.

Furthermore, the quantity of JARID1B protein can be determined by measuring the biological activity of JARID1B protein, such as histone demethylation. As described above, JARID1B has been characterized as one of the lysine demethylase families, which could remove the methyl group of histone H3 lysine 4 specifically (Yamane K. et al. Mol Cell 2007; 25: 801-12). Therefore, histone demethylation activity is useful for quantification of JARID1B protein based on its biological activity. The histone demethylation level can be determined by the methods well known in the art. When using histone demethylation activity for quantification of JARID1B protein, a homogenate, lysate or extract prepared from subject tissues or cells are preferably used as a biological sample, but not limited to. Biological sample may be incubated with methylated histone, and then demethylation level is estimated for example, by detecting formaldehyde or hydrogen peroxide release due to histone demethylation or residual methylated histone using antibody against methylated histone. Some commercial product may be available for histone demethylation assay, such as EpiQuik Global Histone Methylation Assay Kit (Epigentek Group Inc.).

Alternatively, cell proliferation activity may be used as a biological activity of JARID1B protein. According to the present invention, inhibiting the expression of JARID1B gene led to suppress the cell growth in bladder cancer and lung cancer cells, therefore, JARID1B protein is presumed to promote cell proliferation. For determining the cell proliferation activity of JARID1B protein, the cell is cultured in the presence of a biological sample, and then by detecting the speed of proliferation, or by measuring the cell cycle or the colony forming ability, the cell proliferating activity of the biological sample can be determined.

Moreover, in addition to the expression level of JARID1B gene, the expression level of other cancer-associated genes, for example, genes known to be differentially expressed in cancer may also be determined to improve the accuracy of the diagnosis.

The elevation in the expression level of JARID1B gene in a biological sample can be detected by comparison with a control level.

The control level may be determined at the same time with the test biological sample by using a sample(s) previously collected and stored from a subject/subjects whose disease state (cancerous or non-cancerous) is/are known. Alternatively, the control level may be determined by a statistical method based on the results obtained by analyzing previously determined expression level(s) of JARID1B gene in samples from subjects whose disease state are known. Furthermore, the control level can be a database of expression patterns from previously tested cells. Moreover, according to an aspect of the present invention, the expression level of JARID1B gene in a biological sample may be compared to multiple control levels, which control levels are determined from multiple reference samples. It is preferred to use a control level determined from a reference sample derived from a tissue type similar to that of the patient-derived biological sample. Moreover, it is preferred, to use the standard value of the expression levels of JARID1B gene in a population with a known disease state. The standard value may be obtained by any method known in the art. For example, a range of mean+/−2 S.D. or mean+/−3 S.D. may be used as standard value.

In the context of the present invention, a control level determined from a biological sample that is known not to be cancerous is referred to as a “normal control level”. On the other hand, if the control level is determined from a cancerous biological sample, it is referred to as a “cancerous control level”.

When the expression level of JARID1B gene is increased as compared to the normal control level or is similar to the cancerous control level, the biological sample may contain cancerous cells, which indicates that the subject may be suffering from or at a risk of developing cancer.

Difference between the expression levels of a test biological sample and the control level can be normalized to the expression level of a control gene, e.g., housekeeping genes, whose expression level is known not to differ depending on the cancerous or non-cancerous state of the cell. Exemplary control genes include, but are not limited to, beta-actin, glyceraldehyde 3 phosphate dehydrogenase, and ribosomal protein P1.

The expression level in a biological sample can be considered to be increased if it increases from the normal control level by, for example, 10%, 25% or 50%; or increases to more than 1.1 fold, more than 1.5 fold, more than 2.0 fold, more than 3.0 fold, more than 5.0 fold, more than 10.0 fold, or more. On the other hand, the expression level in a biological sample can be considered to be increased if the difference from cancerous control level is within 10%, 25% or 50%.

Kits for Detecting Cancer:

The present invention provides kits for detecting cancer, such as acute myelogenous leukemia, bladder cancer, chronic myelogenous leukemia, cervical cancer, lung cancer and renal cell carcinoma. The present reagent can be used for detecting cancerous cells in a biological sample, which may be useful for diagnosis of cancer.

Specifically, the reagent can detect the expression of JARID1B gene in a biological sample, which reagent may be selected from the group of:

(a) a reagent for detecting mRNA of JARID1B gene; and

(b) a reagent for detecting JARID1B protein.

(c) a reagent for detecting the biological activity of JARID1B protein.

Suitable reagents for detecting mRNA of JARID1B gene include oligonucleotides that specifically bind to or identify JARID1B mRNA, such as oligonucleotides which have a complementary sequence to a part of JARID1B mRNA. These kinds of oligonucleotides are exemplified by primers and probes that are specific to JARID1B mRNA. These kinds of oligonucleotides may be prepared based on methods well known in the art, for example as described in “Methods for diagnosing cancer”. If needed, the reagent for detecting JARID1B mRNA may be immobilized on a solid matrix.

On the other hand, suitable reagents for detecting JARID1B protein include antibodies to JARID1B protein. The antibody may be monoclonal or polyclonal. Furthermore, any fragments or variants (e.g., chimeric antibody, scFv, Fab, F(ab′)2, Fv, etc.) of the antibody may be used for the reagent, so long as they retain the binding ability to JARID1B protein. Methods to prepare these kinds of antibodies for the detection of JARID1B protein are well known in the art, and any method may be employed in the present invention to prepare such antibodies and equivalents thereof. Furthermore, the antibody may be labeled with signal generating molecules via direct linkage or an indirect labeling technique. Labels and methods for labeling antibodies and detecting the binding of antibodies to their targets are well known in the art and any labels and methods may be employed for the present invention. If needed, the reagent for detecting JARID1B protein may be immobilized on a solid matrix.

Furthermore, JARID1B protein can be detected as the biological activity of the JARID1B protein. The histone demethylation activity and cell proliferating activity are exemplary biological activities of JARID1B protein. The histone demethylation activity in a biological sample can be determined by detecting residual methylated histone using an antibody against methylated histone after incubating methylated histone with the biological sample. Thus, the present kit may include methylated histone and anti-methylated histone antibody. Otherwise, the present kit may include methylated histone with labeled methyl group for detecting formaldehyde released by histone demethylation. On the other hand, the cell proliferating activity in a biological sample can be determined by cultivating cells in the presence of the biological sample and then detecting the speed of proliferation, or measuring the cell cycle or the colony forming ability. Thus, the present kit can include medium and container for cultivation of cells.

The present kit may contain more than one of the aforementioned reagents. The kit may include a solid matrix and reagent for binding a probe against JARID1B gene or antibody against JARID1B, a medium and container for culturing cells, positive and negative control reagents, and a secondary antibody for detecting an antibody against JARID1B protein. A kit of the present invention may further include other materials desirable from a commercial and user standpoint, including buffers, diluents, filters, needles, syringes, and package inserts (e.g., written, tape, CD-ROM, etc.) with instructions for use. These reagents and such may be comprised in a container with a label. Suitable containers include bottles, vials, and test tubes. The containers may be formed from a variety of materials, such as glass or plastic.

As an embodiment of the present invention, when the reagent is a probe against JARID1B mRNA, the reagent may be immobilized on a solid matrix, such as a porous strip, to form at least one detection site. The measurement or detection region of the porous strip may include a plurality of sites, each containing a oligonucleotide (probe). A test strip may also contain sites for negative and/or positive controls. Alternatively, control sites may be located on a strip separated from the test strip. Optionally, the different detection sites may contain different amounts of immobilized oligonucleotides, i.e., a higher amount in the first detection site and lesser amounts in subsequent sites. Upon the addition of test sample, the number of sites displaying a detectable signal provides a quantitative indication of the amount of JARID1B mRNA present in the sample. The detection sites may be configured in any suitably detectable shape and are typically in the shape of a bar or dot spanning the width of a test strip.

The kit of the present invention may further include a positive control sample or JARID1B standard sample. The positive control sample of the present invention may be prepared by collecting jARID1B positive samples such as cells derived from acute myelogenous leukemia, bladder cancer, chronic myelogenous leukemia, cervical cancer, lung cancer and renal cell carcinoma, and then those JARID1B level are assayed. Alternatively, purified JARID1B protein or JARID1B gene may be transfected to cell to form the positive sample or JARID1B standard.

In another embodiment, the kit of the present invention may further include a negative control sample. The negative control sample of the present invention is non-JARID1B expressing cells or tissue such as non-cancerous cells.

Double-Stranded Molecules:

As used herein, the term “isolated double-stranded molecule” refers to a nucleic acid molecule that inhibits expression of a target gene and includes, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)).

As used herein, sense strand of a target sequence is a nucleotide sequence within mRNA or cDNA sequence of a gene, which will result in suppress of translation of the whole mRNA if a double-stranded nucleic acid molecule of the invention was introduced within a cell expressing the gene. A nucleotide sequence within mRNA or cDNA sequence of a gene can be determined to be a target sequence when a double-stranded polynucleotide comprising a sequence corresponding to the target sequence inhibits expression of the gene in a cell expressing the gene. The double stranded polynucleotide by which suppresses the gene expression may consists of the target sequence and 3′overhang having 2 to 5 nucleotides in length (e.g., uu).

As used herein, the term “siRNA” refers to a double-stranded RNA molecule which prevents translation of a target mRNA. Standard techniques of introducing siRNA into the cell are used, including those in which DNA is a template from which RNA is transcribed. The siRNA includes a part of sense nucleic acid sequence of the target gene (also referred to as “sense strand”), a part of antisense nucleic acid sequence of the target gene (also referred to as “antisense strand”) or both. The siRNA may be constructed such that a single transcript has both the sense and complementary antisense nucleic acid sequences of the target gene, e.g., a hairpin. The siRNA may either be a dsRNA or shRNA.

As used herein, the term “dsRNA” refers to a construct of two RNA molecules composed of complementary sequences to one another and that have annealed together via the complementary sequences to form a double-stranded RNA molecule. The nucleotide sequence of two strands may include not only the “sense” or “antisense” RNAs selected from a protein coding sequence of target gene sequence, but also RNA molecule having a nucleotide sequence selected from non-coding region of the target gene.

The term “shRNA”, as used herein, refers to an siRNA having a stem-loop structure, composed of first and second regions complementary to one another, i.e., sense and antisense strands. The degree of complementarity and orientation of the regions are sufficient such that base pairing occurs between the regions, the first and second regions are joined by a loop region, and the loop results from a lack of base pairing between nucleotides (or nucleotide analogs) within the loop region. The loop region of an shRNA is a single-stranded region intervening between the sense and antisense strands and may also be referred to as “intervening single-strand”.

As use herein, the term “siD/R-NA” refers to a double-stranded nucleic acid molecule which is composed of both RNA and DNA, and includes hybrids and chimeras of RNA and DNA and prevents translation of a target mRNA. Herein, a hybrid indicates a molecule wherein a polynucleotide composed of DNA and a polynucleotide composed of RNA hybridize to each other to form the double-stranded molecule; whereas a chimera indicates that one or both of the strands composing the double stranded molecule may contain RNA and DNA. Standard techniques of introducing siD/R-NA into the cell are used. The siD/R-NA includes a part of sense nucleic acid sequence of the target gene (also referred to as “sense strand”), a part of antisense nucleic acid sequence of the target gene (also referred to as “antisense strand”) or both. The siD/R-NA may be constructed such that a single transcript has both the sense and complementary antisense nucleic acid sequences from the target gene, e.g., a hairpin. The siD/R-NA may either be a dsD/R-NA or shD/R-NA.

As used herein, the term “dsD/R-NA” refers to a construct of two molecules composed of complementary sequences to one another and that have annealed together via the complementary sequences to form a double-stranded polynucleotide molecule. The nucleotide sequence of two strands may comprise not only the “sense” or “antisense” nucleic acid sequence selected from a protein coding sequence of target gene sequence, but also polynucleotide having a nucleic acid sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, composed of a first and second regions complementary to one another, i.e., sense and antisense strands. The degree of complementarity and orientation of the regions are sufficient such that base pairing occurs between the regions, the first and second regions are joined by a loop region, and the loop resulting from a lack of base pairing between nucleotides (or nucleotide analogs) within the loop region. The loop region of an shD/R-NA is a single-stranded region intervening between the sense and antisense strands and may also be referred to as “intervening single-strand”.

As used herein, the term “isolated” is a state removed from its original environment (e.g., the natural environment if naturally occurring) and thus, synthetically altered from its natural state.

A double-stranded molecule against the JARID1B gene, which molecule hybridizes to JARID1B mRNA, decreases or inhibits production of JARID1B protein by associating with the normally single-stranded mRNA transcript of the gene, thereby interfering with translation and thus, inhibiting expression of the protein. As demonstrated herein, the expression of JARID1B gene in bladder cancer cell lines and lung cancer cell lines was inhibited by dsRNA (FIG. 7B, F) and consequently, the growths of those cell lines were suppressed (FIG. 7C, G).

Therefore the present invention provides isolated double-stranded molecules that are capable of inhibiting the expression of JARID1B gene as well as the cell proliferation when introduced into a cell expressing the gene. The double-stranded molecules of the present invention are useful for inhibiting cancer cell proliferation relating to the over-expression of JARID1B gene, therefore, they may provide new methods for treating cancers. For example, the double-stranded molecules of the present invention are suitable for applying to cancers such as acute myelogenous leukemia, bladder cancer, breast cancer, chronic myelogenous leukemia, cervical cancer, lung cancer, prostate cancer and renal cell carcinoma, in which the overexpression of JARID1B gene was observed (Table 3). More preferably, bladder cancer and lung cancer are suitable for the present double-stranded molecule.

Methods for designing double-stranded molecules having the ability to inhibit target gene expression in cells are known. (See, for example, WO2004/048566 and U.S. Pat. No. 6,506,559, herein incorporated by reference in their entirety). For example, a computer program for designing siRNAs is available from the Ambion website (www.ambion.com/techlib/misc/siRNA_finder.html).

The computer program selects target nucleotide sequences for double-stranded molecules based on the following protocol.

Selection of Target Sites:

1. Beginning with the AUG start codon of the transcript, scan downstream for AA dinucleotide sequences. Record the occurrence of each AA and the 3′ adjacent 19 nucleotides as potential siRNA target sites. Tuschl et al. recommend to avoid designing siRNA to the 5′ and 3′ untranslated regions (UTRs) and regions near the start codon (within 75 bases) as these may be richer in regulatory protein binding sites, and UTR-binding proteins and/or translation initiation complexes may interfere with binding of the siRNA endonuclease complex.

2. Compare the potential target sites to the appropriate genome database (human, mouse, rat, etc.) and eliminate from consideration any target sequences with significant homology to other coding sequences. Basically, BLAST, which can be found on the NCBI server at: www.ncbi.nlm.nih.gov/BLAST/, is used (Altschul S F et al., Nucleic Acids Res Sep. 1, 1997, 25(17): 3389-402).

3. Select qualifying target sequences for synthesis. Selecting several target sequences along the length of the gene to evaluate is typical.

Using the above designing method, the target sequence of the isolated double-stranded molecules of the present invention was designed as SEQ ID NOs: 21 and 30. Double-stranded molecules targeting the above-mentioned target sequence were examined for their ability to suppress the growth of cells expressing JARID1B gene. Therefore, the exemplary double-stranded molecule of the present invention includes the nucleic acid sequence corresponding to SEQ ID NOs: 21 or 30 as a target sequence. However, any of sequences from JARID1B can be a target sequence for the double-stranded molecule of the present invention so long as the double-stranded molecule retains the ability to inhibit the expression of JARID1B and the cell proliferation.

The double-stranded molecule of the present invention may be directed to a single target sequence.

The double-stranded molecule of the present invention includes isolated polynucleotides that comprises any of the nucleic acid sequences of target sequences and/or complementary sequences to the target sequences. Examples of the polynucleotide for the present double-stranded molecule include those comprising the nucleic acid sequence corresponding to SEQ ID NO: 21 or 30 and/or complementary sequence to the sequence. However, the present invention is not limited to these examples, and minor modifications in the aforementioned nucleic acid sequences are acceptable so long as the modified molecule retains the ability to suppress the expression of JARID1B gene. Herein, the phrase “minor modification” as used in connection with a nucleic acid sequence indicates one, two or several substitution, deletion, addition or insertion of nucleic acids to the sequence. In the context of the present invention, the term “several” as applies to nucleic acid substitutions, deletions, additions and/or insertions may mean 3-7, preferably 3-5, more preferably 3-4, even more preferably 3 nucleic acid residues.

When the isolated polynucleotide for the present double stranded-molecule consists of RNA or derivatives thereof, base “t” should be replaced with “u” in the nucleotide sequences. As used herein, the term “complementary” refers to Watson-Crick or Hoogsteen base pairing between nucleotides units of a polynucleotide, and the term “binding” means the physical or chemical interaction between two polynucleotides. When the polynucleotide includes modified nucleotides and/or non-phosphodiester linkages, these polynucleotides may also bind each other as same manner. Generally, complementary polynucleotide sequences hybridize under appropriate conditions to form stable duplexes containing few or no mismatches. Furthermore, a sense strand and an antisense strand complementary thereto can form double-stranded molecule or hairpin loop structure by the hybridization. In a preferred embodiment, such duplexes contain no more than 1 mismatch for every 10 matches. In an especially preferred embodiment, where the strands of the duplex are fully complementary, such duplexes contain no mismatches.

The double-stranded molecule is preferably less than 6393 nucleotides in length for JARID1B gene. For example, the double-stranded molecule is less than 500, 200, 100, 75, 50, or 25 nucleotides in length. Further, sense strand of the double-stranded molecule may be longer than 19 nucleotides, preferably longer than 21 nucleotides, and more preferably has a length of between about 19 and 25 nucleotides. Accordingly, the present invention provides the double-stranded molecules comprising a sense strand and an antisense strand, wherein the sense strand comprises a nucleotide sequence corresponding to a target sequence. In preferable embodiments, the sense strand hybridizes with antisense strand at the target sequence to form the double-stranded molecule having between 19 and 25 nucleotide pair in length.

According to the present invention, a double-stranded molecule of the present invention can be tested for its ability using the methods utilized in the Examples. For example, double-stranded molecules composed of sense strand of a portion of mRNA of JARID1B gene and antisense strand complementary thereto were tested in vitro for their ability to decrease production of JARID1B mRNA in bladder cancer and lung cancer cell lines (e.g., 253J, 253J-BV, HT1197, HT1376, J82, SCaBER, UMUC3, EJ28, RT4, T24 and SW780 for bladder cancer; SBC5, A549, H2170, LC319, and RERF-LC-AI for lung cancer) according to standard methods. Furthermore, for example, reduction in JARID1B mRNA in cells contacted with the candidate a double-stranded molecule compared to cells cultured in the absence of the candidate molecule can be detected by, e.g. RT-PCR using primers for JARID1B mRNA mentioned under Example 1 item “Quantitative real-time PCR”. Sequences which decrease the production of JARID1B mRNA in in vitro cell-based assays can then be tested for there inhibitory effects on cell growth. Sequences which inhibit cell growth in in vitro cell-based assay can then be tested for their in vivo ability using animals with cancer, e.g. nude mouse xenograft models, to confirm decreased production of JARID1B mRNA and decreased cancer cell growth.

The double-stranded molecule serves as a guide for identifying homologous sequences in mRNA for the RISC complex, when the double-stranded molecule is introduced into cells. The identified target RNA is cleaved and degraded by the nuclease activity of Dicer, through which the double-stranded molecule eventually decreases or inhibits production (expression) of the polypeptide encoded by the RNA. Thus, a double-stranded molecule of the present invention can be defined by its ability to generate a single-strand that specifically hybridizes to the mRNA of the JARID1B gene under stringent conditions. Herein, the portion of the mRNA that hybridizes with the single-strand generated from the double-stranded molecule is referred to as “target sequence” or “target nucleic acid” or “target nucleotide”. In the present invention, nucleotide sequence of the “target sequence” can be shown using not only the RNA sequence of the mRNA, but also the DNA sequence of cDNA synthesized from the mRNA.

The double-stranded molecules of the present invention may contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which may be incorporated into the present molecules (WO03/070744; WO2005/045037). In one embodiment, modifications can be used to provide improved resistance to degradation or improved uptake. Examples of such modifications include, but are not limited to, phosphorothioate linkages, 2′-O-methyl ribonucleotides (especially on the sense strand of a double-stranded molecule), 2′-deoxy-fluoro ribonucleotides, 2′-deoxy ribonucleotides, “universal base” nucleotides, 5′-C-methyl nucleotides, and inverted deoxybasic residue incorporation (US20060122137).

In another embodiment, modifications can be used to enhance the stability or to increase targeting efficiency of the double-stranded molecule. Examples of such modifications include, but are not limited to, chemical cross linking between the two complementary strands of a double-stranded molecule, chemical modification of a 3′ or 5′ terminus of a strand of a double-stranded molecule, sugar modifications, nucleobase modifications and/or backbone modifications, 2 -fluoro modified ribonucleotides and 2′-deoxy ribonucleotides (WO2004/029212). In another embodiment, modifications can be used to increased or decreased affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deaza, 7-alkyl, or 7-alkenyl purine.

In another preferable embodiment, the present double-stranded molecules have 3′ over hang consisting of a few nucleotides at either or both of the sense strand and/or the antisense strand 3′ terminal. when the double-stranded molecule is a double-stranded molecule with a 3′ overhang, the 3′ overhanging nucleotides may be replaced by deoxyribonucleotides (Elbashir S M et al., Genes Dev Jan. 15, 2001, 15(2): 188-200). Preferably, the 3′ overhang for the present double-stranded molecule consists of two deoxyribonucleotides “t”. For example, the exemplary sequence for the present double-stranded molecule with 3′ overhang is shown in SEQ ID NOs: 19 or 28 for the sense strand and SEQ ID NOs: 20 or 29 for the antisense strand, which include the target sequence corresponding to SEQ ID NOs: 21 or 30 and 3′ overhang sequence consisting of two deoxyribonucleotides “t”. For further details, published documents such as US20060234970 are available.

The present invention is not limited to these examples and any known chemical modifications may be employed for the double-stranded molecules of the present invention so long as the resulting molecule retains the ability to inhibit the expression of the target gene.

Furthermore, the double-stranded molecules of the present invention may comprise both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule composed of a DNA strand (polynucleotide) and an RNA strand (polynucleotide), a chimera type double-stranded molecule containing both DNA and RNA on any or both of the single strands (polynucleotides), or the like may be formed for enhancing stability of the double-stranded molecule.

The hybrid of a DNA strand and an RNA strand may be either where the sense strand is DNA and the antisense strand is RNA, or the opposite so long as it can inhibit expression of the target gene when introduced into a cell expressing the gene. Preferably, the sense strand polynucleotide is DNA and the antisense strand polynucleotide is RNA. Also, the chimera type double-stranded molecule may be either where both of the sense and antisense strands are composed of DNA and RNA, or where any one of the sense and antisense strands is composed of DNA and RNA so long as it has an activity to inhibit expression of the target gene when introduced into a cell expressing the gene. In order to enhance stability of the double-stranded molecule, the molecule preferably contains as much DNA as possible, whereas to induce inhibition of the target gene expression, the molecule is required to be RNA within a range to induce sufficient inhibition of the expression.

As a preferred example of the chimera type double-stranded molecule, an upstream partial region (i.e., a region flanking to the target sequence or complementary sequence thereof within the sense or antisense strands) of the double-stranded molecule is RNA. Preferably, the upstream partial region indicates the 5′ side (5′-end) of the sense strand and the 3′ side (3′-end) of the antisense strand. Alternatively, regions flanking to 5′-end of sense strand and/or 3′-end of antisense strand are referred to upstream partial region. That is, in preferable embodiments, a region flanking to the 3′-end of the antisense strand, or both of a region flanking to the 5′-end of sense strand and a region flanking to the 3′-end of antisense strand are composed of RNA. For instance, the chimera or hybrid type double-stranded molecule of the present invention include following combinations.

sense strand:
5′-[---DNA---]-3′
3′-(RNA)-[DNA]-5′:
antisense strand,
sense strand:
5′-(RNA)-[DNA]-3′
3′-(RNA)-[DNA]-5′:
antisense strand,
and
sense strand:
5′-(RNA)-[DNA]-3′
3′-(---RNA---)-5′:
antisense strand.

The upstream partial region preferably is a domain composed of 9 to 13 nucleotides counted from the terminus of the target sequence or complementary sequence thereto within the sense or antisense strands of the double-stranded molecules. Moreover, preferred examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded molecule, the effect to inhibit expression of the target gene is much higher when the entire antisense strand is RNA (US20050004064).

In the present invention, the double-stranded molecule may form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA comprises the sense target sequence and the antisense target sequence on a single polynucleotide wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

A loop sequence composed of an arbitrary nucleotide sequence can be located between the sense and antisense sequence in order to form the hairpin loop structure. Thus, the present invention also provides a double-stranded molecule having the general formula 5′-[A]-[B]-[A′]-3′, wherein [A] is the sense strand containing a sequence corresponding to a target sequence, [B] is an intervening single-strand and [A′] is the antisense strand containing a complementary sequence to [A]. The target sequence may be, for example, the sequence corresponding to SEQ ID NO: 21 or 30.

The present invention is not limited to these examples, and the target sequence in [A] may be modified sequences from these examples so long as the double-stranded molecule retains the ability to suppress the expression of JARID1Bgene. The region [A] hybridizes to [A′] to form a loop composed of the region [B]. The intervening single-stranded portion [B], i.e., loop sequence may be preferably 3 to 23 nucleotides in length. The loop sequence, for example, can be selected from among the following sequences (www.ambion.com/techlib/tb/tb_{—}506.html). Furthermore, loop sequence consisting of 23 nucleotides also provides active siRNA (Jacque J M et al., Nature Jul. 25, 2002, 418(6896): 435-8, Epub Jun. 26, 2002):

CCC, CCACC, or CCACACC: Jacque J M et al., Nature Jul. 25, 2002, 418(6896): 435-8, Epub Jun. 26, 2002;

UUCG: Lee N S et al., Nat Biotechnol May 2002, 20(5): 500-5; Fruscoloni P et al., Proc Natl Acad Sci USA Feb. 18, 2003, 100(4): 1639-44, Epub Feb. 10, 2003; and

UUCAAGAGA: Dykxhoorn D M et al., Nat Rev Mol Cell Biol June 2003, 4(6): 457-67.

Examples of preferred double-stranded molecules of the present invention having hairpin loop structure are shown below. In the following structure, the loop sequence can be selected from among AUG, CCC, UUCG, CCACC, CTCGAG, AAGCUU, CCACACC, and UUCAAGAGA; however, a preferred example of the present invention is not limited thereto:

(SEQ ID NO: 31)
CAGUGAAUGAGCUCCGGCA
(SEQ ID NO: 32)
-[B]- UGCCGGAGCUCAUUCACUG
(for target sequence SEQ ID NO: 21).

Another preferred example of the present invention is:

(SEQ ID NO: 33)
GGAAUAUGGAGCUGACAUU
(SEQ ID NO: 34)
[B]- AAUGUCAGCUCCAUAUUCC
(for target sequence SEQ ID NO: 30).

Furthermore, in order to enhance the inhibition activity of the double-stranded molecules, a few nucleotides can be added to 3′end of the sense strand and/or antisense strand of the target sequence, as 3′ overhangs. The number of nucleotides to be added is at least 2, generally 2 to 10, preferably 2 to 3. The kind of nucleotides are not limited, but preferably “u” or “t”. The added nucleotides form single strand at the 3′end of the sense strand and/or antisense strand of the double-stranded molecule.

When the double-stranded molecule has a hairpin loop structure, a 3′ overhang is added to the 3′ end of the antisense strand. Preferred examples of such double-stranded molecules include, but are not limited to:

(SEQ ID NO: 31)
CAGUGAAUGAGCUCCGGCA
(SEQ ID NO: 20)
-[B]- UGCCGGAGCUCAUUCACUGTT
(for target sequence SEQ ID NO: 21)
and
(SEQ ID NO: 33)
GGAAUAUGGAGCUGACAUU
(SEQ ID NO: 29)
-[B]- AAUGUCAGCUCCAUAUUCCTT
(for target sequence SEQ ID NO: 30).

The method for preparing the double-stranded molecule is not particularly limited though it is preferable to use a chemical synthetic method known in the art. According to the chemical synthesis method, sense and antisense single-stranded polynucleotides are separately synthesized and then annealed together via an appropriate method to obtain a double-stranded molecule. Specific example for the annealing includes wherein the synthesized single-stranded polynucleotides are mixed in a molar ratio of preferably at least about 3:7, more preferably about 4:6, and most preferably substantially equimolar amount (i.e., a molar ratio of about 5:5). Next, the mixture is heated to a temperature at which double-stranded molecules dissociate and then is gradually cooled down. The annealed double-stranded polynucleotide can be purified by usually employed methods known in the art. Example of purification methods include methods utilizing agarose gel electrophoresis or wherein remaining single-stranded polynucleotides are optionally removed by, e.g., degradation with appropriate enzyme.

The regulatory sequences flanking JARID1B sequences may be identical or different, such that their expression can be modulated independently, or in a temporal or spatial manner. The double-stranded molecules can be transcribed intracellularly by cloning JARID1B gene templates into a vector containing, e.g., a RNA pol III transcription unit from the small nuclear RNA (snRNA) U6 or the human H1 RNA promoter.

Vectors Encoding a Double-Stranded Molecule of the Present Invention:

Also included in the present invention are vectors encoding one or more of the double-stranded molecules described herein, and a cell containing such a vector. A vector of the present invention preferably comprises a nucleic acid sequence encoding a double-stranded molecule of the present invention in an expressible form. Herein, the phrase “in an expressible form” indicates that the vector, when introduced into a cell, will express the molecule. In a preferred embodiment, the vector includes regulatory elements necessary for expression of the double-stranded molecule. Such vectors of the present invention may be used for producing the double-stranded molecules of the present invention, or directly as an active ingredient for treating cancer.

Alternatively, the present invention provides vectors including each of a combination of polynucleotide having a sense strand nucleic acid and an antisense strand nucleic acid, wherein said sense strand nucleic acid includes nucleotide sequence of SEQ ID NOs: 21 or 30, and said antisense strand nucleic acid consists of a sequence complementary to the sense strand, wherein the transcripts of said sense strand and said antisense strand hybridize to each other to form a double-stranded molecule, and wherein said vectors, when introduced into a cell expressing the JARID1B, inhibits expression of said gene. Preferably, the polynucleotide is an oligonucleotide of between about 19 and 25 nucleotides in length (e.g., contiguous nucleotides from the nucleotide sequence of SEQ ID NO: 1. More preferably, the combination of polynucleotide includes a single nucleotide transcript having the sense strand and the antisense strand linked via a single-stranded nucleotide sequence. More preferably, the combination of polynucleotide has the general formula 5′-[A]-[B]-[A′]-3′, wherein [A] is a nucleotide sequence including SEQ ID NOs: 21 or 30; [B] is a nucleotide sequence consisting of about 3 to about 23 nucleotide; and [A′] is a nucleotide sequence complementary to [A].

Vectors of the present invention can be produced, for example, by cloning JARID1B sequence into an expression vector so that regulatory sequences are operatively-linked to JARID1B sequence in a manner to allow expression (by transcription of the DNA molecule) of both strands (Lee N S et al., Nat Biotechnol May 2002, 20(5): 500-5). For example, RNA molecule that is the antisense strand to mRNA is transcribed by a first promoter (e.g., a promoter sequence flanking to the 3′ end of the cloned DNA) and RNA molecule that is the sense strand to the mRNA is transcribed by a second promoter (e.g., a promoter sequence flanking to the 5′ end of the cloned DNA). The sense and antisense strands hybridize in vivo to generate a double-stranded molecule constructs for silencing of the gene. Alternatively, two vectors constructs respectively encoding the sense and antisense strands of the double-stranded molecule are utilized to respectively express the sense and anti-sense strands and then forming a double-stranded molecule construct. Furthermore, the cloned sequence may encode a construct having a secondary structure (e.g., hairpin); namely, a single transcript of a vector contains both the sense and complementary antisense sequences of the target gene.

The vectors of the present invention may also be equipped so as to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivacaine, polymers, peptide-mediated) delivery, cationic lipid complexes, and particle-mediated (“gene gun”) or pressure-mediated delivery (see, e.g., U.S. Pat. No. 5,922,687).

The vectors of the present invention include, for example, viral or bacterial vectors. Examples of expression vectors include attenuated viral hosts, such as vaccinia or fowlpox (see, e.g., U.S. Pat. No. 4,722,848). This approach involves the use of vaccinia virus, e.g., as a vector to express nucleotide sequences that encode the double-stranded molecule. Upon introduction into a cell expressing the target gene, the recombinant vaccinia virus expresses the molecule and thereby suppresses the proliferation of the cell. Another example of useable vector includes Bacille Calmette Guerin (BCG). BCG vectors are described in Stover et al., Nature 1991, 351: 456-60. A wide variety of other vectors are useful for therapeutic administration and production of the double-stranded molecules; examples include adeno and adeno-associated virus vectors, retroviral vectors, Salmonella typhi vectors, detoxified anthrax toxin vectors, and the like. See, e.g., Shata et al., Mol Med Today 2000, 6: 66-71; Shedlock et al., J Leukoc Biol 2000, 68: 793-806; and Hipp et al., In Vivo 2000, 14: 571-85.

Methods of Inhibiting or Reducing Growth of a Cancer Cell and Treating Cancer Using a Double-Stranded Molecule of the Present Invention:

In the present invention, a dsRNA were tested for their ability to inhibit cell growth. The dsRNA effectively knocked down the expression of the gene in bladder cancer cell lines and lung cancer cell lines coincided with suppression of cell proliferation (FIG. 7).

Therefore, the present invention provides methods for inhibiting cell growth, i.e., cancer cell growth, by inducing dysfunction of JARID1B gene via inhibiting the expression of JARID1B gene. According to the present invention, the expression of JARID1B gene was specifically elevated in acute myelogenous leukemia, bladder cancer, breast cancer, chronic myelogenous leukemia, cervical cancer, lung cancer, prostate cancer and renal cell carcinoma (Table 3). Therefore, the methods of the present invention may be useful for inhibiting cell growth in such caners. JARID1B gene expression can be inhibited by any of the aforementioned double-stranded molecules of the present invention which specifically target JARID1B gene or the vectors of the present invention that can express any of the double-stranded molecules. For example, the double-stranded molecule comprising the nucleic acid sequence corresponding to SEQ ID NOs: 21 or 30 as a target sequence can be preferably used for the present method.

Such ability of the present double-stranded molecules and vectors to inhibit cell growth of cancerous cell demonstrates that they can be used for methods for treating aforementioned cancers. Thus, the present invention provides methods to treat patients with cancer by administering a double-stranded molecule against JARID1B gene or a vector expressing the molecule.

The growth of cells expressing JARID1B gene may be inhibited by contacting the cells with a double-stranded molecule against JARID1B gene, a vector expressing the molecule or a composition containing the same. The cell may be further contacted with a transfection agent. Suitable transfection agents are known in the art. The phrase “inhibition of cell growth” indicates that the cell proliferates at a lower rate or has decreased viability as compared to a cell not exposed to the molecule. Cell growth may be measured by methods known in the art, e.g., using the MTT cell proliferation assay.

The growth of any kind of cell may be suppressed according to the present method so long as the cell expresses or over-expresses JARID1B gene. Exemplary cells include acute myelogenous leukemia, bladder cancer, breast cancer, chronic myelogenous leukemia, cervical cancer, lung cancer, prostate cancer and renal cell carcinoma, especially, bladder cancer and lung cancer.

Thus, patients suffering from or at risk of developing disease related to JARID1B may be treated by administering at least one of double-stranded molecules of the present invention, at least one vector expressing at least one of the molecules or at least one composition containing at least one of the molecules. For example, patients of lung cancer or bladder cancer may be treated according to the present methods. Types of cancers may be identified by standard methods according to the particular type of tumor to be diagnosed. For example, lung cancer may be diagnosed with CEA, CYFRA and so on, as lung cancer marker, or with Chest X-Ray and/or Sputum Cytology. Bladder cancer may be diagnosed with urine analysis, X-ray analysis or cystoscope inspection. More preferably, patients to be treated by the methods of the present invention may be selected by detecting the expression level of JARID1B in a sample collected from the patient using conventional methods such as RT-PCR and immunoassay. Preferably, before the treatment of the present invention, a biopsy specimen from the subject is confirmed for JARID1B gene overexpression using methods known in the art, for example, immunohistochemical analysis or RT-PCR.

According to the method of the present invention, plural kinds of the double-stranded molecules against JARID1B gene (or vectors expressing or compositions containing the same) can be used for administration.

For inhibiting cell growth, a double-stranded molecule of present invention may be directly introduced into the cells in a form to achieve binding of the molecule with corresponding mRNA transcripts. Alternatively, as described above, a DNA encoding the double-stranded molecule may be introduced into cells as a vector. For introducing the double-stranded molecules and vectors into the cells, transfection-enhancing agent, such as FuGENE (Roche diagnostics), Lipofectamine 2000 (Invitrogen), Oligo-fectamine (Invitrogen), and Nucleofector (Wako pure Chemical), may be employed.

A treatment is deemed “efficacious” if it leads to clinical benefit such as, reduction in expression of JARID1B gene, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

It is understood that the double-stranded molecule of the present invention degrades JARID1B mRNA in substoichiometric amounts. Without wishing to be bound by any theory, it is believed that the double-stranded molecule of the present invention causes degradation of the target mRNA in a catalytic manner. Thus, compared to standard cancer therapies, significantly less a double-stranded molecule needs to be delivered at or near the site of cancer to exert therapeutic effect.

One skilled in the art can readily determine an effective amount of the double-stranded molecule of the present invention to be administered to a given subject, by taking into account factors such as body weight, age, sex, type of disease, symptoms and other conditions of the subject; the route of administration; and whether the administration is regional or systemic. Generally, an effective amount of the double-stranded molecule of the present invention is an intercellular concentration at or near the cancer site of from about 1 nanomolar (nM) to about 100 nM, preferably from about 2 nM to about 50 nM, more preferably from about 2.5 nM to about 10 nM. It is contemplated that greater or smaller amounts of the double-stranded molecule can be administered. The precise dosage required for a particular circumstance may be readily and routinely determined by one of skill in the art.

The present methods can be used to inhibit the growth or metastasis of cancer expressing JARID1B gene; for example acute myelogenous leukemia, bladder cancer, breast cancer, chronic myelogenous leukemia, cervical cancer, lung cancer, prostate cancer and renal cell carcinoma, especially bladder cancer and lung cancer. In particular, a double-stranded molecule containing a target sequence corresponding to SEQ ID NOs: 21 or 30 is particularly preferred for the treatment of cancer.

For treating cancer, the double-stranded molecule of the invention can also be administered to a subject in combination with a pharmaceutical agent different from the double-stranded molecule. Alternatively, the double-stranded molecule of the present invention can be administered to a subject in combination with another therapeutic method designed to treat cancer. For example, the double-stranded molecule of the present invention can be administered in combination with therapeutic methods currently employed for treating cancer or preventing cancer metastasis (e.g., radiation therapy, surgery and treatment using chemotherapeutic agents).

In the present methods, the double-stranded molecule can be administered to the subject either as a naked double-stranded molecule, in conjunction with a delivery reagent, or as a recombinant plasmid or viral vector which expresses the double-stranded molecule.

Suitable delivery reagents for administration in conjunction with a double-stranded molecule of the present invention include the Minis Transit TKO lipophilic reagent; lipofectin; lipofectamine; cellfectin; or polycations (e.g., polylysine), or liposomes. A preferred delivery reagent is a liposome.

Liposomes can aid in the delivery of the double-stranded molecule to a particular tissue, such as retinal or tumor tissue, and can also increase the blood half-life of the double-stranded molecule. Liposomes suitable for use in the method of the present invention are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al., Ann Rev Biophys Bioeng 1980, 9: 467; and U.S. Pat. Nos. 4,235,871; 4,501,728; 4,837,028; and 5,019,369, the entire disclosures of which are herein incorporated by reference.

In some embodiments, the liposomes encapsulating the present double-stranded molecule comprise a ligand molecule that can deliver the liposome to the cancer site. Ligands which bind to receptors prevalent in tumor or vascular endothelial cells, such as monoclonal antibodies that bind to tumor antigens or endothelial cell surface antigens, are preferred.

In addition, the liposomes encapsulating the present double-stranded molecule can be modified so as to avoid clearance by the mononuclear macrophage and reticuloendothelial systems, for example, by having opsonization-inhibition moieties bound to the surface of the structure. In one embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example, solid tumors, will efficiently accumulate these liposomes; see Gabizon et al., Proc Natl Acad Sci USA 1988, 18: 6949-53. In addition, the reduced uptake by the RES lowers the toxicity of stealth liposomes by preventing significant accumulation in liver and spleen. Thus, liposomes of the invention that are modified with opsonization-inhibition moieties can deliver the present double-stranded molecule to tumor cells.

Opsonization inhibiting moieties suitable for modifying liposomes are preferably water-soluble polymers with a molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM.sub.1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

Preferably, the opsonization-inhibiting moiety is a PEG, PPG, or derivatives thereof. Liposomes modified with PEG or PEG-derivatives are sometimes called “PEGylated liposomes”.

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH.sub.3 and a solvent mixture such as tetrahydrofuran and water in a 30:12 ratio at 60 degrees C.

Vectors expressing a double-stranded molecule of the present invention are discussed above. Such vectors expressing at least one double-stranded molecule of the present invention can also be administered directly or in conjunction with a suitable delivery reagent, including the Mirus Transit LT1 lipophilic reagent; lipofectin; lipofectamine; cellfectin; polycations (e.g., polylysine) or liposomes. Methods for delivering recombinant viral vectors, which express a double-stranded molecule of the present invention, to an area of cancer in a patient are within the skill of the art.

The double-stranded molecule of the present invention can be administered to the subject by any means suitable for delivering the double-stranded molecule into cancer sites. For example, the double-stranded molecule can be administered by gene gun, electroporation, or by other suitable parenteral or enteral administration routes.

Suitable enteral administration routes include oral, rectal, or intranasal delivery.

Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection); subcutaneous injection or deposition including subcutaneous infusion (such as by osmotic pumps); direct application to the area at or near the site of cancer, for example by a catheter or other placement device (e.g., a suppository or an implant comprising a porous, non-porous, or gelatinous material); and inhalation. It is preferred that injections or infusions of the double-stranded molecule or vector be given at or near the site of cancer.

The double-stranded molecule of the present invention can be administered in a single dose or in multiple doses. Where the administration of the double-stranded molecule of the present invention is by infusion, the infusion can be a single sustained dose or can be delivered by multiple infusions. Injection of the agent directly into the tissue is at or near the site of cancer preferred. Multiple injections of the agent into the tissue at or near the site of cancer are particularly preferred.

One skilled in the art can also readily determine an appropriate dosage regimen for administering the double-stranded molecule of the present invention to a given subject. For example, the double-stranded molecule can be administered to the subject once, for example, as a single injection or deposition at or near the cancer site. Alternatively, the double-stranded molecule can be administered once or twice daily to a subject for a period of from about three to about twenty-eight days, more preferably from about seven to about ten days. In a preferred dosage regimen, the double-stranded molecule is injected at or near the site of cancer once a day for seven days. Where a dosage regimen comprises multiple administrations, it is understood that the effective amount of a double-stranded molecule administered to the subject can comprise the total amount of a double-stranded molecule administered over the entire dosage regimen.

Compositions Containing a Double-Stranded Molecule of the Present Invention:

In addition to the above, the present invention also provides pharmaceutical compositions that include at least one of the double-stranded molecules of the present invention or the vectors encoding thereof. The pharmaceutical compositions inhibit the expression of JARID1B gene and consequently suppress cancer cell growth, therefore, they may be useful for treating or preventing cancer relating to overexpression of the JARID1B gene, for example, acute myelogenous leukemia, bladder cancer, breast cancer, chronic myelogenous leukemia, cervical cancer, lung cancer, prostate cancer and renal cell carcinoma. Preferably, the pharmaceutical composition can be used for treating or preventing bladder cancer and lung cancer.

Any double-stranded molecules of the present invention which target JARID1B gene or any vectors of the present invention which encodes the double-stranded molecule can be used for the present compositions. Details of the double-stranded molecules and the vectors are described above. Preferably, the double-stranded molecule comprises a nucleic acid sequence corresponding to SEQ ID NOs: 21 or 30 as a target sequence. The double-stranded molecules of the present invention are preferably formulated as pharmaceutical compositions prior to administering to a subject, according to techniques known in the art. Pharmaceutical compositions of the present invention are characterized as being at least sterile and pyrogen-free. As used herein, “pharmaceutical compositions” include compositions for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical compositions contain at least one of the double-stranded molecules of the present invention or vectors encoding them (e.g., 0.1 to 90% by weight), or a physiologically acceptable salt of the molecule, mixed with a physiologically acceptable carrier medium. Preferred physiologically acceptable carrier media are water, buffered water, normal saline, 0.4% saline, 0.3% glycine, hyaluronic acid and the like.

According to the present invention, the composition may contain plural kinds of the double-stranded molecules, each of the molecules may be directed to JARID1B gene.

Furthermore, the present composition may contain a vector coding for one or plural double-stranded molecules. For example, the vector may encode one or two kinds of the present double-stranded molecules. Alternatively, the present composition may contain plural kinds of vectors, each of the vectors coding for a different double-stranded molecule.

Moreover, the present double-stranded molecules may be contained in liposomes in the present invention. The details of liposomes are described above.

Pharmaceutical compositions of the present invention can also include conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the present invention can be packaged for use in liquid form, or can be lyophilized.

For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can include any of the carriers and excipients listed above and 10-95%, preferably 25-75%, of one or more double-stranded molecule of the invention. A pharmaceutical composition for aerosol (inhalational) administration can comprise 0.01-20% by weight, preferably 1-10% by weight, of one or more double-stranded molecule of the present invention encapsulated in a liposome as described above, and propellant. A carrier can also be included as desired; e.g., lecithin for intranasal delivery.

In addition to the above, the present composition may contain other pharmaceutical active ingredients so long as they do not inhibit the in vivo function of the present double-stranded molecules. For example, the composition may contain chemotherapeutic agents conventionally used for treating cancers.

In another embodiment, the present invention also provides the use of the double-stranded nucleic acid molecules of the present invention in manufacturing a pharmaceutical composition for treating cancer such as acute myelogenous leukemia, bladder cancer, breast cancer, chronic myelogenous leukemia, cervical cancer, lung cancer, prostate cancer and renal cell carcinoma, especially bladder cancer and lung cancer. For example, the present invention relates to a use of double-stranded nucleic acid molecule inhibiting the expression of JARID1B gene in a cell, which molecule includes a sense strand and an antisense strand complementary thereto. Any of double-stranded molecules of the present invention can be provided for this use so long as the double-stranded molecules have ability to inhibit the expression of JARID1B and cell proliferation when introduced in a cell. The double-stranded molecule comprising the target sequence corresponding to SEQ ID NOs: 21 or 30 is suitable for the use of the present invention.

Alternatively, the present invention further provides a method or process for manufacturing a pharmaceutical composition for treating cancer such as acute myelogenous leukemia, bladder cancer, breast cancer, chronic myelogenous leukemia, cervical cancer, lung cancer, prostate cancer and renal cell carcinoma, wherein the method or process includes a step for formulating a pharmaceutically or physiologically acceptable carrier with the double-stranded molecule of the present invention as active ingredients. The double-stranded molecule comprising the target sequence corresponding to SEQ ID NOs: 21 or 30 is suitable for the method or process of the present invention.

In another embodiment, the present invention also provides a method or process for manufacturing a pharmaceutical composition for treating cancer such as acute myelogenous leukemia, bladder cancer, breast cancer, chronic myelogenous leukemia, cervical cancer, lung cancer, prostate cancer and renal cell carcinoma, wherein the method or process includes a step for admixing an active ingredient with a pharmaceutically or physiologically acceptable carrier, wherein the active ingredient is the double-stranded molecule of the present invention. The double-stranded molecule comprising the target sequence corresponding to SEQ ID NOs: 21 or 30 is suitable for the method or process of the present invention.

In the present invention, a cancer overexpressing JARID1B can be treated with at least one active ingredient selected from the group consisting of:

(a) a double-stranded molecule of the present invention,

(b) DNA encoding thereof, and

(c) a vector encoding thereof.

The cancer includes, but is not limited to, acute myelogenous leukemia, bladder cancer, breast cancer, chronic myelogenous leukemia, cervical cancer, lung cancer, prostate cancer or renal cell carcinoma. Accordingly, prior to the administration of the pharmaceutical composition comprising the active ingredient, it is preferable to confirm whether the expression level of JARID1B in the cancer cells or tissues to be treated is enhanced compared with normal cells of the same organ. Thus, in one embodiment, the present invention provides a method for treating a cancer (over)expressing JARID1B, which method may include the steps of:

i) determining the expression level of JARID1B in cancer cells or tissue(s) obtained from a subject with the cancer to be treated;

ii) comparing the expression level of JARID1B with normal control; and

iii) administrating at least one component selected from the group consisting of

(a) a double-stranded molecule of the present invention,

(b) DNA encoding thereof, and

(c) a vector encoding thereof,

to a subject with a cancer overexpressing JARID 1B compared with normal control.

Alternatively, the present invention also provides a pharmaceutical composition comprising at least one component selected from the group consisting of:

(a) a double-stranded molecule of the present invention,

(b) DNA encoding thereof, and

(c) a vector encoding thereof,

for use in administrating to a subject having a cancer overexpressing JARID1B. In other words, the present invention further provides a method for identifying a subject to be treated with:

(a) a double-stranded molecule of the present invention,

(b) DNA encoding thereof, or

(c) a vector encoding thereof,

, which method may include the step of determining an expression level of JARID1B in subject-derived cancer cells or tissue(s), wherein an increase of the level compared to a normal control level of the gene indicates that the subject has cancer which may be treated with:

(a) a double-stranded molecule of the present invention,

(b) DNA encoding thereof, or

(c) a vector encoding thereof.

The method of treating a cancer of the present invention will be described in more detail below.

A subject to be treated by the present method is preferably a mammal. Exemplary mammals include, but are not limited to, e.g., human, non-human primate, mouse, rat, dog, cat, horse, and cow.

According to the present invention, the expression level of JARID1B in cancer cells or tissues obtained from a subject is determined. The expression level can be determined at the transcription (nucleic acid) product level, using methods known in the art. For example, the mRNA of JARID1B may be quantified using probes by hybridization methods (e.g., Northern hybridization). The detection may be carried out on a chip or an array. The use of an array is preferable for detecting the expression level of JARID1B. Those skilled in the art can prepare such probes utilizing the sequence information of JARID1B. For example, the cDNA of JARID1B may be used as the probes. If necessary, the probes may be labeled with a suitable label, such as dyes, fluorescent substances and isotopes, and the expression level of the gene may be detected as the intensity of the hybridized labels.

Furthermore, the transcription product of JARID1B (e.g., SEQ ID NO: 1) may be quantified using primers by amplification-based detection methods (e.g., RT-PCR). Such primers may be prepared based on the available sequence information of the gene.

Specifically, a probe or primer used for the present method hybridizes under stringent, moderately stringent, or low stringent conditions to the mRNA of JARID1B. As used herein, the phrase “stringent (hybridization) conditions” refers to conditions under which a probe or primer will hybridize to its target sequence, but not to other sequences. Stringent conditions are sequence-dependent and will be different under different circumstances. Specific hybridization of longer sequences is observed at higher temperatures than shorter sequences. Generally, the temperature of a stringent condition is selected to be about 5 degree Centigrade lower than the thermal melting point (Tm) for a specific sequence at a defined ionic strength and pH. The Tm is the temperature (under a defined ionic strength, pH and nucleic acid concentration) at which 50% of the probes complementary to their target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degree Centigrade for short probes or primers (e.g., 10 to 50 nucleotides) and at least about 60 degree Centigrade for longer probes or primers. Stringent conditions may also be achieved with the addition of destabilizing agents, such as formamide.

Alternatively, the translation product may be detected for the diagnosis of the present invention. For example, the quantity of JARID1B protein (SEQ ID NO: 2) may be determined. Methods for determining the quantity of the protein as the translation product include immunoassay methods that use an antibody specifically recognizing the protein. The antibody may be monoclonal or polyclonal. Furthermore, any fragment or modification (e.g., chimeric antibody, scFv, Fab, F(ab′)2, Fv, etc.) of the antibody may be used for the detection, so long as the fragment or modified antibody retains the binding ability to the JARID1B protein. Methods to prepare these kinds of antibodies for the detection of proteins are well known in the art, and any method may be employed in the present invention to prepare such antibodies and equivalents thereof.

As another method to detect the expression level of JARID1B gene based on its translation product, the intensity of staining may be measured via immunohistochemical analysis using an antibody against the JARID1B protein. Namely, in this measurement, strong staining indicates increased presence/level of the protein and, at the same time, high expression level of JARID1B gene.

The expression level of a target gene, e.g., the JARID1B gene, in cancer cells can be determined to be increased if the level increases from the control level (e.g., the level in normal cells) of the target gene by, for example, 10%, 25%, or 50%; or increases to more than 1.1 fold, more than 1.5 fold, more than 2.0 fold, more than 5.0 fold, more than 10.0 fold, or more.

The control level may be determined at the same time with the cancer cells by using a sample(s) previously collected and stored from a subject/subjects whose disease state(s) (cancerous or non-cancerous) is/are known. In addition, normal cells obtained from non-cancerous regions of an organ that has the cancer to be treated may be used as normal control. Alternatively, the control level may be determined by a statistical method based on the results obtained by analyzing previously determined expression level(s) of JARID1B gene in samples from subjects whose disease states are known. Furthermore, the control level can be derived from a database of expression patterns from previously tested cells. Moreover, according to an aspect of the present invention, the expression level of JARID1B gene in a biological sample may be compared to multiple control levels, which are determined from multiple reference samples. It is preferred to use a control level determined from a reference sample derived from a tissue type similar to that of the subject-derived biological sample. Moreover, it is preferred to use the standard value of the expression levels of JARID1B gene in a population with a known disease state. The standard value may be obtained by any method known in the art. For example, a range of mean+/−2 S.D. or mean+/−3 S.D. may be used as the standard value.

In the context of the present invention, a control level determined from a biological sample that is known to be non-cancerous is referred to as a “normal control level”. On the other hand, if the control level is determined from a cancerous biological sample, it is referred to as a “cancerous control level”.

When the expression level of JARID1B gene is increased as compared to the normal control level, or is similar/equivalent to the cancerous control level, the subject may be diagnosed with cancer to be treated.

More specifically, the present invention provides a method of (i) diagnosing whether a subject has the cancer to be treated, and/or (ii) selecting a subject for cancer treatment, which method includes the steps of:

a) determining the expression level of JARID1B in cancer cells or tissue(s) obtained from a subject who is suspected to have the cancer to be treated;

b) comparing the expression level of JARID1B with a normal control level;

c) diagnosing the subject as having the cancer to be treated, if the expression level of JARID1B is increased as compared to the normal control level; and

d) selecting the subject for cancer treatment, if the subject is diagnosed as having the cancer to be treated, in step c).

Alternatively, such a method includes the steps of:

a) determining the expression level of JARID1B in cancer cells or tissue(s) obtained from a subject who is suspected to have the cancer to be treated;

b) comparing the expression level of JARID1B with a cancerous control level;

c) diagnosing the subject as having the cancer to be treated, if the expression level of JARID1B is similar or equivalent to the cancerous control level; and

d) selecting the subject for cancer treatment, if the subject is diagnosed as having the cancer to be treated, in step c).

The present invention also provides a kit for determining a subject suffering from cancer that can be treated with the double-stranded molecule of the present invention or vector encoding thereof, which may also be useful in assessing and/or monitoring the efficacy of a cancer treatment. Preferably, the cancer includes, but is not limited to, acute myelogenous leukemia, bladder cancer, breast cancer, chronic myelogenous leukemia, cervical cancer, lung cancer, prostate cancer or renal cell carcinoma. More particularly, the kit preferably includes at least one reagent for detecting the expression of the JARID1B gene in a subject-derived cancer cell, which reagent may be selected from the group of:

(a) a reagent for detecting mRNA of the JARID1B gene;

(b) a reagent for detecting the JARID1B protein; and

(c) a reagent for detecting the biological activity of the JARID1B protein.

Suitable reagents for detecting mRNA of the JARID1B gene include nucleic acids that specifically bind to or identify the JARID1B mRNA, such as oligonucleotides which have a complementary sequence to a portion of the JARID1B mRNA. These kinds of oligonucleotides are exemplified by primers and probes that are specific to the JARID1B mRNA. These kinds of oligonucleotides may be prepared based on methods well known in the art. If needed, the reagent for detecting the JARID1B mRNA may be immobilized on a solid matrix. Moreover, more than one reagent for detecting the JARID1B mRNA may be included in the kit.

On the other hand, suitable reagents for detecting the JARID1B protein include antibodies to the JARID1B protein. The antibody may be monoclonal or polyclonal. Furthermore, any fragment or modification (e.g., chimeric antibody, scFv, Fab, F(ab′)2, Fv, etc.) of the antibody may be used as the reagent, so long as the fragment or modified antibody retains the binding ability to the JARID1B protein. Methods to prepare these kinds of antibodies for the detection of proteins are well known in the art, and any method may be employed in the present invention to prepare such antibodies and equivalents thereof. Furthermore, the antibody may be labeled with signal generating molecules via direct linkage or an indirect labeling technique. Labels and methods for labeling antibodies and detecting the binding of the antibodies to their targets are well known in the art, and any labels and methods may be employed for the present invention. Moreover, more than one reagent for detecting the JARID1B protein may be included in the kit.

The kit may contain more than one of the aforementioned reagents. For example, tissue samples obtained from subjects without cancer or suffering from cancer, may serve as useful control reagents. A kit of the present invention may further include other materials desirable from a commercial and user standpoint, including buffers, diluents, filters, needles, syringes, and package inserts (e.g., written, tape, CD-ROM, etc.) with instructions for use. These reagents and such may be retained in a container with a label. Suitable containers include bottles, vials, and test tubes. The containers may be formed from a variety of materials, such as glass or plastic.

In an embodiment of the present invention, when the reagent is a probe against the JARID1B mRNA, the reagent may be immobilized on a solid matrix, such as a porous strip, to form at least one detection site. The measurement or detection region of the porous strip may include a plurality of sites, each containing a nucleic acid (probe). A test strip may also contain sites for negative and/or positive controls. Alternatively, control sites may be located on a strip separated from the test strip. Optionally, the different detection sites may contain different amounts of immobilized nucleic acids, i.e., a higher amount in the first detection site and lesser amounts in subsequent sites. Upon the addition of a test sample, the number of sites displaying a detectable signal provides a quantitative indication of the amount of JARID1B mRNA present in the sample. The detection sites may be configured in any suitably detectable shape and are typically in the shape of a bar or dot spanning the width of a test strip.

The kit of the present invention may further include a positive control sample or JARID1B standard sample. The positive control sample of the present invention may be prepared by collecting JARID1B positive samples and then assaying their JARID1B levels. Alternatively, a purified JARID1B protein or polynucleotide may be added to cells that do not express JARID1B to form the positive sample or the JARID1B standard sample. In the present invention, purified JARID1B may be a recombinant protein. The JARID1B level of the positive control sample is, for example, more than the cut off value.

Screening for an Anti-Cancer Compound:

The present invention provides the method of screening for a compound which has ability to inhibit the growth of cancer cells, for example, acute myelogenous leukemia, bladder cancer, breast cancer, chronic myelogenous leukemia, cervical cancer, lung cancer, prostate cancer and renal cell carcinoma. The present method of screening can be performed by targeting JARID1B gene or protein. The compounds screened by the present method may be good candidate compounds for treating cancer such as acute myelogenous leukemia, bladder cancer, breast cancer, chronic myelogenous leukemia, cervical cancer, lung cancer, prostate cancer and renal cell carcinoma. Especially, it was confirmed that inhibiting JARID1B gene expression led to suppress cell proliferation in bladder cancer and lung cancer (FIG. 7). Therefore, the compounds screened by the present method may be preferably used for treating bladder cancer and lung cancer.

Agents or Compounds for Screening:

In the context of the present invention, agents or compounds to be identified through the present screening methods may be any compounds or compositions including several compounds. Furthermore, a test agent or composition exposed to a cell or protein according to the screening methods of the present invention may be a single compound or a combination of compounds. When a combination of compounds is used in the methods, the compounds may be contacted sequentially or simultaneously.

Any test agents or compounds, for example, cell extracts, cell culture supernatant, products of fermenting microorganism, extracts from marine organism, plant extracts, purified or crude proteins, peptides, non-peptide compounds, synthetic micromolecular compounds (including nucleic acid constructs, such as antisense RNA, siRNA, Ribozymes, and aptamer etc or antibody.) and natural compounds can be used in the screening methods of the present invention. The test agent or compound of the present invention can be also obtained using any of the numerous approaches in combinatorial library methods known in the art, including (1) biological libraries, (2) spatially addressable parallel solid phase or solution phase libraries, (3) synthetic library methods requiring deconvolution, (4) the “one-bead one-compound” library method and (5) synthetic library methods using affinity chromatography selection. The biological library methods using affinity chromatography selection is limited to peptide libraries, while the other four approaches are applicable to peptide, non-peptide oligomer or small molecule libraries of compounds (Lam, Anticancer Drug Des 1997, 12: 145-67). Examples of methods for the synthesis of molecular libraries can be found in the art (DeWitt et al., Proc Natl Acad Sci USA 1993, 90: 6909-13; Erb et al., Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Carell et al., Angew Chem Int Ed Engl 1994, 33: 2061; Gallop et al., J Med Chem 1994, 37: 1233-51). Libraries of compounds may be presented in solution (see Houghten, Bio/Techniques 1992, 13: 412-21) or on beads (Lam, Nature 1991, 354: 82-4), chips (Fodor, Nature 1993, 364: 555-6), bacteria (U.S. Pat. No. 5,223,409), spores (U.S. Pat. No. 5,571,698; 5,403,484, and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6; Cwirla et al., Proc Natl Acad Sci USA 1990, 87: 6378-82; Felici, J Mol Biol 1991, 222: 301-10; US Pat. Application 2002103360).

A compound in which a part of the structure of the compound screened by any of the present screening methods is converted by addition, deletion and/or replacement, is included in the agents or compounds obtained by the screening methods of the present invention.

Furthermore, when the screened test agent or compound is a protein, for obtaining a DNA encoding the protein, either the whole amino acid sequence of the protein may be determined to deduce the nucleic acid sequence coding for the protein, or partial amino acid sequence of the obtained protein may be analyzed to prepare an oligo DNA as a probe based on the sequence, and screen cDNA libraries with the probe to obtain a DNA encoding the protein. The obtained DNA is confirmed it's usefulness in preparing the test agent which is a candidate for treating or preventing cancer.

Test agents or compound useful in the screenings described herein can also be antibodies that specifically bind to JARID1B protein or partial peptides thereof that lack the biological activity of the original proteins in vivo.

Although the construction of test agent or compound libraries is well known in the art, herein below, additional guidance in identifying test agents and construction libraries of such agents or compounds for the present screening methods are provided.

(i) Molecular Modeling:

Construction of test agent or compound libraries is facilitated by knowledge of the molecular structure of compounds known to have the properties sought, and/or the molecular structure of the target molecules to be inhibited, i.e., JARID1B protein. One approach to preliminary screening of test agents or compounds suitable for further evaluation is computer modeling of the interaction between the test agent and JARID1B protein.

Computer modeling technology allows the visualization of the three-dimensional atomic structure of a selected molecule and the rational design of new compounds that will interact with the molecule. The three-dimensional construct typically depends on data from x-ray crystallographic analysis or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

An example of the molecular modeling system described generally above includes the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen et al. Acta Pharmaceutica Fennica 1988, 97: 159-66; Ripka, New Scientist 1988, 54-8; McKinlay & Rossmann, Annu Rev Pharmacol Toxiciol 1989, 29: 111-22; Perry & Davies, Prog Clin Biol Res 1989, 291: 189-93; Lewis & Dean, Proc R Soc Lond 1989, 236: 125-40, 141-62; and, with respect to a model receptor for nucleic acid components, Askew et al., J Am Chem Soc 1989, 111: 1082-90.

Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. See, e.g., DesJarlais et al., J Med Chem 1988, 31: 722-9; Meng et al., J Computer Chem 1992, 13: 505-24; Meng et al., Proteins 1993, 17: 266-78; Shoichet et al., Science 1993, 259: 1445-50. Once a putative inhibitor has been identified, combinatorial chemistry techniques can be employed to construct any number of variants based on the chemical structure of the identified putative inhibitor, as detailed below. The resulting library of putative inhibitors, or “test agents” may be screened using the methods of the present invention to identify test agents or compounds treating or preventing cancers.

(ii) Combinatorial Chemical Synthesis:

Combinatorial libraries of test agents or compounds may be produced as part of a rational drug design program involving knowledge of core structures existing in known inhibitors. This approach allows the library to be maintained at a reasonable size, facilitating high throughput screening. Alternatively, simple, particularly short, polymeric molecular libraries may be constructed by simply synthesizing all permutations of the molecular family making up the library. An example of this latter approach would be a library of all peptides six amino acids in length. Such a peptide library could include every 6 amino acid sequence permutation. This type of library is termed a linear combinatorial chemical library.

Preparation of combinatorial chemical libraries is well known to those of skill in the art, and may be generated by either chemical or biological synthesis. Combinatorial chemical libraries include, but are not limited to, peptide libraries (see, e.g., U.S. Pat. No. 5,010,175; Furka, Int J Pept Prot Res 1991, 37: 487-93; Houghten et al., Nature 1991, 354: 84-6). Other chemistries for generating chemical diversity libraries can also be used. Such chemistries include, but are not limited to: peptides (e.g., PCT Publication No. WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiazepines and dipeptides (DeWitt et al., Proc Natl Acad Sci USA 1993, 90:6909-13), vinylogous polypeptides (Hagihara et al., J Amer Chem Soc 1992, 114: 6568), nonpeptidal peptidomimetics with glucose scaffolding (Hirschmann et al., J Amer Chem Soc 1992, 114: 9217-8), analogous organic syntheses of small compound libraries (Chen et al., J. Amer Chem Soc 1994, 116: 2661), oligocarbamates (Cho et al., Science 1993, 261: 1303), and/or peptidylphosphonates (Campbell et al., J Org Chem 1994, 59: 658), nucleic acid libraries (see Ausubel, Current Protocols in Molecular Biology 1995 supplement; Sambrook et al., Molecular Cloning: A Laboratory Manual, 1989, Cold Spring Harbor Laboratory, New York, USA), peptide nucleic acid libraries (see, e.g., U.S. Pat. No. 5,539,083), antibody libraries (see, e.g., Vaughan et al., Nature Biotechnology 1996, 14(3):309-14 and PCT/US96/10287), carbohydrate libraries (see, e.g., Liang et al., Science 1996, 274: 1520-22; U.S. Pat. No. 5,593,853), and small organic molecule libraries (see, e.g., benzodiazepines, Gordon EM. Curr Opin Biotechnol. 1995 Dec. 1;6(6):624-31.; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

(iii) Phage Display:

Another approach uses recombinant bacteriophage to produce libraries. Using the “phage method” (Scott & Smith, Science 1990, 249: 386-90; Cwirla et al., Proc Natl Acad Sci USA 1990, 87: 6378-82; Devlin et al., Science 1990, 249: 404-6), very large libraries can be constructed (e.g., 106 -108 chemical entities). A second approach uses primarily chemical methods, of which the Geysen method (Geysen et al., Molecular Immunology 1986, 23: 709-15; Geysen et al., J Immunologic Method 1987, 102: 259-74); and the method of Fodor et al. (Science 1991, 251: 767-73) are examples. Furka et al. (14th International Congress of Biochemistry 1988, Volume #5, Abstract FR:013; Furka, Int J Peptide Protein Res 1991, 37: 487-93), Houghten (U.S. Pat. No. 4,631,211) and Rutter et al. (U.S. Pat. No. 5,010,175) describe methods to produce a mixture of peptides that can be tested as agonists or antagonists.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Screening for a JARID1B Binding Agent or Compound:

In the present invention, over-expression of JARID1B was detected in acute myelogenous leukemia, bladder cancer, breast cancer, chronic myelogenous leukemia, cervical cancer, lung cancer, prostate cancer and renal cell carcinoma, comparing the expression in normal organs (Table 3). Therefore, the present invention provides a method of screening for an agent that binds to JARID1B protein. Due to the expression of JARID1B gene in such cancer, an agent or compound binds to JARID1B protein is expected to suppress the proliferation of cancer cells, and thus be useful for treating or preventing cancer. Therefore, the present invention also provides a method for screening an agent or compound that suppresses the proliferation of cancer cells, and a method for screening a candidate agent or compound for treating or preventing lung cancer using JARID1B polypeptide. Specially, an embodiment of this screening method includes the steps of:

(a) contacting a test agent or compound with a JARID1B polypeptide derived from JARID1B gene;

(b) detecting the binding activity between the polypeptide and the test agent or compound; and

(c) selecting the test agent or compound that bind to the polypeptide as a candidate agent or compound.

According to the present invention, the therapeutic effect of the test agent or compound on inhibiting the cell growth or a candidate agent or compound for treating or preventing JARID1B associating disease may be evaluated. Therefore, the present invention also provides a method for screening a candidate agent or compound that suppresses the proliferation of cancer cells, and a method for screening a candidate agent or compound for treating or preventing cancer.

More specifically, the method includes the steps of:

(a) a test agent or compound with a JARID1B polypeptide derived from JARID1B gene;

(b) detecting the binding level between the polypeptide and the test or compound;

(c) comparing the binding level of (b) with that detected in the absence of the test agent or compound; and

(d) correlating the binding level of (c) with the therapeutic effect of the test agent or compound.

In the present invention, the therapeutic effect may be correlated with the binding level of the JARID1B polypeptide. For example, when the test agent or compound binds to the JARID1B polypeptide, the test agent or compound may identified or selected as the candidate agent or compound having the therapeutic effect. Alternatively, when the test agent or compound does not bind to the JARID1B polypeptide, the test agent or compound may identified as the agent or compound having no significant therapeutic effect.

The method of the present invention will be described in more detail below.

JARID1B polypeptide to be used for the present screening method may be a recombinant polypeptide or a protein derived from the nature or a partial peptide thereof. The polypeptide to be contacted with a test agent or compound can be, for example, a purified polypeptide, a soluble protein, a form bound to a carrier or a fusion protein fused with other polypeptides.

As a method of screening for proteins, for example, that bind to JARID1B polypeptide, many methods well known by a person skilled in the art can be used. Such a screening can be conducted by, for example, immunoprecipitation method, specifically, in the following manner. The gene encoding JARID1B polypeptide is expressed in host (e.g., animal) cells and so on by inserting the gene to an expression vector for foreign genes, such as pSV2neo, pcDNA I, pcDNA3.1, pCAGGS and pCD8.

The promoter to be used for the expression may be any promoter that can be used commonly and include, for example, the SV40 early promoter (Rigby in Williamson (ed.), Genetic Engineering, vol. 3. Academic Press, London, 83-141 (1982)), the EF-alpha promoter (Kim et al., Gene 91: 217-23 (1990)), the CAG promoter (Niwa et al., Gene 108: 193 (1991)), the RSV LTR promoter (Cullen, Methods in Enzymology 152: 684-704 (1987)) the SR alpha promoter (Takebe et al., Mol Cell Biol 8: 466 (1988)), the CMV immediate early promoter (Seed and Aruffo, Proc Natl Acad Sci USA 84: 3365-9 (1987)), the SV40 late promoter (Gheysen and Fiers, J Mol Appl Genet 1: 385-94 (1982)), the Adenovirus late promoter (Kaufman et al., Mol Cell Biol 9: 946 (1989)), the HSV TK promoter and so on.

The introduction of the gene into host cells to express a foreign gene can be performed according to any methods, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1641-3 (1984)), the Lipofectin method (Derijard B., Cell 76: 1025-37 (1994); Lamb et al., Nature Genetics 5: 22-30 (1993): Rabindran et al., Science 259: 230-4 (1993)) and so on.

The polypeptide encoded by JARID1B gene can be expressed as a fusion protein including a recognition site (epitope) of a monoclonal antibody by introducing the epitope of the monoclonal antibody, whose specificity has been revealed, to the N- or C-terminus of the polypeptide. A commercially available epitope-antibody system can be used (Experimental Medicine 13: 85-90 (1995)). Vectors which can express a fusion protein with, for example, beta-galactosidase, maltose binding protein, glutathione S-transferase, green florescence protein (GFP) and so on by the use of its multiple cloning sites are commercially available. Also, a fusion protein prepared by introducing only small epitopes consisting of several to a dozen amino acids so as not to change the property of JARID1B polypeptide by the fusion is also reported. Epitopes, such as polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such, and monoclonal antibodies recognizing them can be used as the epitope-antibody system for screening proteins binding to JARID1B polypeptide (Experimental Medicine 13: 85-90 (1995)).

In immunoprecipitation, an immune complex is formed by adding these antibodies to cell lysate prepared using an appropriate detergent. The immune complex consists of JARID1B polypeptide, a polypeptide including the binding ability with the polypeptide, and an antibody. Immunoprecipitation can be also conducted using antibodies against JARID1B polypeptide, besides using antibodies against the above epitopes. An immune complex can be precipitated, for example by Protein A sepharose or Protein G sepharose when the antibody is a mouse IgG antibody. If the polypeptide encoded by JARID1B gene is prepared as a fusion protein with an epitope, such as GST, an immune complex can be formed in the same manner as in the use of the antibody against JARID1B polypeptide, using a substance specifically binding to these epitopes, such as glutathione-Sepharose 4B.

Immunoprecipitation can be performed by following or according to, for example, the methods in the literature (Harlow and Lane, Antibodies, 511-52, Cold Spring Harbor Laboratory publications, New York (1988)).

SDS-PAGE is commonly used for analysis of immunoprecipitated proteins and the bound protein can be analyzed by the molecular weight of the protein using gels with an appropriate concentration. Since the protein bound to the JARID1B polypeptide is difficult to detect by a common staining method, such as Coomassie staining or silver staining, the detection sensitivity for the protein can be improved by culturing cells in culture medium containing radioactive isotope, ³⁵S-methionine or ³⁵S-cystein, labeling proteins in the cells, and detecting the proteins. The target protein can be purified directly from the SDS-polyacrylamide gel and its sequence can be determined, when the molecular weight of a protein has been revealed.

As a method of screening for proteins binding to JARID1B polypeptide using the polypeptide, for example, West-Western blotting analysis (Skolnik et al., Cell 65: 83-90 (1991)) can be used. Specifically, a protein binding to JARID1B polypeptide can be obtained by preparing a cDNA library from cultured cells expected to express a protein binding to JARID1B polypeptide using a phage vector (e.g., ZAP), expressing the protein on LB-agarose, fixing the protein expressed on a filter, reacting the purified and labeled JARID1B polypeptide with the above filter, and detecting the plaques expressing proteins bound to JARID1B polypeptide according to the label. The polypeptide of the invention may be labeled by utilizing the binding between biotin and avidin, or by utilizing an antibody that specifically binds to JARID1B polypeptide, or a peptide or polypeptide (for example, GST) that is fused to JARID1B polypeptide. Methods using radioisotope or fluorescence and such may be also used.

Alternatively, in another embodiment of the screening method of the present invention, a two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet 10: 286-92 (1994)”).

In the two-hybrid system, JARID1B polypeptide is fused to the SRF-binding region or GAL4-binding region and expressed in yeast cells. A cDNA library is prepared from cells expected to express a protein binding to JARID1B polypeptide, such that the library, when expressed, is fused to the VP16 or GAL4 transcriptional activation region. The cDNA library is then introduced into the above yeast cells and the cDNA derived from the library is isolated from the positive clones detected (when a protein binding to JARID1B polypeptide is expressed in yeast cells, the binding of the two activates a reporter gene, making positive clones detectable). A protein encoded by the cDNA can be prepared by introducing the cDNA isolated above to E. coli and expressing the protein. As a reporter gene, for example, Ade2 gene, lacZ gene, CAT gene, luciferase gene and such can be used in addition to the HIS3 gene.

An agent or compound binding to the polypeptide encoded by JARID1B gene can also be screened using affinity chromatography. For example, JARID1B polypeptide may be immobilized on a carrier of an affinity column, and a test agent or compound, containing a protein capable of binding to JARID1B polypeptide, is applied to the column. A test agent or compound herein may be, for example, cell extracts, cell lysates, etc. After loading the test agent, the column is washed, and agents bound to JARID1B polypeptide can be prepared. When the test agent or compound is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon may be used as a mean for detecting or quantifying the bound agent in the present invention. When such a biosensor is used, the interaction between JARID1B polypeptide and a test agent can be observed real-time as a surface plasmon resonance signal, using only a minute amount of polypeptide and without labeling (for example, BIAcore, Pharmacia). Therefore, it is possible to evaluate the binding between JARID1B polypeptide and a test agent or compound using a biosensor such as BIAcore.

The methods of screening for molecules that bind when the immobilized JARID1B polypeptide is exposed to synthetic chemical compounds, or natural substance banks or a random phage peptide display library, and the methods of screening using high-throughput based on combinatorial chemistry techniques (Wrighton et al., Science 273: 458-64 (1996); Verdine, Nature 384: 11-13 (1996); Hogan, Nature 384: 17-9 (1996)) to isolate not only proteins but chemical compounds that bind to the JARID1B protein (including agonist and antagonist) are well known to one skilled in the art.

In the present invention, it is revealed that suppressing the expression of JARID1B, reduces cell growth. Thus, by screening for candidate compounds that binds to the JARID1B polypeptide, candidate compounds that have the potential to treat or prevent cancers can be identified. Potential of these candidate compounds to treat or prevent cancers may be evaluated by second and/or further screening to identify therapeutic agent or compound for cancers.

Screening for an Agent or Compound Suppressing the Biological Activity of JARID1B:

JARID1B protein has the histone H3 demethylation activity (Yamane K. et al. Mol Cell 2007;25: 801-12), and according to the present invention, the protein has the activity of promoting the expression of E2F1 gene and E2F2 gene (FIG. 9), promoting cell proliferation activity (FIG. 7C) or anti-apoptosis activity (FIG. 7E). Using these biological activities, the present invention provides a method for screening an agent or compound that suppresses the proliferation of cancer cells which is relating to the overexpression of JARID1B gene, and a method for screening a candidate agent or compound for treating or preventing such cancer. Thus, the present invention provides a method including the steps as follows:

(a) contacting a test agent or compound with a polypeptide derived from JARID1B gene;

(b) detecting the biological activity of the polypeptide of step (a); and

(c) selecting the test agent or compound that suppresses the biological activity as compared to the biological activity in the absence of the test agent or compound as a candidate agent or compound.

According to the present invention, the therapeutic effect of the test agent or compound on inhibiting the cell growth or a candidate agent or compound for treating or preventing JARID1B associating cancer may be evaluated. Therefore, the present invention also provides a method of screening for a candidate agent or compound for inhibiting the cell growth or a candidate agent or compound for treating or preventing JARID1B associating cancer, using the JARID1B polypeptide or fragments thereof including the steps as follows:

a) contacting a test agent or compound with the JARID1B polypeptide or a functional fragment thereof; and

b) detecting the biological activity of the polypeptide or fragment of step (a), and

c) correlating the biological activity of b) with the therapeutic effect of the test agent or compound.

In the present invention, the therapeutic effect may be correlated with the biological activity of JARID1B polypeptide or a functional fragment thereof. For example, when the test agent or compound suppresses or inhibits the biological activity of JARID1B polypeptide or a functional fragment thereof as compared to a level detected in the absence of the test agent or compound, the test agent or compound may identified or selected as the candidate agent or compound having the therapeutic effect. Alternatively, when the test agent or compound does not suppress or inhibit the biological activity JARID1B polypeptide or a functional fragment thereof as compared to a level detected in the absence of the test agent or compound, the test agent or compound may be identified as the agent or compound having no significant therapeutic effect. The method of the present invention will be described in more detail below. Any polypeptides can be used for screening so long as they include the biological activity of JARID1B protein. Such biological activity includes histone H3 demethylation activity, the activity of promoting the E2F1 and the E2F2 gene expression, cell proliferation activity and anti-apoptosis activity. For example, JARID1B protein can be used and polypeptides functionally equivalent to these proteins can also be used. Such polypeptides may be expressed endogenously or exogenously by cells.

In another aspect, the present invention also provides a screening method following the method described in “Screening for a JARID1B binding agent or compound”, comprising the steps of:

a) contacting a test agent or compound with JARID1B polypeptide derived from JARID1B gene;

b) detecting the binding between the polypeptide and the test agent or compound;

c) selecting the test agent or compound that binds to the polypeptide;

d) contacting the test agent or compound selected in step c) with JARID1B polypeptide;

e) comparing the biological activity of the polypeptide with the biological activity detected in the absence of the test agent or compound; and

f) selecting the test agent or compound that suppresses the biological activity of the polypeptide as a candidate agent or compound for treating or preventing cancer.

The agents or compounds isolated by this screening are candidates for antagonists of the polypeptide encoded by JARID1B gene. The term “antagonist” refers to molecules that inhibit the function of the polypeptide by binding thereto. Said term also refers to molecules that reduce or inhibit the expression of JARID1B gene. Moreover, an agent or compound isolated by this screening is a candidate for agents or compounds which inhibit the in vivo interaction of JARID1B polypeptide with molecules (including DNAs and proteins).

When the biological activity to be detected in the present method is cell proliferation, it can be detected, for example, by preparing cells which express the JARID1B polypeptide, culturing the cells in the presence of a test compound, and determining the speed of cell proliferation, measuring the cell cycle and such, as well as by measuring survival cells or the colony forming activity. The compounds that reduce the speed of proliferation of the cells expressed JARID1B are selected as candidate compound for treating or preventing cancer.

More specifically, the method includes the step of:

(a) contacting a test compound with cells overexpressing JARID1B;

(b) measuring cell-proliferating activity; and

(c) selecting the test compound that reduces the cell-proliferating activity in the comparison with the cell-proliferating activity in the absence of the test compound. In preferable embodiments, the method of the present invention may further include the steps of:

(d) selecting the test compound that have no effect to the cells no or little expressing JARID1B.

When the biological activity to be detected in the present method is anti-apoptosis, it can be determined by usual methods performed by those skilled in the art such as measuring the number of sub-G1 cells, TUNEL method or LM-PCR method using various commercially available kits. For example, the number of sub-G1 cells can be determined by using FACS. Apoptosis can be also examined by TUNEL method using Apotag Direct (oncor) or LM-PCR using an ApoAlert LM-PCR ladder assay kit (Clontech) according to the attached manual.

When the biological activity to be detected in the present method is the demethylation activity, it can be determined by contacting a JARID1B polypeptide with a substrate (e.g., the histone H3 comprising tri- or di-methylated lysine 4) under a suitable condition for demethylation of the substrate and detecting the demethylation level of the substrate.

More specifically, the method includes the steps of:

(a) contacting a JARID1B polypeptide with a substrate to be demethylated in the presence of the test agent or compound under the condition capable of demethylation of substrate.

(b) detecting the methylation level of the substrate; and

(c) selecting the test agent or compound that increases the methylation level of the substrate as compared to the methylation level detected in the absence of the test agent as the candidate agent.

Preferably, a substrate to be demethlated by a JARID1B polypeptide is a histone H3 or the fragment thereof comprising tri-or di-methylated lysine 4 of histone H3.

In the present invention, the demethylation activity of a JARID1B polypeptide can be determined by methods known in the art. For example, a JARID1B polypeptide can be incubated with a substrate with a labeled methylation site, under a suitable condition for demethylation. For example, a histone H3 peptide having tri- or di-[methyl-¹⁴C]-lysine , or tri- or di-[methyl-³H]-lysine in 4^{th} amino acid residue can be preferably used as a substrate for demethylation. The demethylation activity can be determined based on the radioactivity in the substrate after incubation (i.e., the higher radioactivity in the substrate indicates the lower demethylation activity of a JARID1B polypeptide). The radioactivity in the substrate may be detected, for example, by SDS-polyacrylamide gel electrophoresis and autoradiography. Alternatively, following the incubation the substrate may be separated from the JARID1B by conventional methods such as gel filtration and immunoprecipitation, and the radioactivity in the substrate may be measured by methods well-known in the art. Other suitable labels that can be attached to methyl group in a substrate, such as chromogenic and fluorescent labels, and methods of detecting these labels, are known in the art.

Alternatively, demethylation activity of a JARID1B polypeptide may be determined using a mass spectrometry or reagents that selectively recognize a methylated substrate. For example, antibodies against the methylated substrate may be preferably used as such reagents. Any immunological techniques using such antibodies can be used for the detection of methylation level of the substrate. For example, when the substrate is a methylated histone, antibodies against a methylated histone (e.g., a histone H3 comprising tri- or di-methylated lysine 4) may be preferably used. Such antibodies are commercially available (e.g., Abcam Ltd.). For example, ELISA or Immunoblotting with antibodies recognizing a methylated substrate may be used for the present invention.

Furthermore, the present method detecting demethylation activity can be performed by preparing cells which express the JARID1B gene, culturing the cells in the presence of a test compound, and determining the methylation level of histones in the cells, for example, by using the antibody specific binding to the methylation region of the histone.

More specifically, the method includes the step of:

(a) contacting a test agent or compound with a cell expressing JARID1B gene;

(b) detecting the methylation level of the histone H3 lysine 4; and

(c) selecting the test agent or compound that increases the methylation level as compared to the methylation level detected in the absence of the test agent or compound as a candidate agent or compound.

Alternatively, when the biological activity to be detected is the activity of promoting E2F1 or E2F2 gene expression, it can be detected, for example, by E2F reporter assay shown in Example 1. For this method, a test compound is contacted with cells expressing JARID1B gene, such as cancer cells.

More specifically, the method includes the steps of:

(a) contacting a test agent or compound with cells expressing JARID1B gene;

(b) measuring the expression level of E2F1 gene or E2F2 gene; and

(c) selecting the test agent or compound that reduces the expression level of E2F1 gene or E2F2 gene as compared to the expression level in the absence of the test agent or compound as a candidate agent or compound.

The exemplary nucleic acid and polypeptide sequences of E2F1 gene are shown in SEQ ID NO: 35 and 36 respectively, but not limited to those. Furthermore, the sequence data are also available via accession number of NM_{—}005225.2 and NP_{—}005216.1 respectively, for example.

The exemplary nucleic acid and polypeptide sequences of E2F2 gene are also shown in SEQ ID NO: 37 and 38 respectively, but not limited to those. Furthermore, the sequence data are also available via accession number of NM_{—}004091.2 and NP_{—}004082.1 respectively, for example.

Cells expressing JARID1B gene include, for example, cell lines established from cancer (e.g., 5637, 253J, 253J-BV, HT1197, HT1376, J82, SCaBER and UMUC3 for bladder cancer, SBC5 or H2170 and LC319 for lung cancer) and purified cells from clinical cancer tissues, such cells can be used for the present screening method. Measurement of the expression level of E2F1 gene or E2F2 gene can be carried out by introducing a vector comprising the transcriptional regulatory region of E2F1 gene or E2F2 gene and a reporter gene under control of the regulatory region into the cell before or after contacted with the test agent or compound, and then detecting the expression level of the reporter gene. The transcriptional regulatory region of E2F1 gene and E2F2 gene is well known in the art (e.g. Araki et al. Oncogene 2003, 22: 7632-41, Pilon et al. Mol Cell Biol 2008, 28: 7394-401). Reporter genes can be, for example, luciferase, green florescence protein (GFP), Discosoma sp. Red Fluorescent Protein (DsRed), Chrolamphenicol Acetyltransferase (CAT), lacZ and beta-glucuronidase (GUS) and so on. Besides, some commercial products are available, such as the Cignal™ E2F Reporter Assay Kit (SuperArray Bioscience Corporation) for E2F reporter assay. Alternatively, the expression level of E2F1 gene and E2F2 gene may be determined by detecting the transcription or translation product of these gene directly using methods well-known in the art, such as RT-PCR, Northern blot assay, Western blot assay, immunostaining, ELISA assay and flow cytometry analysis.

In the present invention, methods for preparing polypeptides functionally equivalent to a given protein are well-known by a person skilled in the art and include known methods of introducing mutations into the protein. Generally, it is known that modifications of one or more amino acid in a protein do not influence the function of the protein (Mark D F et al., Proc Natl Acad Sci USA 1984, 81: 5662-6; Zoller M J & Smith M, Nucleic Acids Res 1982, 10: 6487-500; Wang A et al., Science 1984, 224:1431-3; Dalbadie-McFarland Get al., Proc Natl Acad Sci USA 1982, 79: 6409-13). In fact, mutated or modified proteins, proteins having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that individual additions, deletions, insertions, or substitutions to an amino acid sequence which alter a single amino acid or a small percentage of amino acids, or those considered to be “conservative modifications”, wherein the alteration of a protein results in a protein with similar functions, are contemplated in the context of the instant invention.

“Suppress the biological activity” as defined herein are preferably at least 10% suppression of the biological activity of JARID1B in comparison with in absence of the agent or compound, more preferably at least 25%, 50% or 75% suppression and most preferably at 90% suppression.

In the present invention, it is revealed that suppressing the expression of JARID1B, reduces cell growth. Thus, by screening for candidate compounds that inhibits the biological activity of the JARID1B polypeptide, candidate compounds that have the potential to treat or prevent cancers can be identified. Potential of these candidate compounds to treat or prevent cancers may be evaluated by second and/or further screening to identify therapeutic agent or compound for cancers. For example, when a compound binding to JARID1B protein inhibits described above activities of the cancer, it may be concluded that such compound has the JARID1B specific therapeutic effect.

In the preferred embodiments, control cells which do not express JARID1B polypeptide are used. Accordingly, the present invention also provides a method of screening for a candidate substance for inhibiting the cell growth or a candidate substance for treating or preventing JARID1B associating cancer, using the JARID1B polypeptide or fragments thereof including the steps as follows:

a) culturing cells which express a JARID1B polypeptide or a functional fragment thereof, and control cells that do not express a JARID1B polypeptide or a functional fragment thereof in the presence of the test substance;

b) detecting the biological activity of the cells which express the protein and control cells; and

c) selecting the test compound that inhibits the biological activity in the cells which express the protein as compared to the proliferation detected in the control cells and in the absence of said test substance.

Screening for an Agent or Compound Altering the Expression of JARID1B:

In the present invention, the decrease of the expression of JARID1B gene by siRNA causes inhibiting cancer cell proliferation (FIG. 7). Therefore, the present invention provides a method of screening for an agent or compound that inhibits the expression of JARID1B gene. An agent or compound that inhibits the expression of JARID1B gene is expected to suppress the proliferation of cancer cells, and thus is useful for treating or preventing cancer. Therefore, the present invention also provides a method for screening an agent or compound that suppresses the proliferation of cancer cells, and a method for screening an agent or compound for treating or preventing cancer. According to the present invention, the expression of JARID1B gene was confirmed to be elevated in acute myelogenous leukemia, bladder cancer, breast cancer, chronic myelogenous leukemia, cervical cancer, lung cancer, prostate cancer and renal cell carcinoma. Thus, agents or compounds screened by the present method may be suitable to treat these cancers. More specifically, bladder cancer and lung cancer are suitable targets for the agents or compounds screened by the present method since a compound inhibiting the expression of JARID1B gene was confirmed to suppress cell proliferation in those cancers (FIG. 7).

In the context of the present invention, such screening may include, for example, the following steps:

(a) contacting a test agent or compound with a cell expressing JARID1B gene;

(b) detecting the expression level of JARID1B gene; and

(c) selecting the test agent or compound that reduces the expression level of JARID1B gene as compared to the expression level in the absence of the test agent or compound as a candidate agent or compound.

According to the present invention, the therapeutic effect of the test agent or compound on inhibiting the cell growth or a candidate agent or compound for treating or preventing JARID1B associating cancer may be evaluated. Therefore, the present invention also provides a method for screening a candidate agent or compound that suppresses the proliferation of cancer cells, and a method for screening a candidate agent or compound for treating or preventing JARID1B associating cancer.

In the context of the present invention, such screening may include, for example, the following steps:

a) contacting a test agent or compound with a cell expressing the JARID1B gene;

b) detecting the expression level of the JARID1B gene; and

c) correlating the expression level of b) with the therapeutic effect of the test agent or compound.

In the present invention, the therapeutic effect may be correlated with the expression level of the JARID1B gene. For example, when the test agent or compound reduces the expression level of the JARID1B gene as compared to a level detected in the absence of the test agent or compound, the test agent or compound may identified or selected as the candidate agent or compound having the therapeutic effect. Alternatively, when the test agent or compound does not reduce the expression level of the JARID1B gene as compared to a level detected in the absence of the test agent or compound, the test agent or compound may be identified as the agent or compound having no significant therapeutic effect.

The method of the present invention will be described in more detail below.

Cells expressing JARID1B gene include, for example, cell lines established from cancer (e.g., 5637, 253J, 253J-BV, HT1197, HT1376, J82, SCaBER and UMUC3 for bladder cancer, SBC5 or H2170 and LC319 for lung cancer) and purified cells from clinical cancer tissues, such cells can be used for the above screening of the present invention. The expression level can be estimated by methods well-known to one skilled in the art, for example, RT-PCR, Northern blot assay, Western blot assay, immunostaining or flow cytometry analysis. “Reduce the expression level” as defined herein are preferably at least 10% reduction of expression level of JARID1B gene in comparison to the expression level in absence of the agent or compound, more preferably at least 25%, 50% or 75% reduced level and most preferably at 95% reduced level. The agent or compound herein includes chemical compounds, double-stranded molecules, and so on. The preparation of double-stranded molecules is in the aforementioned description. In the method of screening, a test agent or compound that reduces the expression level of JARID1B gene can be selected as an agent or compound to be used for inhibit cancer cell proliferation, and thus, expected to be a candidate agent or compound for the treatment or prevention of cancer.

In the present invention, it is revealed that suppressing the expression of JARID1B, reduces cell growth. Thus, by screening for candidate compounds that inhibits the biological activity of the JARID1B polypeptide, candidate compounds that have the potential to treat or prevent cancers can be identified. Potential of these candidate compounds to treat or prevent cancers may be evaluated by second and/or further screening to identify therapeutic agent or compound for cancers.

Furthermore, the expression level of JARID1B gene can be detected as the expression level of E2F1 gene or E2F2 gene as the expression of these gene were confirmed to be suppressed by inhibition of JARID1B gene expression in the present invention. Measurement of E2F1 gene and E2F2 gene expression level can be determined by the aforementioned E2F reporter assay or detecting transcription or translation product of E2F1 gene or E2F2 gene.

Specifically, the present screening method may include the following steps:

(a) contacting a test agent or compound with cells expressing JARID1B gene;

(b) measuring the expression level of the E2F1 or E2F2 gene; and

(c) selecting the test agent or compound that reduces the expression level of E2F1 gene or E2F2 gene as compared to the expression level in the absence of the test agent or compound as a candidate agent or compound.

In preferable embodiments, the method of the present invention may further include the steps of:

(d) selecting the test agent or compound that have no effect against the control cells which hardly express JARID1B.

According to the present invention, it is revealed that suppressing the expression of JARID1B, reduces cell growth. Further, it is revealed that the expression of E2F1 gene and E2F2 gene were suppressed by inhibition of JARID1B gene expression. Therefore, the present invention also provides a method for screening a candidate agent or compound that suppresses the proliferation of cancer cells, and a method for screening a candidate agent or compound for treating or preventing JARID1B associating cancer.

In the context of the present invention, such screening may include, for example, the following steps:

a) contacting a test agent or compound with a cell expressing the JARID1B gene;

b) detecting the expression level of the E2F1 or E2F2 gene; and

c) correlating the expression level of b) with the therapeutic effect of the test agent or compound.

In the present invention, the therapeutic effect may be correlated with the expression level of the E2F1 or E2F2 gene. For example, when the test agent or compound reduces the expression level of the E2F1 or E2F2 gene as compared to a level detected in the absence of the test agent or compound, the test agent or compound may identified or selected as the candidate agent or compound having the therapeutic effect. Alternatively, when the test agent or compound does not reduce the expression level of the E2F1 or E2F2 gene as compared to a level detected in the absence of the test agent or compound, the test agent or compound may be identified as the agent or compound having no significant therapeutic effect.

The above steps are same as the method using the promoting activity of E2F1 gene and E2F2 gene expression as a biological activity of JARID1B polypeptide described in “Screening for an agent or compound suppressing the biological activity of JARID1B”. However, in the present method, the expression level of JARID1B gene is focused. When E2F reporter assay is used for detection of the expression level of JARID1B gene, an E2F reporter vector may be introduced into the cell before or after contacting with the test agent or compound.

In the present invention, it is revealed that suppressing the expression of JARID1B, reduces cell growth. Further, it is revealed that the expression of E2F1 gene and E2F2 gene were suppressed by inhibition of JARID1B gene expression. Thus, by screening for candidate compounds that inhibits the expression level of the E2F1 or E2F2 gene, candidate compounds that have the potential to treat or prevent cancers can be identified. Potential of these candidate compounds to treat or prevent cancers may be evaluated by second and/or further screening to identify therapeutic agent or compound for cancers.

Alternatively, the screening method of the present invention may include the following steps:

(a) contacting a test agent or compound with a cell into which a vector, including the transcriptional regulatory region of JARID1B and a reporter gene that is expressed under the control of the transcriptional regulatory region, has been introduced;

(b) measuring the expression or activity of said reporter gene; and

(c) selecting the test compound that reduces the expression or activity of said reporter gene as compared to the expression or activity level in the absence of the test agent or compound as a candidate agent or compound.

According to the present invention, the therapeutic effect of the test agent or compound on inhibiting the cell growth or a candidate agent or compound for treating or preventing JARID1B associating cancer may be evaluated. Therefore, the present invention also provides a method for screening a candidate agent or compound that suppresses the proliferation of cancer cells, and a method for screening a candidate agent or compound for treating or preventing JARID1B associating cancer.

According to another aspect, the present invention provides a method which includes the following steps of:

a) contacting a test agent or compound with a cell into which a vector, composed of the transcriptional regulatory region of the JARID1B gene and a reporter gene that is expressed under the control of the transcriptional regulatory region, has been introduced;

b) detecting the expression level or activity of said reporter gene; and

c) correlating the expression level of b) with the therapeutic effect of the test agent or compound.

In the present invention, the therapeutic effect may be correlated with the expression level or activity of said reporter gene. For example, when the test agent or compound reduces the expression level or activity of said reporter gene as compared to a level detected in the absence of the test agent or compound, the test agent or compound may be identified or selected as the candidate agent or compound having the therapeutic effect. Alternatively, when the test agent or compound does not reduce the expression level or activity of said reporter gene as compared to a level detected in the absence of the test agent or compound, the test agent or compound may be identified as the agent or compound having no significant therapeutic effect.

Suitable reporter genes and host cells are well-known in the art. For example, reporter genes are luciferase, green florescence protein (GFP), Discosoma sp. Red Fluorescent Protein (DsRed), Chrolamphenicol Acetyltransferase (CAT), lacZ and betaglucuronidase (GUS), and host cell is COS7, HEK293, HeLa and so on. The reporter construct required for the screening can be prepared by connecting reporter gene sequence to the transcriptional regulatory region of the JAEID1B gene. The transcriptional regulatory region of JARID1B gene herein is the region from start codon to at least 500 bp upstream, preferably 1000 bp, more preferably 5000 or 10000 bp upstream. A nucleotide segment containing the transcriptional regulatory region can be isolated from a genome library or can be propagated by PCR. The reporter construct required for the screening can be prepared by connecting reporter gene sequence to the transcriptional regulatory region of any one of these genes. Methods for identifying a transcriptional regulatory region, and also assay protocol are well known (Molecular Cloning third edition chapter 17, 2001, Cold Springs Harbor Laboratory Press). For example, the transcriptional regulatory region of JARID1B was reported in Catteau et al. Int J Oncol. 2004, 25: 5-16.

The vector containing the said reporter construct is infected to host cells and the expression or activity of the reporter gene is detected by method well known in the art (e.g., using luminometer, absorption spectrometer, flow cytometer and so on). “reduces the expression or activity” as defined herein are preferably at least 10% reduction of the expression or activity of the reporter gene in comparison with in absence of the compound, more preferably at least 25%, 50% or 75% reduction and most preferably at 95% reduction.

In the present invention, it is revealed that suppressing the expression of JARID1B, reduces cell growth. Thus, by screening for candidate compounds that the expression or activity of said reporter gene, candidate compounds that have the potential to treat or prevent cancers can be identified. Potential of these candidate compounds to treat or prevent cancers may be evaluated by second and/or further screening to identify therapeutic agent or compound for cancers.

Methods for Altering the Expression of E2F1 and E2F2:

According to the present invention, the expression of E2F1 gene and E2F2 gene were suppressed by inhibition of JARID1B gene expression (FIG. 9). It indicates that E2F1 gene and E2F2 gene are candidate downstream genes affected by JARID1B protein.

The E2F transcription factors are downstream effectors of the retinoblastoma (RB) protein pathway and are involved in many aspects of fundamental cell cycle control (Botz J. et al. Mol Cell Biol 1996;16: 3401-9, DeGregori J. et al. Genes Dev 1995;9: 2873-87, Johnson DG et al. Nature 1993;365: 349-52, Muller H. et al. Biochim Biophys Acta 2000;1470: M1-12). Binding sites for E2F factors have been identified in a large number of genes that control cell cycle and DNA synthesis, including cdk2 and 4, cyclin A, D and E, DNA polymerase, ribonucleotide reductase and PCNA (Yamasaki L. Results Probl Cell Differ 1998;22: 199-227). Importantly, mutations in the RB-E2F cascade are found in a wide range of various tumor entities (Dimova D K et al. Oncogene 2005;24: 2810-26, Nevins J R. Hum Mol Genet 2001;10: 699-703).

Whereas most of these alterations affect RB or upstream regulators of the E2F transcriptional factors, there is growing evidence that dysregulation of the E2F family itself is crucially involved in carcinogenesis. Indeed, in ovarian cancer, the proliferation-promoting E2F1 and especially the E2F2 transcription factors were overexpressed, compared with healthy control tissue (Reimer D. et al. Clin Cancer Res 2007;13: 144-51). Additionally, dysregulation of these cell cycle promoting transcriptional factors has been described as a prognostic indicator in various tumors (Ebihara Y. et al. Dis Esophagus 2004;17: 150-4, Foster CS et al. Oncogene 2004;23: 5871-9, Gorgoulis V G et al. J Pathol 2002;198: 142-56, Mega S. et al. Dis Esophagus 2005;18: 109-13, Oeggerli M. et al. Oncogene 2004;23: 5616-23). Therefore, the overexpression of a proliferation-promoting E2F transcription factor could contribute to a significant growth advantage of tumors, contributing to poor survival. In the present invention, the expression levels of both E2F1 and E2F2 gene in bladder tumor tissues are also significantly higher than in nonneoplastic tissues. Dysregulation of E2F/RB pathway can closely links human carcinogenesis in a variety of tissues, and JARID1B may make a contribution to the malignant alterations through deregulating this pathway.

Therefore, the present invention also provides the method for altering the expression level of E2F1 gene and E2F2 gene by regulating JARID1B gene expression or JARID1B protein activity. The present method may contribute to development of new cancer therapy as E2F1 and E2F2 dysregulation is related to carcinogenesis.

In the context of the present invention, the method for altering the expression level of E2F1 gene and E2F2 gene includes the step of altering the expression level of JARID1B gene or the activity of JARID1B protein in a cell. Such step is performed by administering a compound modulating JARID1B gene expression or JARID1B protein activity to a cell. Such compounds can be inhibitors or activators against JARID1B gene expression or JARID1B protein, preferably, inhibitors screened by the aforementioned screening methods. For example, antisense RNAs or double-stranded molecules targeting JARID1B gene, or antibodies against JARID1B protein can be used for the present method. When double-stranded molecules are used for the present method, the double-stranded molecules of the present invention are available. Preferably, such double-stranded molecule comprises the target sequence corresponding to SEQ ID NOs: 21 or 30.

In another embodiment, present invention also provides compositions for altering the expression of E2F1 gene or E2F2 gene, which comprises a compound modulating JARID1B gene expression or JARID1B protein activity. Such compositions comprise at least one compound which inhibits or promote JARID1B gene expression or JARID1B protein activity. Preferably, the composition comprises the double-stranded molecule of the present invention which inhibits JARID1B gene expression or the vector encoding thereof, more preferably, the double stranded-molecule comprising the target sequence corresponding to SEQ ID NOs: 21 or 30.

In the present invention, it is revealed that suppressing the expression of JARID1B, reduces cell growth. Further, it is revealed that the expression of E2F1 gene and E2F2 gene were suppressed by inhibition of JARID1B gene expression. Thus, by screening for candidate compounds that alters the expression level of the E2F1 or E2F2 gene, candidate compounds that have the potential to treat or prevent cancers can be identified. Potential of these candidate compounds to treat or prevent cancers may be evaluated by second and/or further screening to identify therapeutic agent or compound for cancers.

Aspects of the present invention are described in the following examples, which are not intended to limit the scope of the invention described in the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

The invention will be further described in the following examples, which do not limit the scope of the invention described in the claims.

### EXAMPLES

The invention will be further described in the following examples, which do not limit the scope of the invention described in the claims.

### Example 1

General Methods

Tissue Samples and RNA Preparation.

123 surgical specimens of primary urothelial carcinoma were collected, either at cystectomy or transurethral resection of bladder tumor (TUR-Bt), and snap frozen in liquid nitrogen. 23 specimens of normal bladder urothelial tissue were collected from areas of macroscopically normal bladder urothelium in patients with no evidence of malignancy. Use of tissues for this study was approved by Cambridge shire Local Research Ethics Committee. A total of thirty 30-micrometer sections were homogenized for RNA extraction and two 7- micrometer ‘sandwich’ sections adjacent to the tissue used for RNA extraction were sectioned, stained and assessed for cellularity and tumor grade by an independent consultant urohistopathologist. Additionally, the sections were graded according to the degree of inflammatory cell infiltration (low, moderate and significant). Samples showing significant inflammatory cell infiltration were excluded (Wallard M J et al. British journal of cancer 2006;94: 569-77). Total RNA was extracted using TRI Reagent™ (Sigma, Dorset, UK), following the manufacturers protocol. RNeasy Minikits™ (QIAGEN, Crawley, UK), including a DNase step, was used to optimize RNA purity. Agilent 2100™ total RNA bioanalysis was performed. 1 microliter of resuspended RNA from each sample was applied to an RNA 6000 Nano Lab Chip™ and processed according to the manufacturer's instructions. All chips and reagents were sourced from Agilent Technologies™ (West Lothian, UK).

Reverse Transcription.

Total RNA concentrations were determined using the NanoDrop™ ND 1000 spectrophotometer (Nyxor Biotech, Paris, France). 1 microgram of total RNA was reverse transcribed with 2 microgram random hexamers (Amersham) and Superscript III reverse transcriptase (Invitrogen, Paisley, UK) in 20 microliter reactions according to the manufacturer's instructions. cDNA was then diluted 1:100 with PCR grade water and stored at −20 degrees C.

Laser Capture Microdissection.

Tissue for laser capture microdissection was collected prospectively following the procedure outlined above. Five sequential sections of 7-micrometer thickness were cut from each tissue and stained using Histogene™ staining solution (Arcturus, Calif., USA) following the manufacturer's protocol. Slides were then immediately transferred for microdissection using a PixCell II laser capture microscope™ (Arcturus, Calif., USA). This technique employs a low-power infrared laser to melt a thermoplastic film over the cells of interest, to which the cells become attached.

Approximately 10000 cells were microdissected from both stromal and epithelial/tumor compartments in each tissue. RNA was extracted using an RNeasy Micro Kit (QIAGEN, Crawley, UK). Areas of cancer or stroma containing significant inflammatory areas of tumor or stroma containing significant inflammatory cell infiltration were avoided to prevent contamination.

Total RNA was reverse transcribed and qRT-PCR performed as shown above. Given the low yield of RNA from such small samples, NanoDrop™ quantification was not performed, but correction for the endogenous 18S CT value was used as an accurate measure of the amount of intact starting RNA. Transcript analysis was performed for JARID1B gene.

To validate the accuracy of microdissection, qRT-PCT using primers and probes for Vimentin and Uroplakin were performed according to the manufacturer's instructions (Assays on demand, Applied Biosystems, Warrington, UK). Vimentin is primarily expressed in mesenchymal tissue, and was used as a stromal marker. Uroplakin is a marker of urothelial differentiation and is preserved in up to 90% of epithelially-derived tumors (Olsburgh J. et al. The Journal of pathology 2003;199: 41-9).

Cell Culture.

All cell lines were grown in monolayers in appropriate media: Eagle's minimal essential medium (EMEM) for 253J, 253J-BV, HT1197, HT1376, J82, SCaBER, UMUC3 bladder cancer cells and SBC5 small cell lung cancer cells; RPMI1640 medium for 5637 bladder cancer cells and A549, H2170 and LC319 non-small cell lung cancer cells; Dulbecco's modified Eagle's medium (DMEM) for EJ28 bladder cancer cells and RERF-LC-AI non-small cell lung cancer cells; McCoy's 5A medium for RT4 and T24 bladder cancer cells; Leibovitz's L-15 for SW780 cells supplemented with 10% fetal bovine serum and 1% antibiotic/antimycotic solution (Sigma). All cells were maintained at 37 degrees C. in humid air with 5% CO2, (253J, 253J-BV, HT1197, HT1376, J82, SCaBER, UMUC3, SBC5, 5637, A549 H2170, LC319, EJ28, RERF-LC-AI, RT4 and T24) or without CO2 (SW780). Cells were transfected with FuGENE6 (ROCHE, Basel, Switzerland) according to manufacturers' protocols.

Expression Profiling in Cancer Using cDNA Microarrays.

The present inventors had established a genome-wide cDNA microarray with 36,864 cDNAs or ESRs selected from the UniGene database of the National Center for Biotechnology Information (NCBI). This microarray system was constructed essentially as described previously (Kikuchi T. et al. Oncogene 2003;22: 2192-205, Kitahara O. et al. Cancer Res 2001;61: 3544-9, Nakamura T. et al. Oncogene 2004;23: 2385-400). Briefly, the cDNAs were amplified by RT-PCR using poly (A)⁺ RNAs isolated from various human organs as templates; the lengths of the amplicons ranged from 200 to 1,100 bp, without any repetitive or poly (A) sequences. Many types of tumors and corresponding non-neoplastic tissues were prepared in 8-micrometer sections, as described previously (Kitahara O. et al. Cancer Res 2001;61: 3544-9). A total of 30,000-40,000 cancer or noncancerous cells were collected selectively using the EZ cut system (SL Microtest GmbH, Germany) according to the manufacturer's protocol. Extraction of total RNA, T7-based amplification, and labeling of probes were performed as described previously (Kitahara O. et al. Cancer Res 2001;61: 3544-9). A measure of 2.5-microgram aliquots of twice-amplified RNA (aRNA) from each cancerous and noncancerous tissue were then labeled, respectively, with Cy3-dCTP or Cy5-dCTP.

Quantitative Real-Time PCR.

As described above, 123 bladder cancer tissues and normal 23 bladder tissues were prepared in Cambridge Addenbrooke's Hospital. For quantitative RT-PCR reactions, specific primers for all human GAPDH (housekeeping gene), SDH (housekeeping gene), JARID1B, E2F1 and E2F2 were designed as follows:

(SEQ ID NOs: 3)
5′ GCAAATTCCATGGCACCGTC 3′
for GAPDH-foward;
(SEQ ID NOs: 4)
5′ TCGCCCCACTTGATTTTGG 3′
for GAPDH-reverse;
(SEQ ID NOs: 5)
5′ TGGGAACAAGAGGGCATCTG 3′
for SDH-foward;
(SEQ ID NOs: 6)
5′ CCACCACTGCATCAAATTCATG3′
for SDH-reverse;
(SEQ ID NOs: 7)
5′ ATTGCCTCAAAGGAATTTGGCAGTG3′
for JARID1B-forward-1;
(SEQ ID NOs: 8)
5′ CATCACTGGCATGTTGTTCAAATTC 3′
for JARID1B-reverse-1;
(SEQ ID NOs: 9)
5′ TGTCACAGTGGAATATGGAGCTGAC 3′
for JARID1B-foward-2;
and
(SEQ ID NOs: 10)
5′ GCCACTATCAAGATACTCCTCTTCC 3′
for JARID1B-reverse-2;
(SEQ ID NOs: 11)
5′ GCTGGACCACCTGATGAATATC 3′
for E2F1-foward;
(SEQ ID NOs: 12)
5′ TCTGCAATGCTACGAAGGTCCTG 3′
for E2F1-reverse;
(SEQ ID NOs: 13)
5′ TGGCAACTTTAAGGAGCAGACAG 3′
for E2F2-forward;
(SEQ ID NOs: 14)
5′ GGGCACAGGTAGACTTCGATGG 3′
for E2F2-reverse.

PCR reactions were performed using the ABI prism 7700 Sequence Detection System (Applied Biosystems, Warrington, UK) following the manufactures' protocol. 50% SYBR GREEN universal PCR Master Mix without UNG (Applied Biosystems, Warrington, UK), 50 nM each of the forward and reverse primers and 2 microliter of reversely-transcribed cDNA were applied. Amplification conditions were firstly 5 min at 95 degrees C. and then 45 cycles each consisting of 10 sec at 95 degrees C., 1 min at 55 degrees C. and 10 sec at 72 degrees C. Then, reactions were heated for 15 sec at 95 degrees C., 1 min at 65 degrees C. to draw the melting curve, and cooled to 50 degrees C. for 10 sec. Reaction conditions for target gene amplification were as described above and the equivalent of 5 ng of reverse transcribed RNA was used in each reaction. mRNA levels were normalized to GAPDH and SDH expressions.

Immunocytochemistry.

A549 (non-small cell lung cancer) or SBC5 (small cell lung cancer) cells were fixed with PBS(−) containing 4% paraformaldehyde for 20 min, and rendered permeable with PBS(−) containing 0.1% Triton X-100 at room temperature for 2 min. Subsequently, the cells were covered with 3% bovine serum albumin in PBS(−) for 1 h at room temperature to block nonspecific hybridization, and then were incubated mouse anti-JARID1B antibody (1G10, Abnova) at 1:100 ratio dilution. After washing with PBS(−), cells were stained by an Alexa594-conjugated anti-mouse secondary antibody (Molecular Probe) at 1:1000 dilutions. Nuclei were counter-stained with 4′,6′-diamidine-2′-phenylindole dihydrochloride (DAPI). Fluorescent images were obtained under a TCS SP2 AOBS microscope (Leica).

Immunohistochemical Staining

Sections of human bladder and lung cancer were stained by VECTASTAIN™ ABC KIT (VECTOR LABORATORIES, CA, USA). Briefly, endogenous peroxidase activity of xylene-deparaffinized and dehydrated sections was inhibited by treatment with 0.3% H₂O₂/methanol. Non-specific binding was blocked by incubating sections with 3% BSA in a humidified chamber for 30 min at ambient temperature followed by overnight incubation at 4 degrees C. with a 1:100 dilution of mouse monoclonal anti-JARID1B (clone 1G10, Abnova) antibody. The sections were washed twice with PBS (−), incubated with 5 microgram/microlitter goat anti-mouse biotinylated IgG in PBS (−) containing 1% BSA for 30 min at ambient temperature, and then incubated with ABC reagent for 30 min. Specific immunostaining was visualized by 3,3′-diaminobenzidine. Slides were dehydrated through graded alcohol to xylene washing and mounted on cover slips. Hematoxylin was used for nuclear counter-staining.

Western Blotting

Total protein extracts were prepared from the cells in RIPA-like buffer. Total protein (50 microgram) was transferred to nitrocellulose membrane. The membrane was proved with anti-JARID1B (clone 1G10, Abnova or HPA027179, Atlas Antibodies AB), anti-E2F1 antibody (KH-95, Santa Cruz Biotechnology) and anti-E2F2 antibody (L-20, Santa Cruz Biotechnology). Anti-Actin (1-19, Santa Cruz Biotechnology) was used as loading control.

Transfection with siRNAs.

siRNA oligonucleotide duplexes were purchased from SIGMA Genosys for targeting the human JARID1B transcript. siEGFP, siFFLuc and siNegative control (siNC), which is a mixture of three different oligonucleotide duplexes, were used as control siRNAs. The siRNA sequences are shown as follows.

(SEQ ID NOs: 15)
5′ GCAGCACGACUUCUUCAAGTT 3′
for siEGFP sense;
(SEQ ID NOs: 16)
5′ CUUGAAGAAGUCGUGCUGCTT 3′
for siEGFP antisense;
(SEQ ID NOs: 17)
5′ GUGCGCUGCUGGUGCCAACTT 3′
for siFFLuc sense;
(SEQ ID NOs: 18)
5′ GUUGGCACCAGCAGCGCACTT 3′
for siFFLuc antisense;
(SEQ ID NOs: 22)
5′ AUCCGCGCGAUAGUACGUA 3′
for siNegative control Target#1 sense;
(SEQ ID NOs: 23)
5′ UACGUACUAUCGCGCGGAU 3′
for siNegative control Target#1 antisense;
(SEQ ID NOs: 24)
5′ UUACGCGUAGCGUAAUACG 3′
for siNegative control Target#2 sense;
(SEQ ID NOs: 25)
5′ CGUAUUACGCUACGCGUAA 3′
for siNegative control Target#2 antisense;
(SEQ ID NOs: 26)
5′ UAUUCGCGCGUAUAGCGGU 3′
for siNegative control Target#3 sense;
(SEQ ID NOs: 27)
5′ ACCGCUAUACGCGCGAAUA 3′
for siNegative control Target#3 antisense;
(SEQ ID NOs: 19)
5′ CAGUGAAUGAGCUCCGGCATT 3′
for JARID1B#1 sense;
(SEQ ID NOs: 20)
5′ UGCCGGAGCUCAUUCACUGTT 3′
for JARID1B#1 antisense;
(SEQ ID NOs: 28)
5′ GGAAUAUGGAGCUGACAUUTT 3′
for JARID1B#2 sense;
and
(SEQ ID NOs: 29)
5′ AAUGUCAGCUCCAUAUUCCTT 3′
for JARID1B#2 antisense.

siRNA duplexes (100 nM final concentration) were transfected in bladder and lung cancer cell lines with lipofectamine2000 (Invitrogen) for 72 hours, and cell viability was examined using Cell Counting kit 8 (DOJINDO Laboratories).

Flow Cytometry Assays (FACS).

To examine the effect of JARID1B expression on the cell cycle progression, SBC5 cells were treated with siJARID1Bs or control siRNAs (siEGFP and siFFLuc), and cultured in a CO₂ incubator at 37 degrees C. for 72 hours. 1×10⁵ cells were collected by trypsinization, and stained with propidium iodide following the manufacturer's instructions (Cayman Chemical). Cells were analyzed by FACScan (BECKMAN COULTER) with MultiCycle for Windows software (BECKMAN COULTER) for detailed cell cycle status. The percentages of cells in G0/G1, S and G2/M phases of the cell cycle, and any sub-G1 population, were determined from at least 20,000 ungated cells.

Microarray Hybridization and Statistical Analysis for the Clarification of Down-Stream Genes.

Purified total RNA was labeled and hybridized onto Affymetrix GeneChip U133 Plus 2.0 oligonucleotide arrays (Affymetrix, Santa Clara, Calif.) according to the manufacturer's instructions.

Hybridization signals were scaled in the Affymetrix GCOS software (version 1.1.1) using a scaling factor determined by adjusting the global trimmed mean signal intensity value to 500 for each array and imported into GeneSpring version 6.2 (Silicon Genetics). Signal intensities were then centered to the 50th percentile of each chip and, for each individual gene, to the median intensity of each specific subset first to minimize the possible technical bias and then to the whole sample set. The intensity of any replicate hybridizations were averaged subsequent to further analysis. Only genes labeled by the GCOS software as “present” or “marginal” in all samples were used for further analysis. Differentially expressed genes were identified using the Wilcoxon-Mann-Whitney nonparametric test (P<0.05). The Benjamini-Hochberg false discovery rate multiple test correction was applied whenever applicable. Hierarchical cluster analysis was done on each comparison to assess correlations among samples for each identified gene set.

Alternatively, probe signal intensities were normalized by RMA and Quantile normalization methods (using R and Bioconductor). Next, signal intensity fluctuation due to inter-experimental variation was estimated. Each experiment was replicated (1 and 2), and the standard deviation (stdev) of log 2(intensity2/intensity 1) was calculated for each of a set of intensity ranges with the midpoints being at log 2((intensity 1+intensity2)/2)=5, 7, 9, 11, 13, and 15. Intensity variation was modeled using the formula stdev(log 2(intensity2/intensity1))=a*(log 2((intensity 1+intensity2)/2))+b and parameters a and b were estimated using the method of least squares. Using these values, the standard deviation of intensity fluctuation was calculated. The signal intensities of each probe were then compared between siJARID1B (EXP) and controls (EGFP/FFLuc) (CONT) and tested for up/down-regulation by calculating the z-score: log2(intensityEXP/intensityCONT)/(a*(log 2((intensityEXP+intensityCONT)/2))+b). Resultant P-values for the replication sets were multiplied to calculate the final P-value of each probe. These procedures were applied to each comparison: EGFP vs. siJARID1B, FFLuc vs. siJARID1B, and EGFP vs. FFLuc, respectively. Up/down-regulated gene sets were determined as those that simultaneously satisfied the following criteria: (1) The Benjamin-Hochberg false discovery rate (FDR)<=0.05 for EGFP vs. siJARID1B, (2) FDR<=0.05 for FFLuc vs. siJARID1B and the regulation direction is the same as (1), and (3) EGFP vs. FFLuc has the direction opposite to (1) and (2) or P>0.05 for EGFP vs. FFLuc. Finally, a pathway analysis was performed using the hyper-geometric distribution test, which calculates the probability of overlap between the up/down-regulated gene set and each GO category compared against another gene list that is randomly sampled. The test was applied to the identified up/down-regulated genes to test whether or not they are significantly enriched (FDR<=0.05) in each category of “Biological processes” (857 categories) as defined by the Gene Ontology database.

E2F Reporter Assay.

The transcriptional activity of E2F was analyzed by the Cignal™ E2F Reporter Assay Kit (SuperArray Bioscience Corporation). A549 and SBC5 cells were treated with siRNAs (siJARID1Bs, siEGFP and siFFLuc) and cultured for 24 hours. siRNA-treated cells were transfected with E2F-responsive luciferase construct, which encodes the firefly luciferase reporter gene under the control of a minical (m) CMV promoter and tandem repeats of the E2F transcriptional response element (TRE), negative control or positive control. After 24 hours of transfection, dual luciferase assay was performed using Dual-Luciferase Reporter Assay System (Promega), and promoter activity values are expressed as arbitrary units using a Renilla reporter for internal normalization. Experiments were done in triplicate, and Student's t-test was used as statistical analysis.

### Example 2

JARID1B Expression is Up-Regulated in Clinical Cancer Tissues.

First, expression levels of five histone demethylase genes, JARID1A, JARID1B, JARID1C, JARID1D and JARID2, were examined in a small subset of clinical bladder cancer samples and were found significant difference in expression levels between normal and cancer cells only for the JARID1B gene (data not shown). In order to verify how the expression levels of JmjC histone demethylases change in malignant tumors, expression levels of five histone demethylase genes, JARID1A, JARID1B, JARID1C, JARID1D and JARID2, were examined using quantitative real-time PCR in a small subset of clinical bladder cancer samples and were found significant difference in expression levels between normal and cancer cells only for the JARID1B gene (data not shown). Therefore, 123 bladder cancer samples and 23 normal control samples (British) were analyzed (FIGS. 1A and B), and significant overexpression of JARID1B in tumor cells as compared with in normal tissues was observed (P<0.0001, Mann-Whitney U-test, FIG. 1B, Table 1).

TABLE 1
Clinicopathologic characteristics and JARID1B expression

| Tissue | Sample name | JARID1B expression | pT | Grade |
|---|---|---|---|---|
| Tumor | BT2 | 3.723757677 | T4 | 3 |
| | BT5 | 5.133204008 | Ta | uk |
| | BT6 | 4.188371931 | Ta | 2 |
| | BT8 | 2.392817477 | Ta | 2 |
| | BT9 | 4.383047299 | Ta | 2 |
| | BT10 | 5.688445406 | T2 | 3 |
| | BT11 | 3.631539584 | T2 | 3 |
| | BT12 | 3.378164362 | T1 | 2 |
| | BT15 | 4.513809549 | T2 | 3 |
| | BT16 | 3.307031343 | Ta | 2 |
| | BT18 | 3.225786495 | Ta | 3 |
| | BT20 | 7.264818254 | T1 | 2 |
| | BT21 | 3.73186917 | Ta | 3 |
| | BT22 | 5.96353482 | T2 | 2 |
| | BT23 | 4.124373502 | T1 | 2 |
| | BT28 | 3.171562984 | Ta | 1 |
| | BT31 | 8.472334982 | Ta | 2 |
| | BT32 | 1.178595838 | T2 | 3 |
| | BT33 | 2.01221958 | T1 | 2 |
| | BT34 | 5.172175761 | Ta | 2 |
| | BT35 | 3.321182379 | T3a | 3 |
| | BT36 | 2.481706615 | T2 | 3 |
| | BT38 | 4.937286373 | Ta | 2 |
| | BT39 | 5.87070444 | T1 | 3 |
| | BT40 | 1.368281641 | T2 | 3 |
| | BT41 | 1.14384635 | T1 | 2 |
| | BT42 | 2.254269456 | T2 | 3 |
| | BT43 | 5.80078388 | Ta | 1 |
| | BT44 | 8.624782522 | T1 | 2 |
| | BT46 | 3.684335203 | Ta | 2 |
| | BT48 | 7.861600554 | T2 | 3 |
| | BT49 | 4.282269434 | Ta | 1 |
| | BT50 | 8.482319486 | T1 | 3 |
| | BT51 | 4.541882865 | Ta | 2 |
| | BT52 | 6.174337486 | T3 | 3 |
| | BT53 | 7.942166599 | Ta | 2 |
| | BT54 | 1.688164538 | T1 | 3 |
| | BT56 | 3.464832277 | T2 | 3 |
| | BT57 | 4.616693966 | T1 | 2 |
| | BT58 | 2.674320995 | Ta | 2 |
| | BT59 | 3.932299671 | T2 | 3 |
| | BT60 | 8.114636683 | Mets | 3 |
| | BT64 | 8.550992233 | Ta | 2 |
| | BT66 | 4.093128255 | Ta | 1 |
| | BT67 | 5.058858835 | T1 | 2 |
| | BT68 | 3.670499567 | Ta | 2 |
| | BT69 | 24.88108906 | Ta | 2 |
| | BT70 | 6.05734334 | T1 | 2 |
| | BT71 | 4.133922672 | T1 | 3 |
| | BT72 | 8.458194325 | Ta | 1 |
| | BT74 | 5.777832954 | Ta | 1 |
| | BT76 | 3.293270361 | T1 | 1 |
| | BT77 | 4.147895826 | Ta | 2 |
| | BT78 | 2.834558288 | T1 | 3 |
| | BT79 | 2.537326226 | Ta | 2 |
| | BT80 | 6.609713052 | Ta | 2 |
| | BT81 | 3.010341953 | Ta | 2 |
| | BT82 | 3.730894533 | T1 | 3 |
| | BT83 | 8.337717858 | Ta | 2 |
| | BT84 | 10.6155221 | Ta | 2 |
| | BT85 | 3.972433124 | T1 | 2 |
| | BT87 | 0.525080141 | T2 | 2 |
| | BT88 | 10.60653495 | T1 | 3 |
| | BT90 | 5.899961936 | Ta | 2 |
| | BT92 | 3.434195352 | T1 | 2 |
| | BT93 | 2.468130141 | T2 | 3 |
| | BT94 | 10.17964275 | Ta | 1 |
| | BT95 | 2.382499274 | T3a | 3 |
| | BT96 | 3.358390417 | Ta | 1 |
| | BT97 | 7.767841254 | Ta | 2 |
| | BT98 | 15.44824773 | Ta | 2 |
| | BT99 | 9.024065187 | T1 | 2 |
| | BT100 | 7.666354252 | T1 | 3 |
| | BT101 | 6.639631354 | T2 | 3 |
| | BT103 | 8.359395135 | T1 | 2 |
| | BT104 | 4.970020969 | T4 | 2 |
| | BT105 | 4.013647709 | T2 | 2 |
| | BT106 | 4.197667938 | Ta | 3 |
| | BT107 | 2.985594674 | Mets | 3 |
| | BT108 | 17.79609597 | T1 | 2 |
| | BT109 | 4.178500086 | Ta | 2 |
| | BT110 | 3.897383041 | T1 | 2 |
| | BT112 | 6.820473729 | Ta | 3 |
| | BT113 | 3.885112471 | T1 | 3 |
| | BT114 | 2.031697472 | T2 | 3 |
| | BT115 | 2.196992553 | T1 | 3 |
| | BT116 | 5.575781632 | T2a | 3 |
| | BT119 | 6.928136024 | Ta | 2 |
| | BT120 | 10.88107536 | Ta | 2 |
| | BT122 | 4.566211985 | T1 | 3 |
| | BT125 | 7.654271815 | T1 | 2 |
| | BT127 | 7.136781851 | T1 | 2 |
| | BT128 | 7.894791474 | Ta | 1 |
| | BT129 | 3.257744612 | Ta | 2 |
| | BT130 | 5.014176867 | Ta | 2 |
| | BT131 | 4.813191905 | T2 | 3 |
| | BT132 | 3.100918885 | T2 | 3 |
| | BT133 | 3.500649848 | T1 | 2 |
| | BT135 | 3.160757145 | T2 | 3 |
| | BT137 | 2.894619193 | Ta | 2 |
| | BT138 | 1.21990425 | Ta | 1 |
| | BT139 | 2.177775824 | T2 | 3 |
| | BT140 | 5.729698929 | Ta | 2 |
| | BT141 | 2.063801268 | Mets | 3 |
| | BT143 | 2.82525806 | T1 | 3 |
| | BT145 | 3.626263347 | T2 | 2 |
| | BT150 | 0.96273313 | Ta | 2 |
| | BT151 | 3.69777386 | Ta | 3 |
| | BT152 | 2.168371128 | Ta | 2 |
| | BT154 | 7.815917508 | T1 | 3 |
| | BT158 | 4.685174508 | Ta | 2 |
| | BT160 | 3.651623011 | T1 | 2 |
| | BT161 | 7.75780893 | Ta | 2 |
| | BT162 | 1.139458522 | T3 | 3 |
| | BT164 | 2.884535653 | Ta | 1 |
| | BT165 | 2.164550933 | T2 | 3 |
| | BT169 | 0.965883667 | T2 | 3 |
| | BT178 | 2.990188871 | Ta | 2 |
| | BT180 | 2.962694209 | T1 | 2 |
| | BT181 | 3.508136742 | T2 | 3 |
| | BT187 | 8.076148665 | Ta | 2 |
| | BT188 | 4.920557483 | T2 | 3 |
| | BT189 | 4.396314262 | T1 | 2 |
| Normal | BN11A | 2.041077373 | Normal | Normal |
| | BN12A | 1.37836977 | Normal | Normal |
| | BN13A | 1.309847551 | Normal | Normal |
| | BN14A | 1.503884371 | Normal | Normal |
| | BN14B | 1.556433786 | Normal | Normal |
| | BN15A | 2.120084104 | Normal | Normal |
| | BN17B | 1.815168518 | Normal | Normal |
| | BN19A | 1.774206371 | Normal | Normal |
| | BN1A | 2.793568931 | Normal | Normal |
| | BN20B | 1.875164786 | Normal | Normal |
| | BN21A | 3.430175314 | Normal | Normal |
| | BN22A | 1.133256753 | Normal | Normal |
| | BN22B | 1.759057821 | Normal | Normal |
| | BN25A | 2.119878568 | Normal | Normal |
| | BN26A | 0.997744917 | Normal | Normal |
| | BN2A | 2.367327592 | Normal | Normal |
| | BN2B | 2.440497064 | Normal | Normal |
| | BN4A | 2.441758365 | Normal | Normal |
| | BN4B | 2.064467556 | Normal | Normal |
| | BN5B | 1.694005395 | Normal | Normal |
| | BN6A | 2.011887643 | Normal | Normal |
| | BN8A | 1.644228733 | Normal | Normal |
| | BN9A | 2.237320664 | Normal | Normal |

No significant difference was observed in expression levels among different stages and grades (FIG. 1A and Table 1, 2). This suggests that JARID1B expression is significantly up-regulated in an early stages of bladder carcinogenesis, and remains high in the advanced stages of the disease. Subclassification of tumors according to metastasis status, gender, smoking history and recurrence status identified no significant difference in the expression levels of JARID1B (Table 2).

TABLE 2
Statistical analysis of JARID1B expression levels in
clinical bladder tissues.

JARID1B

| Characteristic | n | Mean | SD | 95% CI |
|---|---|---|---|---|
| Normal (Control) | 23 | 1.935 | 0.549 | 1.698-2.173 |
| Tumor (Total) | 123 | 5.018 | 3.322 | 4.425-13.025 |
| Tumor grade | | | | |
| G1 | 12 | 5.035 | 2.654 | 3.349-6.720 |
| G2 | 62 | 5.740 | 3.982 | 4.728-6.751 |
| G3 | 48 | 4.079 | 2.196 | 3.441-4.717 |
| Metastasis | | | | |
| Negative | 96 | 4.988 | 3.547 | 4.269-5.706 |
| Positive | 27 | 5.125 | 2.408 | 4.173-6.078 |
| Gender | | | | |
| Male | 90 | 5.032 | 3.378 | 4.324-5.739 |
| Female | 31 | 4.477 | 2.243 | 3.654-5.299 |
| Smoke | | | | |
| No | 27 | 4.702 | 2.246 | 3.813-5.590 |
| Yes | 49 | 5.549 | 4.050 | 4.386-6.712 |
| Recurrence | | | | |
| No | 27 | 5.606 | 4.735 | 3.733-7.480 |
| Yes | 50 | 4.988 | 2.540 | 4.266-5.710 |
| Died | 8 | 5.948 | 3.280 | 3.205-8.690 |

Then, the expression patterns of JARID1B in a number of clinical samples derived from Japanese bladder cancer subjects was analyzed by cDNA microarray (FIGS. 1C, 1D, Table 3), and confirmed its significant over-expression (P<0.0001, Mann-Whitney U-test).

TABLE 3
The gene expression profile of JARID1B in cancer tissues.
Expression levels of these genes were analyzed by
cDNA microarray*.

| JARID1B | Ratio (Tumor/Normal) | | | | |
|---|---|---|---|---|---|
| Tissue type | Case (n) | Count > 2 | Count > 3 | Count > 5 | Count > 10 |
| Acute | 56 | 43 | 39 | 22 | 0 |
| myelogenous | | (76.8%) | (69.6%) | (39.3%) | (0%) |
| leukemia | | | | | |
| Bladder | 34 | 21 | 6 | 3 | 0 |
| cancer | | (61.8%) | (17.6%) | (8.8%) | (0%) |
| Breast | 81 | 33 | 11 | 3 | 0 |
| cancer | | (40.7%) | (13.6%) | (3.7%) | (0%) |
| Chronic | 74 | 45 | 24 | 6 | 2 |
| myelogenous | | (60.8%) | (32.4%) | (8.1%) | (2.7%) |
| leukemia | | | | | |
| Cervical | 19 | 16 | 13 | 8 | 1 |
| cancer | | (84.2%) | (68.4%) | (42.1%) | (5.3%) |
| Non-small | 37 | 28 | 18 | 7 | 0 |
| cell lung | | (75.7%) | (48.6%) | (18.9%) | (0%) |
| cancer | | | | | |
| Prostate | 58 | 7 | 1 | 0 | 0 |
| cancer | | (12.1%) | (1.7%) | (0%) | (0%) |
| Renal cell | 24 | 10 | 1 | 0 | 0 |
| carcinoma | | (41.7%) | (4.2%) | (0%) | (0%) |
| Small cell | 15 | 11 | 7 | 6 | 0 |
| lung cancer | | (73.3%) | (46.7%) | (40%) | (0%) |

*We compared the signal intensity of JARID1B between tumor tissues and corresponding non-neoplastic tissues derived from the same patient.

To evaluate protein expression levels of JARID1B in bladder tissues, immunohisto-chemical analysis was performed using anti-JARID1B specific antibody (FIG. 1E). Strong JARID1B staining was observed mainly in the nucleus of malignant cells, but no significant staining was observed in non-neoplastic tissues. To further validate this result, tissue microarray experiments were conducted using 28 bladder tissue sections (FIG. 2 and Table 4), and strong staining was observed in 6 cases, and modest or weak staining was observed in 13 cases. Moreover, no significant relationship between JARID1B protein expression levels and clinicopathologic characteristics was detected, consistent with the real-time PCR results.

TABLE 4
Clinicopathologic charcteristics of bladder tissues on
the tissue microarray

| Case | | | | | |
|---|---|---|---|---|---|
| No. | Gender | Age | Histology | Grade | TNM |
| 1 | Male | 71 | Squamous cell carcinoma | 1 | T1N0M0 |
| 2 | Male | 60 | Squamous cell carcinoma | 1 | T2N0M0 |
| 3 | Male | 76 | Adenocarcinoma | 2 | T2N0M0 |
| 4 | Male | 50 | Adenocarcinoma | 2 | T2N0M0 |
| 5 | Male | 68 | Adenocarcinoma | 3 | T2N0M0 |
| 6 | Female | 74 | Adenocarcinoma | 3 | T2N0M0 |
| 7 | Male | 27 | Transitional cell carcinoma | 1 | TisN0M0 |
| 8 | Male | 50 | Transitional cell carcinoma | 1 | T1N0M0 |
| 9 | Female | 49 | Transitional cell carcinoma | 1 | T1N0M0 |
| 10 | Male | 67 | Transitional cell carcinoma | 1 | T1N0M0 |
| 11 | Female | 51 | Transitional cell carcinoma | 1 | T1N0M0 |
| 12 | Male | 57 | Transitional cell carcinoma | 1 | T1N0M0 |
| 13 | Male | 47 | Transitional cell carcinoma | 2 | T2N0M0 |
| 14 | Male | 54 | Transitional cell carcinoma | 2 | T2N0M0 |
| 15 | Male | 45 | Transitional cell carcinoma | 2 | T1N0M0 |
| 16 | Male | 74 | Transitional cell carcinoma | 2 | T1N0M0 |
| 17 | Male | 51 | Transitional cell carcinoma | 2 | T1N0M0 |
| 18 | Male | 80 | Transitional cell carcinoma | 2 | T2N0M0 |
| 19 | Female | 53 | Transitional cell carcinoma | 2 | T1N0M0 |
| 20 | Male | 37 | Transitional cell carcinoma | 2 | T2N0M0 |
| 21 | Male | 55 | Transitional cell carcinoma | 2 | T2N2MX |
| 22 | Male | 52 | Transitional cell carcinoma | 2 | T1N0M0 |
| 23 | Male | 78 | Transitional cell carcinoma | 3 | T1N0M0 |
| 24 | Male | 64 | Transitional cell carcinoma | 3 | T3N2M1 |
| 25 | Male | 70 | Transitional cell carcinoma | 3 | T2N0M0 |
| 26 | Male | 61 | Transitional cell carcinoma | 3 | T2N0M0 |
| 27 | Male | 61 | Transitional cell carcinoma | 3 | T2N0M0 |
| 28 | Male | 30 | Sarcoma | — | T2N0M0 |

In addition to bladder tissues, expression levels of JARID1B were examined in lung tissues (FIGS. 3, 4). cDNA microarray experiments showed that JARID1B expression was also highly elevated in lung tumor tissues compared with corresponding non-neoplastic tissues (FIGS. 3 A-C). Importantly, elevated JARID1B expression was observed in both non-small cell lung cancers and small cell lung cancers, indicating that JARID1B over-expression is involved widely in lung carcinogenesis. Then, JARID1B protein expression levels were examined in lung tissue by immunohistochemistry (FIG. 3D). Strong JARID1B staining was observed in cancer tissues and no significant staining was observed in non-neoplastic tissues. To evaluate protein expression levels of JARID1B in various types of lung tumor tissues, tissue microarray experiments were conducted (FIG. 4 and Table 5). Among 62 tumor tissue sections examined, strong staining was observed in 54 cases, and modest or weak staining was observed in 9 cases.

TABLE 5
Clinicopathologic charcteristics of lung tissues on the tissue microarray

| Case | | | | Pathological | Tumor | Tumor Size | | |
|---|---|---|---|---|---|---|---|---|
| No. | Gender | Age | Organ | Diagnosis | History | (cm) | Differentiation | TNM |
| 1 | Male | 60 | Lung | Lung metastasis | 3 M | 3 × 3 × 2.5 | Moderately | T2NxM1 |
| | | | | (renal cell | | | | |
| | | | | carcinoma) | | | | |
| 2 | N/A | N/A | Lung | Adenocarcinoma | 1 M | — | — | T0NxMx |
| 3 | N/A | N/A | Lung | Squamous cell | 1 M | — | — | T0NxMx |
| | | | | carcinoma | | | | |
| 4 | Male | 60 | Lung | Squamous cell | 1 M | 5 × 4.5 × 4 | Poorly | T2N0M0 |
| | | | | carcinoma | | | | |
| 5 | Female | 47 | Lung | Adenocarcinoma | 0.5 M | 5 × 4 × 3.5 | Poorly | T2N0M0 |
| 6 | Female | 53 | Lung | Squamous cell | 0.5 M | N/A | Moderately | T0N0M0 |
| | | | | carcinoma | | | | |
| 7 | Male | 40 | Lung | Squamous cell | 5 M | 3.9 × 3.5 × 2.5 | Moderately | T2N0M0 |
| | | | | carcinoma | | | | |
| 8 | Female | 56 | Lung | Adenocarcinoma | 3 M | 3.5 × 3 × 3 | Poorly | T2N0M0 |
| 9 | Male | 49 | Lung | Squamous cell | 12 M | 3.4 × 2.9 × 2.5 | Moderately | T2N0M0 |
| | | | | carcinoma | | | | |
| 10 | Female | 45 | Lung | Bronchio alveolar | 1 Y+ | 4 × 3 × 2 | N/A | T1N0M0 |
| | | | | carcinoma | | | | |
| 11 | Female | 34 | Lung | Fibrosarcoma | 1 M | N/A | Moderately | T0N0M0 |
| 12 | Male | 50 | Lung | Bronchio alveolar | 2 M | 9 × 6.5 × 5 | N/A | T3N0M0 |
| | | | | carcinoma | | | | |
| 13 | Male | 57 | Lung | Squamous cell | 1 M | 5 × 4 × 2.5 | Poorly | T2N0M0 |
| | | | | carcinoma | | | | |
| 14 | Male | 65 | Lung | Atypical Carcinoma | 2 M | 8 × 7 × 5 | Moderately | T3N0M0 |
| | | | | (central type) | | | | |
| 15 | Female | 36 | Lung | Adenocarcinoma, | 1 M | 4 × 4 × 3.5 | Well | T2N0M0 |
| | | | | mucous | | | | |
| 16 | Male | 57 | Lung | Squamous cell | 2 M | 4 × 3.5 × 5 | Moderately | T2N0M0 |
| | | | | carcinoma | | | | |
| 17 | Male | 29 | Lung | Squamous cell | 3 M | f3.5 cm | Moderately | T2N0M0 |
| | | | | carcinoma | | | | |
| 18 | Male | 52 | Lung | Undifferentiated | 10 D | 4.5 × 4 × 3.5 | Poorly | T2N0M0 |
| | | | | small cell carcinoma | | | | |
| 19 | Male | 63 | Lung | Squamous cell | 1 M+ | 7.5 × 5 × 3 | Moderately | T3N0M0 |
| | | | | carcinoma | | | | |
| | | | | (Cornifying) | | | | |
| 20 | Male | 68 | Lung | Adenocarcinoma, | 1 M | f3 cm | Well | T2N1M0 |
| | | | | papillary (peripheral | | | | |
| | | | | type) | | | | |
| 21 | Male | 57 | Lung | Squamous cell | 5 M | 3 × 2 × 2 | Well | T2N0M0 |
| | | | | carcinoma | | | | |
| | | | | (central type, | | | | |
| | | | | cornifying) | | | | |
| 22 | Male | 52 | Lung | Squamous cell | 6 M | 5.5 × 3 × 2.5 | Moderately | T2N0M0 |
| | | | | carcinoma | | | | |
| 23 | Male | 46 | Lung | Squamous cell | 1 M+ | 6 × 5 × 4 | Well | T3N0M0 |
| | | | | carcinoma | | | | |
| | | | | (Cornifying) | | | | |
| 24 | Male | 58 | Lung | Squamous cell | 3 M | 3 × 2 | Moderately | T2N1M0 |
| | | | | carcinoma (central | | | | |
| | | | | type) | | | | |
| 25 | Male | 63 | Lung | Adenocarcinoma | 2 wk+ | 6.5 × 6 × 1 | Moderately | T3N0M0 |
| 26 | Female | 61 | Lung | Bronchio alveolar | 4 M+ | 3.5 × 3.5 × 2 | Well | T2N0M0 |
| | | | | carcinoma | | | | |
| 27 | Male | 40 | Lung | Squamous cell | 2 M | 6 × 4 × 2 | Well | T3N1M0 |
| | | | | carcinoma | | | | |
| 28 | Male | 64 | Lung | Squamous cell | 3 M | 8 × 7 × 9 | Moderately | T3N0M0 |
| | | | | carcinoma | | | | |
| 29 | Female | 44 | Lung | Adenosquamous | 6 M | 5 × 5 × 3.5 | Moderately | T2N1M0 |
| | | | | carcinoma | | | | |
| 30 | Male | 61 | Lung | Squamous cell | 5 M | 4 × 4 × 3.5 | Well | T2N0M0 |
| | | | | carcinoma | | | | |
| 31 | Female | 65 | Lung | Squamous cell | 3 M+ | f2.5 cm | Poorly | T1N0M0 |
| | | | | carcinoma | | | | |
| 32 | Female | 64 | Lung | Adenocarcinoma, | 1 M | 4 × 4.5 | Well | T2N0M0 |
| | | | | papillary (peripheral | | | | |
| | | | | type) | | | | |
| 33 | Male | 70 | Lung | Adenosquamous | N/A | f4 cm | Moderately | T2N1M0 |
| | | | | carcinoma | | | | |
| 34 | Male | 68 | Lung | Undifferentiated | 1 M | 3.7 × 3 × 2 | Poorly | T2N0M0 |
| | | | | small cell carcinoma | | | | |
| 35 | Male | 65 | Lung | Carcinoma | 1 M | 4 × 4 × 3 | Moderately | T2N0M0 |
| | | | | (peripheral type) | | | | |
| 36 | Female | 59 | Lung | Adenocarcinoma, | 1 M+ | 4 × 3.5 × 2.5 | Well | T2N0M0 |
| | | | | papillary | | | | |
| 37 | Male | 67 | Lung | Squamous cell | 2 M+ | f6 cm | Moderately | T2N0M0 |
| | | | | carcinoma | | | | |
| 38 | Male | 70 | Lung | Squamous cell | 1 M+ | f3.2 cm | Poorly | T2N0M0 |
| | | | | carcinoma | | | | |
| 39 | Female | 47 | Lung | Adenocarcinoma | 14 D+ | 4.5 × 4 × 3.5 | Moderately | T2N0M0 |
| 40 | Male | 71 | Lung | Squamous cell | 5 M | 4 × 2.5 | Moderately | T2N0M0 |
| | | | | carcinoma | | | | |
| 41 | Male | 65 | Lung | Squamous cell | 2 M+ | 15 × 10 × 12 | Moderately | T2N0M0 |
| | | | | carcinoma | | | | |
| 42 | Male | 68 | Lung | Adenocarcinoma, | 1 M | 8 × 4 × 4 | Moderately | T3N0M0 |
| | | | | squamous cell | | | | |
| | | | | carcinoma | | | | |
| 43 | Female | 39 | Lung | Adenocarcinoma | 1 M | f6 cm | Moderately | T2N1M0 |
| 44 | Male | 67 | Lung | Squamous cell | 18 M | 5 × 3.5 × 2.5 | Moderately | T2N1M0 |
| | | | | carcinoma | | | | |
| 45 | Female | 60 | Lung | Alveolus cell | 1 M | 3 × 2.5 × 2 | N/A | T2N0M0 |
| | | | | carcinoma | | | | |
| 46 | Female | 70 | Lung | Carcinoma | 1 M+ | f2 cm | Moderately | T1N0M0 |
| 47 | Male | 27 | Lung | Lung metastasis | 5 Y | 4 × 3.5 × 3 | Moderately | T2NxM1 |
| | | | | (sarcoma) | | | | |
| 48 | Male | 65 | Lung | Squamous cell | 2 M+ | 15 × 10 × 12 | Moderately | T3N0M0 |
| | | | | carcinoma | | | | |
| 49 | Female | 68 | Lung | Squamous cell | 4 M | 3 × 5 | Moderately | T2N0M0 |
| | | | | carcinoma | | | | |
| 50 | Female | 58 | Lung | Adenocarcinoma | 3 M | 3.7 × 3 × 2 | Moderately | T2N1M0 |
| 51 | Male | 68 | Lung | Squamous cell | 1 M+ | 5 × 5 × 3 | Well | T2N0M0 |
| | | | | carcinoma | | | | |
| 52 | Male | 48 | Lung | Squamous cell | 5 M | 7 × 6 × 4 | Moderately | T3N0M0 |
| | | | | carcinoma | | | | |
| 53 | Male | 59 | Lung | Squamous cell | 2 Y | f2 cm | N/A | T1N0M0 |
| | | | | carcinoma | | | | |
| 54 | Male | 54 | Lung | Adenocarcinoma, | 2 M | 5.5 × 3 × 3 | Moderately | T2N1M0 |
| | | | | cyst | | | | |
| 55 | Male | 45 | Lung | Squamous cell | 5 D | 6.5 × 4.5 × 3 | Moderately | T3N0M0 |
| | | | | carcinoma | | | | |
| 56 | Male | 69 | Lung | Squamous cell | 40 D | 5 × 4 × 4 | Poorly | T2N1M0 |
| | | | | carcinoma | | | | |
| 57 | Female | 78 | Lung | Alveolus cell | 2 M+ | 2.4 × 2 × 1.7 | Moderately | T1N0M0 |
| | | | | carcinoma | | | | |
| 58 | Male | 60 | Lung | Adenocarcinoma | 1 M+ | 2 × 2 × 1 | Moderately | T1N0M0 |
| 59 | Female | 54 | Lung | Alveolus cell | 1 M+ | 4 × 2.5 × 2 | Moderately | T2N1M0 |
| | | | | carcinoma | | | | |
| 60 | Male | 78 | Lung | Alveolus cell | 6 M+ | 1.5 × 0.5 × 0.3 | Moderately | T1N0M0 |
| | | | | carcinoma | | | | |
| 61 | Male | 70 | Lung | Alveolus cell | 1 wk | 2 × 1.7 × 0.8 | Well | T1N0M0 |
| | | | | carcinoma | | | | |
| 62 | Female | 45 | Lung | Bronchio alveolar | 1 M+ | f6 cm | Moderately | T2N0M0 |
| | | | | carcinoma | | | | |

In addition, microarray expression analysis data of a large number of clinical samples derived from Japanese subjects was examined and it was found that JARID1B expression was also significantly up-regulated in acute myelogenous leukemia, breast cancer, chronic myelogenous leukemia, cervical cancer and renal cell carcinoma (RCC), prostate cancer compared with corresponding non-neoplastic tissues, indicating its possible involvement in many types of human cancer (FIG. 5 and Table 6).

Interestingly, immunocytochemical staining using anti-JARID1B antibody indicated that its subcellular localization was altered after synchronizing the cells using aphidicolin. In G0/G1 phase and early S phase, 4 hour after the cell cycle release, the protein was located mainly in the nucleus (FIG. 6 upper). Later, the proteins were localized in both the nucleus and cytoplasm in S and G₂/M phases 12 hour after the cell cycle release (FIG. 6 lower). Therefore, the subcellular localization of JARID1B changes in response to the cell cycle status and this enzyme might potentially have a physiological role in the cytoplasm as well as in the nucleus.

TABLE 6
Expression of JARID1B in cancer tissues
analyzed by cDNA microarray*

| | | Ratio (Tumor/Normal) | | | |
|---|---|---|---|---|---|
| | Case | | | | >10 |
| Tissue type | (n) | >2 fold | >3 fold | >5 fold | fold |
| Acute myelogenous | 56 | 43 | 39 | 22 | 0 |
| leukemia (AML) | | (76.8%) | (69.6%) | (39.3%) | (0%) |
| Chronic myelogenous | 74 | 45 | 24 | 6 | 2 |
| leukemia (CML) | | (60.8%) | (32.4%) | (8.1%) | (2.7%) |
| Cervical cancer | 19 | 16 | 13 | 8 | 1 |
| | | (84.2%) | (68.4%) | (42.1%) | (5.3%) |
| Non-small cell | 37 | 28 | 18 | 7 | 0 |
| lung cancer | | (75.7%) | (48.6%) | (18.9%) | (0%) |
| Small cell lung cancer | 15 | 11 | 7 | 6 | 0 |
| | | (73.3%) | (46.7%) | (40%) | (0%) |
| Breast cancer | 80 | 33 | 11 | 3 | 0 |
| | | (41.3%) | (13.8%) | (3.8%) | (0%) |
| Bladder cancer | 34 | 21 | 6 | 3 | 0 |
| | | (61.28%) | (17.6%) | (8.8%) | (0%) |
| Renal cell carcinoma | 24 | 10 | 1 | 0 | 0 |
| | | (41.7%) | (4.2%) | (0%) | (0%) |

*We compared the signal intensity of JARID1B between tumor tissues and corresponding non-neoplastic tissues derived from the same patient.

### Example 3

JARID1B Affects Growth Regulation of Cancer Cells.

To investigate the role of JARID1B in human carcinogenesis, siRNA oligonucleotide duplexes, to specifically suppress the expression of JARID1B (siJARID1B#1, sequence aforementioned), together with two negative controls (siEGFP and siFFLuc) were prepared. Expression of JARID1B was tested in 17 untransfected bladder or lung cancer cell lines (FIG. 7A). Then, siRNAs were transfected into SW780 and A549 cells, where JARID1B was moderately expressed. Expression of JARID1B in cells containing siJARID1B#1 was significantly suppressed in both SW780 and A549, compared with that of controls (FIG. 7B). Cell growth assay was examined after treatment with siRNAs, in three bladder cancer cell lines (RT4, SW780, UMUC3), two non-small lung cancer cell lines (LC319, RERF-LC-A1) and one small lung cancer cell line (SBC5). In all cases, the growth of cancer cells was significantly suppressed after treatment with siJARID1B#1 (FIG. 7C).

To assess the mechanism of growth suppression induced by JARID1B-knockdown in cancer cells, the cell cycle status of cancer cells after treatment with siRNAs was analyzed using flow cytometry (FIG. 7D). The number of cells in sub-G1 phase notably increased after treatment with siJARID1B#1. This suggests the induction of apoptosis in response to suppression of JARID1B. A detailed analysis of siRNA transfected cells shows the proportion of cancer cells in sub-G1 phase was significantly higher in cells treated with siJARID1B#1, than for control-siRNA treated cancer cells (P=0.0055 [siEGFP, siJARID1B#1] and P=0.0042 [siFFLuc, siJARID1B#1], respectively, FIG. 7E). In addition, the proportion of siJARID1B treated cells in the later S and G2/M phases was significantly lower than control siRNA treated cells. Therefore, JARID1B can play a crucial role in the proliferation of cancer cells, and apoptosis may be induced by the knockdown of JARID1B.

Further, a knockdown experiment was performed using two independent siRNAs against JARID1B (siJARID1B#1 and #2) and two control siRNAs (siEGFP and siNC). These siRNAs were transfected into SW780, A549 and SBC5 cells in which JARID1B was highly expressed (FIG. 7A). Expression levels of JARID1B in the cells transfected with two independent siRNAs targeting JARID1B were significantly suppressed, compared to those transfected with control siRNAs (FIG. 7F). Using the same siRNAs, cell growth assays were performed and significant growth suppression by two independent siRNAs targeting JARID1B was observed in two bladder cancer cell lines (SW780 and RT4) and three lung cancer cell lines (A549, LC319 and SBC5) while no effect was observed when used control siRNAs (FIG. 7G).

### Example 4

Identification of the Downstream Genes by Microarray Expression Analysis.

Microarray expression analysis was performed to identify downstream genes of JARID1B and signal pathways induced by JARID1B. Total RNA was isolated from SW780 and A549 cells 24 hours after treatment with siJARID1B#1 and siJARID1B#2, and the expression profile of these cells was analyzed using Affymetrix's HG-U133 Plus 2.0 Array in comparison with those treated with control siRNAs (siEGFP and siFFLuc). As a result, a set of genes significantly up/down-regulated was identified. Further, a signal pathway analysis was performed, referring to the Gene Ontology database (Table 7), and found that JARID1B could be closely linked with the process of cell cycle regulation. Interestingly, significant down-regulation of E2F1 and E2F2 was observed by treatment with siJARID1B (P<0.0001 for both) (FIG. 8). Therefore, the functional relationship between JARID1B expression and the E2F/RB pathway, a key regulator pathway of the cell cycle, was analyzed.

TABLE 7
Gene Ontology pathway analysis based on the Affymetrix's microarray data

| Entry ID | Name | Definition | P |
|---|---|---|---|
| GO: 0007049 | Cell cycle | The progression of biochemical and | 1.15 × 10⁻⁹ |
| | | morphological phases and events that | |
| | | occur in a cell during successive cell | |
| | | replication or nuclear replication events. | |
| GO: 0022403 | Cell cycle phase | A cell cycle process comprising the steps | 2.12 × 10⁻⁸ |
| | | by which a cell progresses through one of | |
| | | the biochemical and morphological | |
| | | phases and events that occur during | |
| | | successive cell replication or nuclear | |
| | | replication events. | |
| GO: 0022402 | Cell cycle process | A cellular process that is involved in the | 8.26 × 10⁻⁸ |
| | | progression of biochemical and | |
| | | morphological phases and events that | |
| | | occur in a cell during successive cell | |
| | | replication or nuclear replication events. | |
| GO: 0000278 | Mitotic cell cycle | Progression through the phases of the | 2.80 × 10⁻⁷ |
| | | mitotic cell cycle, the most common | |
| | | eukaryotic cell cycle, which canonically | |
| | | comprises four successive phases called | |
| | | G1, S, G2, and M and includes replication | |
| | | of the genome and the subsequent | |
| | | segregation of chromosomes into | |
| | | daughter cells. In some variant cell cycles | |
| | | nuclear replication or nuclear division | |
| | | may not be followed by cell division, or | |
| | | G1 and G2 phases may be absent. | |
| GO: 0000279 | M phase | Progression through M phase, the part of | 1.45 × 10⁻⁶ |
| | | the cell cycle comprising nuclear | |
| | | division. | |
| GO: 0007067 | Mitosis | Progression through mitosis, the division | 2.41 × 10⁻⁶ |
| | | of the eukaryotic cell nucleus to produce | |
| | | two daughter nuclei that, usually, contain | |
| | | the identical chromosome complement to | |
| | | their mother. | |
| GO: 0000087 | M-phase of | Progression through M phase, the part of | 2.96 × 10⁻⁶ |
| | mitotic cell cycle | the mitotic cell cycle during which | |
| | | mitosis takes place. | |
| GO: 0043283 | Biopolymer | The chemical reactions and pathways | 1.91 × 10⁻⁵ |
| | metabolic process | involving biopolymers, long, repeating | |
| | | chains of monomers found in nature, such | |
| | | as polysaccharides and proteins. | |
| GO: 0044237 | Cellular | The chemical reactions and pathways by | 5.12 × 10⁻⁵ |
| | metabolic process | which individual cells transform chemical | |
| | | substances. | |
| GO: 0044238 | Primary | The chemical reactions and pathways | 5.44 × 10⁻⁵ |
| | metabolic process | involving those compounds which are | |
| | | formed as a part of the normal anabolic | |
| | | and catabolic processes. These processes | |
| | | take place in most, if not all, cells of the | |
| | | organism. | |
| GO: 0043170 | Macromolecule | The chemical reactions and pathways | 5.76 × 10⁻⁵ |
| | metabolic process | involving those compounds which are | |
| | | formed as a part of the normal anabolic | |
| | | and catabolic processes. These processes | |
| | | take place in most, if not all, cells of the | |
| | | organism. | |
| GO: 0000084 | S phase of mitotic | Progression through S phase, the part of | 1.38 × 10⁻⁴ |
| | cell cycle | the mitotic cell cycle during which DNA | |
| | | synthesis takes place. | |
| GO: 0051320 | S phase | Progression through S phase, the part of | 1.38 × 10⁻⁴ |
| | | the mitotic cell cycle during which DNA | |
| | | synthesis takes place. | |
| GO: 0051320 | Cell division | The process resulting in the physical | 1.47 × 10⁻⁴ |
| | | partitioning and separation of a cell into | |
| | | daughter cells. | |
| GO: 0006996 | Organelle | A process that is carried out at the cellular | 1.97 × 10⁻⁴ |
| | organization and | level which results in the formation, | |
| | biogenesis | arrangement of constituent parts, or | |
| | | disassembly of an organelle within a cell. | |
| | | An organelle is an organized structure of | |
| | | distinctive morphology and function. | |
| | | Includes the nucleus, mitochondria, | |
| | | plastids, vacuoles, vesicles, ribosomes | |
| | | and the cytoskeleton. Excludes the | |
| | | plasma membrane. | |
| GO: 0006260 | DNA | The process whereby new strands of | 2.38 × 10⁻⁴ |
| | | DNA are synthesized. The template for | |
| | | replication can either be an existing DNA | |
| | | molecule or RNA. | |

The down-regulation of E2F1 and E2F2 expression was observed in three different cancer cell lines, SW780, SBC5 and A549 treated with siRNAs, by quantitative real-time PCR (FIG. 9A, 9B). Moreover, higher expression levels of both E2F1 and E2F2 were found in clinical tumor tissues where JARID1B was over-expressed, than in non-neoplastic tissues (P=0.0009 and P=0.0002, respectively). The data indicates that both E2F1 and E2F2 could be highly expressed in tumor tissues correlating with elevated expression of JARID1B (Spearman's rank correlation coefficient: r=0.666 (E2F1) and r=0.756 (E2F2), respectively) (FIG. 9C).

To validate the transcriptional regulation of E2F by JARID1B in more detail, luciferase reporter assays were performed using an E2F-responsive luciferase construct (FIG. 9D). The construct was transformed into cancer cell lines after treatment with control (siEGFP) or siJARID1B. The E2F-driven transcriptional activity was significantly suppressed after treatment with siJARID1B#1 in both A549 and SBC5 cells. Furthermore, suppression of both E2F1 and E2F2 expressions was confirmed in A549 and SBC5 cells at the protein level after treatment with two independent siRNAs targeting JARID1B (FIG. 9E). These results reveal that the transcriptional activity regulated by E2F transcription factors can be suppressed after knockdown of JARID1B, and this disruption of this pathway may be responsible for the cell cycle alterations which we have observed.

These results indicate that JARID1B may work as one of the key factors of cell cycle regulation through E2F/RB pathway in human carcinogenesis.

Discussion

Histone modifications of chromatin, including methylation, acetylation, phosphorylation and ubiquitination, play a critical role in creating transcriptional activation and repression patterns, through the regulation of chromatin structure. JARID1B belongs to the lysine demethylase family, which specifically removes the methyl group of histone H3 lysine 4 (Yamane K et al., Mol Cell 2007, 25:801-812). In the present invention, significant up-regulation of JARID1B in bladder and lung cancers was demonstrated as well as various other cancer types, using quantitative RT-PCR, immunohistochemistry, and microarray-based gene expression profiles.

No significant JARID1B staining in vital organs was observed by immunohistochemical analysis (FIG. 1E, 3D, 10). Therefore, aberrant over-expression of JARID1B in any tumor, compared to corresponding non-neoplastic tissues, make it an ideal molecular target for cancer detection and as a therapeutic target. Already, synthetic inhibitors of classical HDACs have been widely used as tools in epigenetic studies, and many have shown growth-suppressive effects in cancer cells in vitro and have been used in early phase clinical trials(Jones P A et al. Cell 2007, 128:683-692; Paris M et al. J Med Chem 2008, 51:1505-1529). In addition, some histone methyltransferase and demethylase inhibitors have recently been reported (Greiner D et al. Nat Chem Biol 2005, 1:143-145; Huang Yet al. Proc Natl Acad Sci USA 2007, 104:8023-8028; Kubicek S et al. Mol Cell 2007, 25:473-481).

It was demonstrated that E2F1 and E2F2 are downstream modulators regulated by JARID1B. A luciferase reporter assay, combined with siRNA treatment, yielded indirect evidence supporting a molecular interaction between JARID1B and E2F elements. The E2F transcription factors are downstream effectors of the retinoblastoma (RB) protein pathway and are involved in many aspects of fundamental cell cycle control (Botz J et al. Mol Cell Biol 1996, 16:3401-3409; DeGregori J et al. Genes Dev 1995, 9:2873-2887; Johnson D G et al: Nature 1993, 365:349-352; Muller H et al. Biochim Biophys Acta 2000, 1470:M1-12). Binding sites for E2F factors have been identified in a large number of genes that control cell cycle and DNA synthesis, including cdk2 and 4, cyclin A, D and E, DNA polymerase, ribonucleotide reductase, UHRF1 and PCNA (Unoki M et al. Oncogene 2004, 23:7601-7610; Yamasaki L. Results Probl Cell Differ 1998, 22:199-227). Importantly, mutations in the RB-E2F cascade are found in a wide range of tumor types (Dimova D K et al. Oncogene 2005, 24:2810-2826; Nevins J R. Hum Mol Genet 2001, 10:699-703). Most of these alterations affect RB or upstream regulators of E2F transcriptional factors, and there is growing evidence that dysregulation of the E2F family itself is crucially involved in carcinogenesis. Indeed, in ovarian cancer, the proliferation-promoting E2F1 and E2F2 transcription factors were over-expressed, compared with healthy control tissues (Reimer D et al. Clin Cancer Res 2007, 13:144-151). Their dysregulation has been proposed as a prognostic indicator for various tumors (Ebihara Y et al. Dis Esophagus 2004, 17:150-154; Foster CS et al. Oncogene 2004, 23:5871-5879; Gorgoulis V G et al. J Pathol 2002, 198:142-156; Mega Set al. Dis Esophagus 2005, 18:109-113; Oeggerli M et al. Oncogene 2004, 23:5616-5623). Over-expression of a proliferation-promoting E2F transcription factor is argued to contribute a significant growth advantage to tumors especially those with poor prognosis. In the present study, we demonstrated significantly higher expression of both E2F1 and E2F2 in bladder tumor tissues than in non-neoplastic tissues, which is probably due to aberrant transcriptional regulation of JARID1B. Detailed pathway analysis on the basis of Gene Ontology revealed the involvement of JARID1B in several cell cycle processes (Table 7).

According to the microarray data, a number of other genes could be up-regulated by JARID1B. One of JARID1B's main functions was considered to be transcriptional repression through its demethylase activity because methylation of H3K4 is a marker for euchromatin (Yamane K et al. Mol Cell 2007, 25:801-812). From the microarray data, three possible mechanisms can be proposed whereby JARID1B can activate the transcription of its downstream genes. (i) Transcription is indirectly activated through transcription factors that are directly regulated by JARID1B; (ii) JARID1B transactivates expression of downstream candidates through protein-protein interaction. For example, JARID1B associates with the androgen receptor and enhances its transcriptional activity (Xiang Y et al. Proc Natl Acad Sci USA 2007, 104:19226-19231). JARID1B might both up- and down-regulate gene expressions, depending on its binding partners. (iii) JARID1B demethylates unknown substrates. Similarly, LSD1 was first reported to be a H3K4 specific demethylase (Shi Y et al. Cell 2004, 119:941-953), and later found to demethylate histone H3 lysine 9 and p53 (Huang J et al. Nature 2007, 449:105-108; Metzger E et al. Nature 2005, 437:436-439). Interestingly, JARID1B was found to be localized in the cytoplasm at some cell-cycle phases (FIG. 11,12), raising the possibility that it might demethylate unknown substrates in the cytoplasm and then affect cell cycle progression. The results obtained using several cancer cell lines strongly support the involvement of JARID1B in the growth of cancer cells.

### INDUSTRIAL APPLICABILITY

As demonstrated herein, cell growth is suppressed by the double-stranded molecule that specifically targets JARID1B gene. Thus, the novel double-stranded molecule are useful as anti-cancer pharmaceuticals. For example, agents that block the expression of JARID1B protein and/or prevent its activity have therapeutic utility as anti-cancer agents, particularly anti-cancer agents for the treatment of acute myelogenous leukemia, bladder cancer, breast cancer, chronic myelogenous leukemia, cervical cancer, lung cancer, prostate cancer and renal cell carcinoma, more particularly for the treatment of bladder cancer and lung cancer.

The expression of JARID1B is markedly elevated in cancer, as compared to normal organs. Accordingly, JARID1B genes or proteins can be conveniently used as diagnostic markers of cancer.

Furthermore, JARID1B polypeptide is a useful target for the development of anti-cancer pharmaceuticals. For example, agents that bind JARID1B polypeptide or block the expression of JARID1B gene or prevent its activity, may find therapeutic utility as anti-cancer agents, particularly anti-cancer agents for the treatment of acute myelogenous leukemia, bladder cancer, breast cancer, chronic myelogenous leukemia, cervical cancer, lung cancer, prostate cancer and renal cell carcinoma.

All publications, databases, sequences, patents, and patent applications cited herein are hereby incorporated by reference.

While the invention has been described in detail and with reference to specific embodiments thereof, it will be apparent to one skilled in the art that various changes and modifications can be made therein without departing from the spirit and scope of the invention, the metes and bounds of which are set by the appended claims.

1. A method for detecting or diagnosing cancer, said method comprising the steps of:
(a) determining the expression level of jumonji, AT rich interactive domain 1B (JARID1B) gene in a subject-derived biological sample by any one method selected from the group consisting of:
(i) detecting an mRNA of the JARID1B gene;
(ii) detecting a JARID1B protein; and
(iii) detecting the biological activity of the JARID1B protein; and
(b) relating an increase in the expression level determined in step (a) as compared to a normal control level of the gene to the presence of cancer.

2. The method of claim 1, wherein the cancer is selected from the group consisting of acute myelogenous leukemia, bladder cancer, chronic myelogenous leukemia, cervical cancer, lung cancer and renal cell carcinoma.

3. The method of claim 1, wherein the subject-derived biological sample comprises a biopsy sample, sputum, blood, pleural effusion or urine.

4. A kit for diagnosing cancer, wherein the kit comprises a reagent selected from the group consisting of:
(a) a reagent for detecting an mRNA of a JARID1B gene;
(b) a reagent for detecting a JARID1B protein; and
(c) a reagent for detecting the biological activity of the JARID1B protein.

5. The kit of claim 4, wherein the cancer is selected from the group consisting of acute myelogenous leukemia, bladder cancer, chronic myelogenous leukemia, cervical cancer, lung cancer and renal cell carcinoma.

6. An isolated double-stranded molecule, wherein said molecule, when introduced into a cell expressing the JARID1B gene, inhibits the expression of said gene, wherein said molecule comprises a sense strand and an antisense strand complementary thereto, wherein said strands hybridize to each other to form a double-stranded molecule, wherein said sense strand comprises a nucleic acid sequence corresponding to SEQ ID NO: 21 or 30, wherein said molecule has a length of between about 19 and about 25 nucleotides.

7. The double-stranded molecule of claim 6, which has one or two 3′ overhangs consisting of 2 to 10 nucleotides at either or both of the sense-strand and antisense-strand 3′ termini.

8. The double-stranded molecule of claim 6, which consists of a single polynucleotide comprising both the sense and antisense strands linked by an intervening single-stranded nucleic acid sequence.

9. The double-stranded molecule of claim 8, which has the general formula 5′-[A]-[B]-[A′]-3′, wherein [A] is the sense strand, [B] is the intervening single-stranded nucleic acid sequence consisting of 3 to 23 nucleotides, and [A′] is the antisense strand comprising a sequence complementary to [A].

10. A vector encoding the double-stranded molecule of claim 6.

11. Vectors comprising each of a combination of a polynucleotide comprising a sense strand nucleic acid and an antisense strand nucleic acid, wherein said sense strand nucleic acid comprises the nucleotide sequence of SEQ ID NO: 21 or 30 and said antisense strand nucleic acid consists of a sequence complementary to the sense strand, wherein the transcripts of said sense strand and said antisense strand hybridize to each other to form a double-stranded molecule, and wherein said vectors, when introduced into a cell expressing a JARID1B gene, inhibit cell proliferation.

12. A method for treating or preventing cancer, which comprises the step of administering to a subject an isolated double-stranded molecule or a vector encoding the double-stranded molecule, wherein said molecule, when introduced into a cell expressing the JARID1B gene, inhibits the expression of said gene, wherein said molecule comprises a sense strand and an antisense strand complementary thereto, wherein said strands hybridize to each other to form a double-stranded molecule, wherein said sense strand comprises a nucleic acid sequence corresponding to the nucleic acid sequence of JARID1B gene or a fragment thereof, and wherein said molecule has a length of between about 19 and about 25 nucleotides.

13. The method of claim 12, wherein said double-stranded molecule is a double-stranded molecule, wherein said molecule, when introduced into a cell expressing the JARID1B gene, inhibits the expression of said gene, wherein said molecule comprises a sense strand and an antisense strand complementary thereto, wherein said strands hybridize to each other to form a double-stranded molecule, wherein said sense strand comprises a nucleic acid sequence corresponding to SEQ ID NO: 21 or 30, wherein said molecule has a length of between about 19 and about 25 nucleotides.

14. The method of claim 12, wherein said cancer is selected from the group consisting of acute myelogenous leukemia, bladder cancer, breast cancer, chronic myelogenous leukemia, cervical cancer, lung cancer, prostate cancer and renal cell carcinoma.

15. A composition for treating or preventing cancer, which comprises an isolated double-stranded molecule or a vector encoding the double-stranded molecule, wherein said molecule, when introduced into a cell expressing the JARID1B gene, inhibits the expression of said gene, wherein said molecule comprises a sense strand and an antisense strand complementary thereto, wherein said strands hybridize to each other to form a double-stranded molecule, wherein said sense strand comprises a nucleic acid sequence corresponding to the nucleic acid sequence of JARID1B gene or a fragment thereof, and wherein said molecule has a length of between about 19 and about 25 nucleotides.

16. The composition of claim 15, wherein said double-stranded molecule is a double-stranded molecule, wherein said molecule, when introduced into a cell expressing the JARID1B gene, inhibits the expression of said gene, wherein said molecule comprises a sense strand and an antisense strand complementary thereto, wherein said strands hybridize to each other to form a double-stranded molecule, wherein said sense strand comprises a nucleic acid sequence corresponding to SEQ ID NO:21 or 30, wherein said molecule has a length of between about 19 and about 25 nucleotides.

17. The composition of claim 15, wherein said cancer is selected from the group consisting of acute myelogenous leukemia, bladder cancer, breast cancer, chronic myelogenous leukemia, cervical cancer, lung cancer, prostate cancer and renal cell carcinoma.

18. A method of screening for a candidate agent for treating or preventing cancer or inhibiting cancer cell growth, said method comprising the steps of
(a) contacting a test agent with a JARID1B polypeptide;
(b) detecting the biological activity of the polypeptide or detecting the binding activity between the polypeptide and the test agent; and
(c) selecting a test agent that suppresses the biological activity of the polypeptide as compared to the biological activity detected in the absence of the test agent as a candidate agent or selecting a test agent that binds to the polypeptide as a candidate agent.

19. (canceled)

20. The method of claim 18, wherein the biological activity is selected from the group consisting of cell proliferation activity, anti-apoptosis activity, promoting activity for the expression of E2F 1 gene or E2F2 gene, and demethylation activity.

21. The method of claim 20, said method comprising the steps of:
(a) contacting a JARID1B polypeptide with a substrate to be demethylated in the presence of a test agent under a suitable condition for demethylation of the substrate;
(b) detecting the methylation level of the substrate; and
(c) selecting the test agent that increases the methylation level of the substrate as compared to the methylation level detected in the absence of the test agent as a candidate agent.

22. The method of claim 21, wherein the substrate is a methylated histone H3.

23. The method of claim 22, wherein the methylation level is detected at lysine 4 of histone H3.

24. The method of claim 23, said method comprising the steps of:
(a) contacting a test agent with a cell expressing the JARID1B gene;
(b) detecting the methylation level of the histone H3 in the cell; and
(c) selecting the test agent that increases the methylation level of the histone H3 as compared to the methylation level detected in the absence of the test agent as a candidate agent.

25. (canceled)

26. A method of screening for a candidate agent for treating or preventing cancer, said method comprising the steps of:
(a) contacting a test agent with a cell expressing the JARID1B gene or contacting a test agent with a cell into which a vector, comprising the transcriptional regulatory region of JARID1B and a reporter gene that is expressed under the control of the transcriptional regulatory region, has been introduced;
(b) detecting the expression level of the JARID1B gene or measuring the expression level or activity of said reporter gene; and
(c) selecting a test agent that reduces the expression level of the JARID1B gene as compared to the expression level of JARID1B detected in the absence of the test agent as a candidate agent or selecting a test agent that reduces the expression or activity level of said reporter gene as compared to the expression or activity level of said reporter gene in the absence of the test agent as a candidate agent.

27. (canceled)

28. The method of claims 18, wherein the cancer is selected from the group consisting of acute myelogenous leukemia, bladder cancer, breast cancer, chronic myelogenous leukemia, cervical cancer, lung cancer, prostate cancer and renal cell carcinoma.

29. The method of claim 26, wherein the cancer is selected from the group consisting of acute myelogenous leukemia, bladder cancer, breast cancer, chronic myelogenous leukemia, cervical cancer, lung cancer, prostate cancer and renal cell carcinoma.


SEQUENCE LISTING (38 sequences)

SEQ ID NO: 1 - DNA, 6393 bp, Homo sapiens
gtacaactcggacttgctgttgctcgagccgcgtctgcacgggtctcggaccgagcggagctcgcagcctcggtcccggagcccaccttcgcctcgcccttgcccagcctgcggtgatggaggcggccaccacactgcacccaggcccgcgcccggcgctgcccctcgggggcccgggcccgctgggcgagttcctgcctccacccgagtgcccggtcttcgaacccagctgggaagagttcgcggaccccttcgctttcatccacaagatccggcccatagccgagcagactggcatctgtaaggtgcggccgccgccggattggcagccaccatttgcatgtgatgttgataaacttcattttacgccacgtatccagagactgaatgaattggaggcccaaactcgtgtaaaattgaatttcttggaccagattgcaaagtactgggagttacagggaagtactctgaaaattccacatgtggagaggaagatcttggacttatttcagcttaataagttagttgcagaagaaggtggatttgcagttgtttgcaaggatagaaaatggaccaaaattgctaccaagatggggtttgctcctggcaaagcagtgggctcacatatcagagggcattatgaacgaattctcaacccctacaacttattcctgtccggagacagcctaaggtgtttgcagaagccaaacctgaccacagacactaaggacaaggagtacaaaccccatgatattccccagaggcagtctgtgcagccttcggaaacgtgccccccagcccgacgagcaaaacgcatgagagcagaggccatgaatattaaaatagaacccgaggagacaacggaagccagaactcataatctgagacgtcgaatgggttgtccaactccaaaatgtgaaaatgagaaagaaatgaagagtagcatcaagcaagaacctattgagaggaaagattatattgtagaaaatgagaaggaaaagcccaagagtcgatctaaaaaagccaccaatgctgtggacctgtatgtctgtcttttatgtggcagtggcaatgatgaagaccggctactgttgtgtgatggctgtgatgacagttaccataccttttgcttgatcccacctctccatgatgttcccaagggagactggaggtgtcctaagtgtttggctcaggaatgtagtaagccacaagaagcatttggctttgaacaagcagccagggactataccctccgtacttttggggaaatggcagatgcgttcaaatctgattacttcaacatgccagtccatatggtccccacagagcttgttgagaaagaattttggagactagtaagcactattgaggaggatgtcacagtggaatatggagctgacattgcctcaaaggaatttggcagtggctttcctgtccgagatgggaaaatcaaactctcacctgaggaagaggagtatcttgatagtggctggaatttgaacaacatgccagtgatggagcagtctgtccttgcacatattactgctgatatatgtggcatgaaacttccttggttgtatgtgggaatgtgcttttcttcattctgttggcacattgaagaccactggagctattcaattaactacttgcactggggtgagccaaaaacctggtatggagtcccagggtatgctgctgagcagctagaaaatgtaatgaagaaactagctccagaactctttgtgtcccagccggatctcctccatcagcttgtgaccatcatgaaccccaataccctgatgactcatgaagtgcctgtttaccgaactaatcagtgtgctggggagtttgtgattacatttccaagagcctaccacagtggttttaaccagggttttaattttgctgaggctgttaacttctgcactgttgattggctgccattaggccgacagtgtgtggagcattatcgcttgcttcatcgatattgtgtgttttcccacgatgagatgatctgcaagatggcttccaaggctgatgtattagatgttgtagtggcttcaactgttcagaaagacatggccattatgattgaggatgagaaagctttaagagaaactgtccgtaaattgggagtgattgattcggaaagaatggattttgagctgttgccagatgatgaacgtcagtgtgtaaaatgcaaaactacatgcttcatgtctgccatctcctgttcttgtaaacctggccttcttgtttgcctgcatcatgtaaaagaattgtgttcctgtcctccttacaaatataaattgcggtataggtacacgctggatgatctctaccctatgatgaatgcattgaagcttcgagcagaatcttacaacgaatgggccttgaatgtgaatgaagctttggaggcaaagatcaacaagaagaaaagccttgtcagcttcaaggctttaattgaagaatctgaaatgaagaaattcccagacaatgatcttttgcgacaccttcgcctagtcacacaggatgcagagaagtgtgcctctgttgcgcagcagttgcttaatggcaaaaggcaaactagatatcgatctggtggagggaaatcccaaaatcagttgacagtgaatgagctccggcagtttgtaacacagctgtatgctcttccatgtgtcctcagtcagacaccattactaaaggatctcttgaatcgtgtagaagattttcaacagcatagtcagaaactactctctgaggaaacgcctagtgctgcggagctgcaggacttgctagatgtcagctttgaatttgatgttgaacttccacagcttgctgagatgcgtatccgtttggaacaagcccgttggctagaagaggtgcagcaagcttgcctagaccccagctcccttactttagatgatatgagacgtctcatagacctaggggtagggctggccccgtattcagcagtggagaaagctatggcccggctgcaggaactgctcacagtgtcagagcactgggacgacaaagccaagagtctcctcaaggccaggccacgacattcattgaatagccttgctacggcagtaaaggaaatcgaagagatccctgcatatctgcccaatggtgcggctctgaaagactcagtgcagagagccagagactggcttcaggatgtagagggcctgcaggctggaggacgtgtgccagtgttagacacactcatagaacttgttacacgaggccgatctatccccgtacatctgaattctttgccaagactggaaaccctagtagctgaggttcaggcttggaaagaatgtgctgttaatacattcttgactgagaattctccatattctctcttagaggtgctgtgtcctcgatgtgatattggccttttgggattgaaaaggaagcagagaaagttaaaggagcccttgccaaatggaaagaaaaaaagcaccaaattagagagtctgagtgacctggagagagctttaactgaaagcaaggagactgcttcagctatggcaactcttggggaagctcgcctaagggaaatggaagccttgcagtctctcagactcgccaatgaagggaaattgctgtcgcctctccaagatgtggatataaaaatctgcctatgtcagaaggccccagctgcccctatgattcaatgtgaactctgcagggatgctttccacaccagttgtgtggcggtacccagtatttcacagggcctgcgaatctggctttgtccccattgtcggaggtcagagaaacctccattagagaaaattctgcccctgctcgcctcccttcagcgtatccgagttcgccttcctgagggagatgcacttcgatatatgattgaaagaaccgtgaactggcagcacagagcccagcaactgctttcgtcagggaatcttaaatttgtgcaagatcgagtgggctcaggactgttatatagcagatggcaagcctcagcaggacaggtgtcagacacaaacaaggtatctcaacctcctggcacaacatcattttctttgcctgatgactgggacaacagaacctcatatttgcactcccccttctcaactggacgaagttgtatccccctccatggtgttagtccagaagtgaatgaactattgatggaagcccagctgctccaggtatcccttcctgaaattcaggaactttaccagactttacttgcaaagccaagccctgctcagcagactgaccgaagctcaccagtgagacccagcagtgagaagaatgactgttgccgagggaagcgagatggaattaacagtcttgagagaaaactgaagagacgcctggaaagagagggcctctccagtgagcggtgggaacgagttaagaaaatgcggacccccaaaaagaagaaaatcaaactgagccaccccaaggacatgaacaatttcaagttagagagagagcgtagctatgaattagttcgttctgctgaaactcattccctgccctcagacacatcctattccgaacaggaagactctgaggatgaagatgccatctgcccagctgtgagctgcctgcagccagaaggagatgaggtggactgggtccagtgtgatggcagctgcaatcagtggtttcatcaggtctgtgttggtgtctccccagagatggcagagaaagaagactacatctgtgtgcgctgtactgtgaaggacgcaccaagccgaaagtaaaaacacaaaaacagatacccccctacttaatgtaattcaggactccaaccaagaggatttcttcaaatctcagcaaagctacaggactggtactcaagccagcctgtaaacggtgctatttctattccttatgggatcatttttccaggactctttgaagaaaagaaaaaacaactaaaaaaatttttgacactttttgtattttttccttaagagctatttgtggttgttgaggtttgaaaagctgactgttttttttgcaggggttcccaccaatttggaaggcattgaagcttgcaccttttcatgtacagcattaaaattttacctctctctgggatttaccagcttaagagtccaactcacttccagtgcccaaaagggcacccaccagaaattccagtaaatcctcatttgaggaagctctcccttgtttactctgttaccacattggggaaatttttaagtttttcactttgggagtttttgtttgtttcttcttttcctttatccacttttcttcttcctggtagactaggtttatttatctgagcaataacttctatgttggtttcagtggctggaattaaaacaaaacaaaacaaacttccaaacagtgtgttggtgctttagcgattgattgatgtacagaacacaaatgtctagtttctagtgtcactgatgaactagtgatgtagaaaagagacttctctgtaagtaattgccacagctgtattttggctttctccctgtcccttcctttctcccctattttttggtagcttgtatcaaatgttacagtttatattgtggaataaatccttcgtcctaacataacactaaatgctgattatttagagccattagagcacagcttcttctgcccctctactgttgcacagccagaaggggctgcttgcttttgcctctgcccaaccaggttgcagtagcagtggatgttagcctgcaaacaacattaggggattcttcttttgtgtcctgctctgtttggtgggccatatgcctacagccctcactaacaaagaacccctctgtcttcaaggactagaacctatctttaaagccgtgctcttttaaaataagctttctcagaatgttggcaaatcacttcaatcctcaaatcagtcttccttgtggaatgctgcctttattatatttgaattgacaggggaacttggtttagggttaagacgttggaaggaaatctaaggaaaattaatcctcacagaggtccgggtttagtatatgtcttgagaggagacttgtgaattcccagactctgcctcctggtttcctttctcatttctttctaactgtagctatcattactggctgtggatagcccatagctattttccttgcttttcttttttaaaggggtctgttctgcagtggagaagacattctggtgaccagacttttgcttaccttttcctattctgttgccaatttttgttttccccacattctatagccataaacctgaagatgagtaaaactggtgggtctttaataaaacaacaacaaaaacagcagtttgtgatatagcagaggtttaaatgtaccctccccttttatgcacttcaaataattaaatctctttaagaatgaaaaaaaaaaa

SEQ ID NO: 2 - protein, 1544 aa, Homo sapiens
MEAATTLHPGPRPALPLGGPGPLGEFLPPPECPVFEPSWEEFADPFAFIHKIRPIAEQTGICKVRPPPDWQPPFACDVDKLHFTPRIQRLNELEAQTRVKLNFLDQIAKYWELQGSTLKIPHVERKILDLFQLNKLVAEEGGFAVVCKDRKWTKIATKMGFAPGKAVGSHIRGHYERILNPYNLFLSGDSLRCLQKPNLTTDTKDKEYKPHDIPQRQSVQPSETCPPARRAKRMRAEAMNIKIEPEETTEARTHNLRRRMGCPTPKCENEKEMKSSIKQEPIERKDYIVENEKEKPKSRSKKATNAVDLYVCLLCGSGNDEDRLLLCDGCDDSYHTFCLIPPLHDVPKGDWRCPKCLAQECSKPQEAFGFEQAARDYTLRTFGEMADAFKSDYFNMPVHMVPTELVEKEFWRLVSTIEEDVTVEYGADIASKEFGSGFPVRDGKIKLSPEEEEYLDSGWNLNNMPVMEQSVLAHITADICGMKLPWLYVGMCFSSFCWHIEDHWSYSINYLHWGEPKTWYGVPGYAAEQLENVMKKLAPELFVSQPDLLHQLVTIMNPNTLMTHEVPVYRTNQCAGEFVITFPRAYHSGFNQGFNFAEAVNFCTVDWLPLGRQCVEHYRLLHRYCVFSHDEMICKMASKADVLDVVVASTVQKDMAIMIEDEKALRETVRKLGVIDSERMDFELLPDDERQCVKCKTTCFMSAISCSCKPGLLVCLHHVKELCSCPPYKYKLRYRYTLDDLYPMMNALKLRAESYNEWALNVNEALEAKINKKKSLVSFKALIEESEMKKFPDNDLLRHLRLVTQDAEKCASVAQQLLNGKRQTRYRSGGGKSQNQLTVNELRQFVTQLYALPCVLSQTPLLKDLLNRVEDFQQHSQKLLSEETPSAAELQDLLDVSFEFDVELPQLAEMRIRLEQARWLEEVQQACLDPSSLTLDDMRRLIDLGVGLAPYSAVEKAMARLQELLTVSEHWDDKAKSLLKARPRHSLNSLATAVKEIEEIPAYLPNGAALKDSVQRARDWLQDVEGLQAGGRVPVLDTLIELVTRGRSIPVHLNSLPRLETLVAEVQAWKECAVNTFLTENSPYSLLEVLCPRCDIGLLGLKRKQRKLKEPLPNGKKKSTKLESLSDLERALTESKETASAMATLGEARLREMEALQSLRLANEGKLLSPLQDVDIKICLCQKAPAAPMIQCELCRDAFHTSCVAVPSISQGLRIWLCPHCRRSEKPPLEKILPLLASLQRIRVRLPEGDALRYMIERTVNWQHRAQQLLSSGNLKFVQDRVGSGLLYSRWQASAGQVSDTNKVSQPPGTTSFSLPDDWDNRTSYLHSPFSTGRSCIPLHGVSPEVNELLMEAQLLQVSLPEIQELYQTLLAKPSPAQQTDRSSPVRPSSEKNDCCRGKRDGINSLERKLKRRLEREGLSSERWERVKKMRTPKKKKIKLSHPKDMNNFKLERERSYELVRSAETHSLPSDTSYSEQEDSEDEDAICPAVSCLQPEGDEVDWVQCDGSCNQWFHQVCVGVSPEMAEKEDYICVRCTVKDAPSRK

SEQ ID NO: 3 - DNA, 20 bp, Artificial
  An artificially synthesized primer sequence for RT-PCR
gcaaattccatggcaccgtc

SEQ ID NO: 4 - DNA, 19 bp, Artificial
  An artificially synthesized primer sequence for RT-PCR
tcgccccacttgattttgg

SEQ ID NO: 5 - DNA, 20 bp, Artificial
  An artificially synthesized primer sequence for RT-PCR
tgggaacaagagggcatctg

SEQ ID NO: 6 - DNA, 22 bp, Artificial
  An artificially synthesized primer sequence for RT-PCR
ccaccactgcatcaaattcatg

SEQ ID NO: 7 - DNA, 25 bp, Artificial
  An artificially synthesized primer sequence for RT-PCR
attgcctcaaaggaatttggcagtg

SEQ ID NO: 8 - DNA, 25 bp, Artificial
  An artificially synthesized primer sequence for RT-PCR
catcactggcatgttgttcaaattc

SEQ ID NO: 9 - DNA, 25 bp, Artificial
  An artificially synthesized primer sequence for RT-PCR
tgtcacagtggaatatggagctgac

SEQ ID NO: 10 - DNA, 25 bp, Artificial
  An artificially synthesized primer sequence for RT-PCR
gccactatcaagatactcctcttcc

SEQ ID NO: 11 - DNA, 22 bp, Artificial
  An artificially synthesized primer sequence for RT-PCR
gctggaccacctgatgaatatc

SEQ ID NO: 12 - DNA, 23 bp, Artificial
  An artificially synthesized primer sequence for RT-PCR
tctgcaatgctacgaaggtcctg

SEQ ID NO: 13 - DNA, 23 bp, Artificial
  An artificially synthesized primer sequence for RT-PCR
tggcaactttaaggagcagacag

SEQ ID NO: 14 - DNA, 22 bp, Artificial
  An artificially synthesized primer sequence for RT-PCR
gggcacaggtagacttcgatgg

SEQ ID NO: 15 - DNA, 21 bp, Artificial
  An artificially synthesized oligonucleotide for siRNA
gcagcacgacuucuucaagtt

SEQ ID NO: 16 - DNA, 21 bp, Artificial
  An artificially synthesized oligonucleotide for siRNA
cuugaagaagucgugcugctt

SEQ ID NO: 17 - DNA, 21 bp, Artificial
  An artificially synthesized oligonucleotide for siRNA
gugcgcugcuggugccaactt

SEQ ID NO: 18 - DNA, 21 bp, Artificial
  An artificially synthesized oligonucleotide for siRNA
guuggcaccagcagcgcactt

SEQ ID NO: 19 - DNA, 21 bp, Artificial
  An artificially synthesized oligonucleotide for siRNA
cagugaaugagcuccggcatt

SEQ ID NO: 20 - DNA, 21 bp, Artificial
  An artificially synthesized oligonucleotide for siRNA
ugccggagcucauucacugtt

SEQ ID NO: 21 - DNA, 19 bp, Artificial
  An artificially synthesized oligonucleotide for siRNA target sequence
cagtgaatgagctccggca

SEQ ID NO: 22 - DNA, 19 bp, Artificial
  An artificially synthesized oligonucleotide for siRNA
auccgcgcgauaguacgua

SEQ ID NO: 23 - DNA, 19 bp, Artificial
  An artificially synthesized oligonucleotide for siRNA
uacguacuaucgcgcggau

SEQ ID NO: 24 - DNA, 19 bp, Artificial
  An artificially synthesized oligonucleotide for siRNA
uuacgcguagcguaauacg

SEQ ID NO: 25 - DNA, 19 bp, Artificial
  An artificially synthesized oligonucleotide for siRNA
cguauuacgcuacgcguaa

SEQ ID NO: 26 - DNA, 19 bp, Artificial
  An artificially synthesized oligonucleotide for siRNA
uauucgcgcguauagcggu

SEQ ID NO: 27 - DNA, 19 bp, Artificial
  An artificially synthesized oligonucleotide for siRNA
accgcuauacgcgcgaaua

SEQ ID NO: 28 - DNA, 21 bp, Artificial
  An artificially synthesized oligonucleotide for siRNA
ggaauauggagcugacauutt

SEQ ID NO: 29 - DNA, 21 bp, Artificial
  An artificially synthesized oligonucleotide for siRNA
aaugucagcuccauauucctt

SEQ ID NO: 30 - DNA, 19 bp, Artificial
  An artificially synthesized oligonucleotide for siRNA target sequence
ggaatatggagctgacatt

SEQ ID NO: 31 - RNA, 19 nt, Artificial
  An artificially synthesized oligonucleotide for siRNA
cagugaaugagcuccggca

SEQ ID NO: 32 - RNA, 19 nt, Artificial
  An artificially synthesized oligonucleotide for siRNA
ugccggagcucauucacug

SEQ ID NO: 33 - RNA, 19 nt, Artificial
  An artificially synthesized oligonucleotide for siRNA
ggaauauggagcugacauu

SEQ ID NO: 34 - RNA, 19 nt, Artificial
  An artificially synthesized oligonucleotide for siRNA
aaugucagcuccauauucc

SEQ ID NO: 35 - DNA, 2722 bp, Homo sapiens
  CDS (141)..(1454)
ttggcgcgtaaaagtggccgggactttgcaggcagcggcggccgggggcggagcgggatcgagccctcgccgaggcctgccgccatgggcccgcgccgccgccgccgcctgtcacccgggccgcgcgggccgtgagcgtcatggccttggccggggcccctgcgggcggcccatgcgcgccggcgctggaggccctgctcggggccggcgcgctgcggctgctcgactcctcgcagatcgtcatcatctccgccgcgcaggacgccagcgccccgccggctcccaccggccccgcggcgcccgccgccggcccctgcgaccctgacctgctgctcttcgccacaccgcaggcgccccggcccacacccagtgcgccgcggcccgcgctcggccgcccgccggtgaagcggaggctggacctggaaactgaccatcagtacctggccgagagcagtgggccagctcggggcagaggccgccatccaggaaaaggtgtgaaatccccgggggagaagtcacgctatgagacctcactgaatctgaccaccaagcgcttcctggagctgctgagccactcggctgacggtgtcgtcgacctgaactgggctgccgaggtgctgaaggtgcagaagcggcgcatctatgacatcaccaacgtccttgagggcatccagctcattgccaagaagtccaagaaccacatccagtggctgggcagccacaccacagtgggcgtcggcggacggcttgaggggttgacccaggacctccgacagctgcaggagagcgagcagcagctggaccacctgatgaatatctgtactacgcagctgcgcctgctctccgaggacactgacagccagcgcctggcctacgtgacgtgtcaggaccttcgtagcattgcagaccctgcagagcagatggttatggtgatcaaagcccctcctgagacccagctccaagccgtggactcttcggagaactttcagatctcccttaagagcaaacaaggcccgatcgatgttttcctgtgccctgaggagaccgtaggtgggatcagccctgggaagaccccatcccaggaggtcacttctgaggaggagaacagggccactgactctgccaccatagtgtcaccaccaccatcatctcccccctcatccctcaccacagatcccagccagtctctactcagcctggagcaagaaccgctgttgtcccggatgggcagcctgcgggctcccgtggacgaggaccgcctgtccccgctggtggcggccgactcgctcctggagcatgtgcgggaggacttctccggcctcctccctgaggagttcatcagcctttccccaccccacgaggccctcgactaccacttcggcctcgaggagggcgagggcatcagagacctcttcgactgtgactttggggacctcacccccctggatttctgacagggcttggagggaccagggtttccagagatgctcaccttgtctctgcagccctggagccccctgtccctggccgtcctcccagcctgtttggaaacatttaatttatacccctctcctctgtctccagaagcttctagctctggggtctggctaccgctaggaggctgagcaagccaggaagggaaggagtctgtgtggtgtgtatgtgcatgcagcctacacccacacgtgtgtaccgggggtgaatgtgtgtgagcatgtgtgtgtgcatgtaccggggaatgaaggtgaacatacacctctgtgtgtgcactgcagacacgccccagtgtgtccacatgtgtgtgcatgagtccatgtgtgcgcgtgggggggctctaactgcactttcggcccttttgctctgggggtcccacaaggcccagggcagtgcctgctcccagaatctggtgctctgaccaggccaggtggggaggctttggctggctgggcgtgtaggacggtgagagcacttctgtcttaaaggttttttctgattgaagctttaatggagcgttatttatttatcgaggcctctttggtgagcctggggaatcagcaaaggggaggaggggtgtggggttgataccccaactccctctacccttgagcaagggcaggggtccctgagctgttcttctgccccatactgaaggaactgaggcctgggtgatttatttattgggaaagtgagggagggagacagactgactgacagccatgggtggtcagatggtggggtgggccctctccagggggccagttcagggccccagctgccccccaggatggatatgagatgggagaggtgagtgggggaccttcactgatgtgggcaggaggggtggtgaaggcctcccccagcccagaccctgtggtccctcctgcagtgtctgaagcgcctgcctccccactgctctgccccaccctccaatctgcactttgatttgcttcctaacagctctgttccctcctgctttggttttaataaatattttgatgacgtttgggccgggttttgggactctgttgggaacatttcggggcgggagaggccaaggttgctggggaaatgcccattctccacttcccttctccctgtccgtgcccgatttgatttgagcctcataactcgaagaaaggtcagcttcctcgctgttttggtcctaactcaaaagcagatccagtaaaggtttttgttgtaaaaaaaaaaaaaaaaaaaaaaa

SEQ ID NO: 36 - protein, 437 aa, Homo sapiens
MALAGAPAGGPCAPALEALLGAGALRLLDSSQIVIISAAQDASAPPAPTGPAAPAAGPCDPDLLLFATPQAPRPTPSAPRPALGRPPVKRRLDLETDHQYLAESSGPARGRGRHPGKGVKSPGEKSRYETSLNLTTKRFLELLSHSADGVVDLNWAAEVLKVQKRRIYDITNVLEGIQLIAKKSKNHIQWLGSHTTVGVGGRLEGLTQDLRQLQESEQQLDHLMNICTTQLRLLSEDTDSQRLAYVTCQDLRSIADPAEQMVMVIKAPPETQLQAVDSSENFQISLKSKQGPIDVFLCPEETVGGISPGKTPSQEVTSEEENRATDSATIVSPPPSSPPSSLTTDPSQSLLSLEQEPLLSRMGSLRAPVDEDRLSPLVAADSLLEHVREDFSGLLPEEFISLSPPHEALDYHFGLEEGEGIRDLFDCDFGDLTPLDF

SEQ ID NO: 37 - DNA, 5273 bp, Homo sapiens
  CDS (429)..(1742)
aaggactagagagcgagccgcaaggaagtcggtgcagtcgagacccccctccccatcccagcgcatcgcgtctccgccgagcttgagggcacgccggggacccctccccagagccggccggaccccaggtgccgaggccttggggagcgcggggcgtcccgggtcgcggtgccctcgggacgagacagcccctggcagtgccaccaccgcagccgccgggcgatctccaagcggcgatctctaagcgctgctctctgctcggccgcgggccaggaggggagggtccggccttgccccgcaggcgtccattggcggcttcccccggcctccgcgccatgccgcgggccgtgtgaaaggcggcagcaccggaacccgcaggtgtccgcgggcgcgccaagcccttttgggtagggggcgccttactcgctatgctgcaagggccccgggccttggcttcggccgctgggcagaccccgaaggtggtgcccgcgatgagccccacagagctgtggccatccggcctcagcagcccccagctctgcccagctactgctacctactacacaccgctgtacccgcagacggcgcctcccgcagcggcgccaggcacctgcctcgacgccactccccacggacccgagggccaagttgtgcgatgcctgccggcaggccggctgccggccaaaaggaagctggatctggaggggattgggaggcccgtcgtccctgagttcccaacccccaaggggaagtgcatcagagtggatggcctccccagccccaaaacccccaaatcccccggggagaagactcggtatgacacttcgctggggctgctcaccaagaagttcatttacctcctgagcgagtcagaggatggggtcctggacctgaactgggccgctgaggtgctggacgtgcagaagcggcgcatctatgacatcaccaacgtgctggaaggcatccagctcatccgcaagaaggccaagaacaacatccagtgggtaggcaggggaatgtttgaagaccccaccagacctgggaagcagcaacagctggggcaggagctgaaggagctgatgaacacggagcaggccttggaccagctcatccagagctgctctctgagcttcaagcacctgactgaggacaaggccaacaagaggctggcctatgtgacttaccaggatatccgtgctgttggcaactttaaggagcagacagtgattgccgtcaaggcccctccgcagacgagactggaagtgcccgacaggactgaggacaacctgcagatatatctcaagagcacccaagggcccatcgaagtctacctgtgcccagaggaggtgcaggagccggacagtccttccgaggagcctctcccctctacctccaccctctgccccagccctgactctgcccagcccagcagcagcaccgaccctagcatcatggagcccacagcatcctcagtgccagcaccagcgccaaccccccagcaggccccaccgcctccatccctggtccccttggaggctactgacagcctgctggagctgccgcacccactcctgcagcagactgaggaccagttcctgtccccgaccctggcgtgcagctcccctctgatcagcttctccccatccttggaccaggacgactacctgtggggcttggaggcgggtgagggcatcagcgatctcttcgactcctacgaccttggggacctgttgattaattgagtggccctgcctgcccccagcagcctgcccccgactctacctcctcacagacaggctgacagcccctctgcctgcacagggacattggacactaggtgctgccctcagggcatggggtctcctcgcctttcctgccccagccggcagaagctgtgtggggagatatgaatggtacgggtgaggagtggataaggggtggtcctcaccttcctaatggaagctgggcctagggaggcccatccagtcttctgacttctgacctctcacaagaaggctgcaggtgaggtggccaagtccagggaaaggccctgctacctccttttgaggggtaattaggaccctcgacgtaccaagaagcacataatgcctttgtatttatttcaggttgagttgtttgtttgtcctccctgagttttagcagggaggttgttctagtttttagtgagacctctgcagacaggcccatcactgtccatgttccagggcaggtctgggtttccaagggaggggcccaggctacatccttggtttccccactgtggtgggggctgggactctgaggggctgtccagtctgctagaatgctaattgcacttaggcctcatggttctagtaaacggcagctgtgggcccttttgcctcttcccctgttcttggcctcacatctccagctgagctgccggtcttggcttcctggtcgcctctgtcccagagatggtcccagggagccatcctagggcaggtagcactgaggctcctgtggaaacaggagccacctgctcaggagacccctttcctgaggaagtccttacctctccccttgagatgtaaaaatggtccagcagagacaagctcccgtggaaaacagacaggagcatgggggcagctgtcatggctgtggcgggcacttttcctcagagtttctgccttgcgctggtccaggagccattttgcaccaaggacttggtaggcagaggcagccccactgtaaagaagggtcagattaaaacaaaaaactgccaaaagcatcccctctgccccccatgtggcactggcatcattctctgcttccctgggaggaattttttcaccatgttattgaaggggatggttcattaaggactccacccctcagagctcactcagaccccaaggacagaggtgactggggcttggtgacttgttcactccttttttcccaggtatactgaaggggtgacagagagaggtcttcatggcagaccaggccttcacagctaatggggagaggaactcatgttacctctgcaggcctggggtcctgagggggtcttttggcttcagcctgttcccccagaggcttgatcatcccacattgtcccttcagctcagctgctcttctcccccacccaccctgggatgtgggtgctctgggctgaaccaaggctatgacttctggagagaggctcaggggttggtctgagaggcctgccatccacccctcagggagctaggttttctcagaggctcagctggacagcactttttagaaaagtttgtagcattaagctggtttaaaatatgaagttggttttgttggatggctcctgagctgactgactgatgtctgaagtttgagacgagggattatttcagggtggggcccaatgtgatctaatgcccagctggggacaattgtgcctcatcatttgctcaaattcctgggcccccaagttagccccctcccaggagtggtcagcgggtcacagctgcccccactctataagcagggctaattgtgtaccctttgcagaaatgcttttggtctcctacccaaatactcacaagggtcttatcagacgcccgtcttaaagtccagcatgctcagggaccctgtgtaggatctcgtttgtggtgagtgggctgctctgaggtctccactgggctgccatttagccatgtgccatctctgaagtcagaggtgtttgactcccattccttgggctctggagctttccccaagaattacatcagagaaaaggaagaaggggcctgcaggacccattgggaatgagtttaatactgaagtctggaatgtaagctcatgccctagaggcctctccatatggctggtcaggggagctgccttcaggcttgtgccccgtgtgctcagcagctgcctctgtccccctctactgtccctttcacaccttgcctggccaaggggctagacctcccaggctaagcctcagattcagtgcaggacacaagctcatgcccccgtcttgccagtgacacttgaagcctcccgacttccacagagtgcttcaggacacattttgagtggtattttcttttctttttttcttctttttttttttttttgagatggagtctcgctctgttgcccaggctggagtgcagtggcctgatctcggctcactgcaacctctgcctcccaggttcaagcgattcttctgcctcagcctccagagtagctgggactatagacatgcaccaccacgcccggctaattttgtatttttggtcgagacggggttttgccatgttagtcaggctggtcttgaactcctgacctcaagtgatccaccacctcggcctcccaaagtgttgagatgacaggcacgagccaccaggcccagcctgagtggtattttctttagggaccaggtagactttaaaacgagggtaagagaaaagccagtgtctttctgaggtaaataatttctgccaggaaacttcccagccccaccagcagcccccctaaaaaaatcactcgtgtccccagggacttctaaagcttggggctccaggaaatcatccagtagagttggagattcagagatttcttgaagccagggacatgctcctaactcctttcccattaaaggtgttagaatagaccagagggtgtcccttttccacagtaatgggatcggctggtgtgccttcagggaggaagagggaggtggtcaagcttgaaaaactggctttaggatggttctgactttgttctccctccccaagtgttctcaacctccattctgcagtgttcagagttttagggaaagggtttgggtgccccagcatccaggtgttgtgtggcttagcgcatgtgaagtgaaaaccttctggggttgtttggaagcagctttctggttcttgtgattgtatcctgaggtcccagaaccctattctcccacgaggatcctcagtgaccatggtggccacacgcctggccagcctgctggctcctgggtgagctgaagaaccttgcctgtggcacttttcgagggtgagctggaaccgagagaacatggtccccgtgctgggactcatgcgggtcatttcctgccggcctggtttcgcctggtcgtgtctttatgagcaccatgtaagcctccttgtattgagataattgggcattaaacattaaactgcagctctgggaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 38 - protein, 437 aa, Homo sapiens
MLQGPRALASAAGQTPKVVPAMSPTELWPSGLSSPQLCPATATYYTPLYPQTAPPAAAPGTCLDATPHGPEGQVVRCLPAGRLPAKRKLDLEGIGRPVVPEFPTPKGKCIRVDGLPSPKTPKSPGEKTRYDTSLGLLTKKFIYLLSESEDGVLDLNWAAEVLDVQKRRIYDITNVLEGIQLIRKKAKNNIQWVGRGMFEDPTRPGKQQQLGQELKELMNTEQALDQLIQSCSLSFKHLTEDKANKRLAYVTYQDIRAVGNFKEQTVIAVKAPPQTRLEVPDRTEDNLQIYLKSTQGPIEVYLCPEEVQEPDSPSEEPLPSTSTLCPSPDSAQPSSSTDPSIMEPTASSVPAPAPTPQQAPPPPSLVPLEATDSLLELPHPLLQQTEDQFLSPTLACSSPLISFSPSLDQDDYLWGLEAGEGISDLFDSYDLGDLLIN